(19) United States
(12) Patent Application Publication
(10) Pub. No.: US 2015/0017134 A1
(43) Pub. Date: Jan. 15, 2015

(54) EMT-INDUCING TRANSCRIPTION FACTORS COOPERATE WITH SOX9

(71) Applicant: Whitehead Institute for Biomedical Research, Cambridge, MA (US)

(72) Inventors: Wenjun Guo, New York, NY (US); Robert A. Weinberg, Brookline, MA (US); Zuzana Keckesova, Cambridge, MA (US)

(21) Appl. No.: 14/382,519

(22) PCT Filed: Mar. 1, 2013
(86) PCT No.: PCT/US13/28665

§ 371 Date: Sep. 2, 2014

Related U.S. Application Data
(60) Provisional application No. 61/605,638, filed on Mar. 1, 2012.

(51) Int. Cl. C12N 5/074; A61K 38/17; C12N 15/113; C12Q 1/68; G01N 33/574; A61K 35/28; C12N 5/071

(52) U.S. Cl. CPC C12N 5/0696; A61K 35/28; A61K 38/1709; C12N 5/0631; C12Q 1/6886; G01N 33/57484; C12N 15/1136; C12N 2501/40; C12N 2501/60; C12N 2310/14; C12N 2310/531; C12Q 2600/16; C12Q 2600/158; G01N 2500/10; G01N 2333/46; C12N 2506/45; C12N 2506/095
USPC 424/93.7; 435/377; 514/19.3; 435/325; 435/375; 506/9; 435/29; 435/405; 435/384; 435/6.12; 436/501; 435/7.1; 514/44 A

(57) ABSTRACT

In some aspects, compositions and methods useful for generating stem cells from epithelial cells are disclosed.

### CROSS-REFERENCE TO RELATED APPLICATION

This application claims the benefit of U.S. Provisional Application No. 61/605,638, fled Mar. 1, 2012. The entire teachings of the above application(s) are incorporated herein by reference.

### GOVERNMENT SUPPORT

This invention was made with government support under PO-CA080111 and F32CA144404 awarded by the National Cancer Institute, and R01 CA078461 awarded by the National Institutes of Health. The government has certain rights in the invention.

### BACKGROUND

Adult stem cells (SC) play important roles in the development and maintenance of a number of tissue types. Stem cells also play a role in tumorigenesis. The factors that control adult SC programs are of significant scientific and medical interest.

### SUMMARY

In some aspects, the disclosure provides methods of generating stem cells from epithelial cells. In some aspects, the disclosure provides methods of maintaining stem cells in a stem cell state.

In some aspects, methods of generating stem cells from epithelial cells are disclosed, the methods comprising steps of: (a) providing a population of epithelial cells; and (b) inducing epithelial-mesenchymal transition (EMT) and increasing the amount or activity of at least one EMT-cooperating protein in the population of epithelial cells, thereby generating stem cells in the population. In some embodiments the EMT-cooperating protein is a transcription factor (TF). In some embodiments the EMT-cooperating protein comprises a Sox protein, e.g., Sox9 or Sox10. In some embodiments inducing EMT comprises inducing expression of an EMT-TF. In some embodiments inducing EMT comprises ectopically expressing an EMT-TF. In some embodiments an EMT-TF comprises Slug or Snail, or a functional variant of either. In some embodiments increasing the amount or activity of at least one EMT-cooperating protein in the population of epithelial cells comprises introducing into the cells a nucleic acid that encodes a polypeptide comprising the EMT-cooperating protein (e.g., a polypeptide comprising an EMT-TF) or inducing expression of such a nucleic acid that was previously introduced into ancestor(s) of the cells. In some embodiments inducing EMT comprises introducing into cells a nucleic acid that encodes a polypeptide comprising an EMT-TF or inducing expression of such a nucleic acid that was previously introduced into ancestor(s) of the cells.

In some aspects, isolated epithelial cells comprising an exogenously introduced EMT-inducing agent and an exogenously introduced EMT-cooperating agent are disclosed. In some embodiments the exogenously introduced EMT-inducing agent comprises, encodes, or induces expression of a polypeptide comprising an EMT-TF. In some embodiments the exogenously introduced EMT-cooperating agent comprises, encodes, or induces expression of a polypeptide comprising an EMT-cooperating TF. In some embodiments the exogenously introduced EMT-cooperating agent comprises, encodes, or induces expression of a polypeptide comprising an EMT-cooperating TF comprising a Sox protein or a functional variant thereof. In some embodiments the exogenously introduced EMT-cooperating agent comprises, encodes, or induces expression of a polypeptide comprising an EMT-cooperating TF comprising Sox9 or Sox10 or a functional variant of either.

In some aspects the disclosure provides methods of converting an epithelial cell to a cell having a less differentiated state.

In some aspects the disclosure provides cells generated or maintained according to any of the methods. In some aspects the disclosure provides methods of using such cells. In some aspects the disclosure provides compositions comprising such cells.

In some aspects, the disclosure provides methods of treating a subject. In some embodiments the methods comprise introducing into the subject stem cells, progenitor cells, or differentiated descendants thereof, wherein said cells are generated as described herein. In some embodiments the subject is in need of treatment of cancer, and the methods comprise administering a first agent that inhibits EMT and a second agent that inhibits an EMT-cooperating TF.

In some aspects, the disclosure provides products and compositions useful to perform one or more of the methods.

Certain conventional techniques of cell biology, cell culture, molecular biology, microbiology, recombinant nucleic acid (e.g., DNA) technology, immunology, etc., which are within the skill of the art, may be of use in aspects of the invention. Non-limiting descriptions of certain of these techniques are found in the following publications: Ausubel, F., et al., (eds.), Current Protocols in Molecular Biology, Current Protocols in Immunology, Current Protocols in Protein Science, and Current Protocols in Cell Biology, all John Wiley & Sons, N.Y., editions as of 2008; Sambrook, Russell, and Sambrook, Molecular Cloning: A Laboratory Manual, 3^{rd} ed., Cold Spring Harbor Laboratory Press, Cold Spring Harbor, 2001; Harlow, E. and Lane, D., Antibodies—A Laboratory Manual, Cold Spring Harbor Laboratory Press, Cold Spring Harbor, 1988; Burns, R., Immunochemical Protocols (Methods in Molecular Biology) Humana Press; 3rd ed., 2005, Monoclonal antibodies: practical approach (P. Shepherd and C Dean, eds., Oxford University Press, 2000); Freshney, R. I., “Culture of Animal Cells, A Manual of Basic Technique”, 5th ed., John Wiley & Sons, Hoboken, N.J., 2005; Cancer: Principles and Practice of Oncology (V. T. De Vita et al., eds., J.B. Lippincott Company, 8^{th} ed., 2008). Further information on cancer may be found in The Biology of Cancer, Weinberg, R A, et al., Garland Science, 2006. All patents, patent applications, websites, databases, scientific articles, and other publications mentioned herein are incorporated herein by reference in their entirety. In the event of a conflict or inconsistency with the specification, the specification shall control. The Applicants reserve the right to amend the specification based on any of the incorporated references and/or to correct obvious errors. None of the contents of the incorporated references shall limit the invention.

### BRIEF DESCRIPTION OF THE DRAWING

FIGS. 1A-1D. Slug is the major EMT-TF expressed in MaSCs. (A) The mRNA levels of EMT-TFs expressed in MaSC-enriched basal (stem/basal) or luminal progenitor cells were compared to those of differentiated luminal cells (diff. luminal) by qRT-PCR in triplicate. GAPDH was used as a loading control. This work demonstrated that stem/basal cells relative to differentiated luminal cells express more than 100-fold higher levels per cell of the Slug EMT-TF. Hence, Slug expression is a natural concomitant of the basal/stem-cell state in the mammary gland. (B) Immunofluorescence analyses of the Slug protein expression in mammary gland sections. An anti-keratin 8 antibody was used to label luminal cells. (C) Expression of the Slug-YFP reporter in MaSC-enriched basal (basal/stem), luminal progenitor and differentiated luminal (diff. luminal) cells. This revealed that Slug expression occurs in the basal, abluminal layers of a normal mouse mammary gland. This localization is consistent with the known localization of MaSCs but did not prove the cells expressing the Slug-EMT-TF are stem cells. It did demonstrate however, that expression of this EMT-TF occurs in normal unperturbed tissue that is not associated with neoplasia or inflammation. (D) Gland-reconstituting activities of Slug-YFP⁺ and Slug-YFP⁻ MECs were measured by the limiting dilution analysis. Representative whole-mount images of carmine-stained fat pads (upper panel) and reconstitution efficiencies (lower panel) are shown. In the lower panel, each circle represents one transplanted fat pad; and the dark area of each circle represents the percentage of the fat pad occupied by reconstituted mammary ductal trees. P=1.6×10⁻⁵. Data are represented as mean±SEM. See also FIG. 8. Parts C and D of this Figure demonstrated that a fluorescent marker whose expression is driven by the transcriptional promoter of the normal, native Slug gene is expressed preferentially in cells having MaSC activity, as demonstrated by the elevated ability of such expressing cells to reconstitute an entire mouse mammary gland upon implantation into a cleared mammary stromal fat pad, that is, a stromal microenvironment in which the normally resident mammary epithelial cells (MECs) have been removed and which provides a hospitable tissue environment for the formation of an entire mammary ductal tree by implanted MaSCs. In summary, Slug expression is tightly couple with MaSC activity in mouse MECs.

FIGS. 2A-2E. Ectopic expression of Slug induces MaSC activity. (A) Expression levels of EMT-associated proteins were determined by immunoblot. Primary MECs transduced with tetracycline-inducible Slug lentivirus were treated with the indicated concentration of doxycycline for 5 days. This controlled induction of Slug activity allowed measurement of subsequent cell-biological responses elicited by this induced EMT-TF. (B) Organoid-forming efficiencies of primary MECs transduced with the indicated vectors. This in vitro culture assay represents a surrogate test of MaSC function in vivo, (C) Gland-reconstituting activity was measured by the competitive reconstitution assay. Left panel shows representative whole-mount fluorescence images of mammary fat pads at the indicated time points post-injection. Right panel shows the ratios of GFP-expressing (either vector-control or Slug) to dsRed-expressing cells, as measured by flow cytometry. This experiment provided a test of the notion that transient expression of the Slug EMT-TF in MECs resulted in the acquisition by the subsequently implanted MECs (in the absence of ongoing Slug expression) of gland-reconstituting activity. Hence, any acquired MaSC activity in vivo, including MaSC activity, represented the long-term response of MECs to transient exposure to Slug expression. (D, E) Organoid-forming efficiencies of MaSC-enriched basal cells (D) or luminal progenitor cells (E) transduced with the indicated vectors. Data are represented as mean±SEM. See also FIG. 9.

FIGS. 3A-3E. Cooperation of Sox9 with Slug in the formation of MaSCs. (A) Screening for cofactor(s) of Slug in the induction of organoid-forming cells. Differentiated luminal cells transduced with the indicated vectors were treated with doxycycline for 5 days in monolayer culture and then subjected to organoid culture without further doxycycline treatment. (B) Organoid-forming efficiencies of differentiated luminal cells transduced with the indicated vectors and then treated as in (A). (C) Gland-reconstituting activity of differentiated luminal cells transduced with the indicated vectors. The fat pads were analyzed 7 weeks post-injection by whole-mount analysis (top panel) and flow cytometry (middle panel). The relative MaSC activity was quantified as ratios of GFP- to dsRed-positive cells (lower panel). The data are representative of three independent experiments. (D) Outgrowths generated by GFP-expressing differentiated luminal cells transduced with the indicated vectors. The cells were treated with doxycycline for 8 days in monolayer culture and transplanted into cleared mammary fat pads at limiting dilutions. The fat pads were examined 3 months post-implantation by whole-mount imaging (left and middle panels) or immunofluorescence on tissue sections (right panel). (E) Secondary transplantation generated by Slug/Sox9-exposed differentiated luminal cells. Mice were mated 4 weeks post-transplantation. Mammary fat pads at gestation day ˜18 were then analyzed by whole-mount fluorescent imaging (left) or immunofluorescence on tissue sections (right). Data are represented as mean±SEM. See also FIG. 10.

FIGS. 4A-4C. Induction of MaSCs by Sox9 in basal cells. (A) Organoid-forming efficiencies of basal cells transduced with the indicated vectors. (B) Solid organoid- and acinus-forming efficiencies of basal cells transduced with the indicated cDNA and shRNA expression vectors. Cells were subjected to organoid culture 5 days post-infection. The shLuciferase (shLuc) was used as a control shRNA. Acinus-forming ability is a reflection of the ability to induce the formation of a subset of the cell types in the fully-formed mammary gland. (C) A model showing the mammary epithelial hierarchy and actions of forced expression of Slug and Sox9 in various mammary epithelial lineages. The dashed lines indicate that expression of the indicated factor(s) converts differentiated cells into stem or progenitor cells. Data are represented as mean±SEM.

FIGS. 5A-5C. Slug and Sox9 are required for maintaining endogenous MaSCs. (A) Confocal immunofluorescence analyses of mammary gland sections stained with rabbit anti-Slug and goat anti-Sox9 antibodies. Arrows point to Slug and Sox9 double-positive nuclei. (B) Organoid-forming efficiencies of primary MECs transduced with the indicated shRNA vectors. Cells were subjected to organoid culture 4 days post-infection. (C) Gland-reconstituting activity of primary MECs transduced with the indicated shRNA vectors. The shRNA-vector-transduced and GFP-expressing primary MECs (1×10⁵) were mixed with equal number of dsRed-expressing MECs and then transplanted into cleared mammary fat pads. The ratios of GFP-versus dsRed-positive cells were normalized against that of the shLuc control to obtain relative reconstitution efficiency. Data are represented as mean±SEM. See also FIG. 11.

FIGS. 6A-6E. Slug and Sox9 activate distinct auto-regulatory gene expression programs. (A) Phase-contrast and immunofluorescence images of differentiated luminal cells expressing the indicated vectors for 4 (phase-contrast) or 5 days (immunofluorescence). (B and C) The mRNA levels of basal cell TFs (B) and luminal progenitor genes (C) in differentiated luminal cells expressing the indicated vectors for 5 days, as measured by qRT-PCR. GAPDH was used as a loading control. (D) mRNA levels of various signature genes in cells after a 6-day doxycycline treatment (Slug/Sox9 on dox) or a 6-day doxycycline treatment plus a 6-day doxycycline withdrawal (Slug/Sox9 dox withdrawal). The mRNA levels were normalized to those of control-vector-transduced cells after the 6-day doxycycline treatment. Primers amplifying protein-coding sequences were used to detect total mRNA levels of Slug and Sox9 (total); and primers amplifying the 5′UTRs were used to detect endogenously-expressed Slug and Sox9 mRNAs (endo). (E) Organoid-forming efficiencies of differentiated luminal cells transduced with the indicated vectors after a 5-day doxycycline treatment (on dox) or a 5-day treatment plus a 6-day withdrawal (dox withdrawal) in monolayer culture. Data are represented as mean±SEM. See also FIG. 12.

FIGS. 7A-7D. Slug and Sox9 act as regulators of breast CSCs. (A) Tumor weight and incidence of MDA-MB-231 cells expressing the indicated shRNAs. Cells were injected subcutaneously at the indicated numbers. Tumor weight and incidence were determined three months post-injection. Each data point represents one tumor. The mean and SEM of each group was represented by horizontal and vertical bars. The table shows tumor incidence. (B) Lung metastases formed by MDA-MB-231 cells expressing the indicated shRNAs vectors upon tail vein injection. (C) Lung metastases formed by tdTomato-labeled MCF7ras cells that were transduced with the indicated vectors and injected orthotopically into mammary fat pads. Whole-mount fluorescence lung images and histology of lung sections are show. n=4 for each group. The data are representative of two independent experiments. (D) Cumulative survival rate of human breast cancer patients with primary tumors expressing high levels of both Slug and Sox9 (Slug/Sox9-high) or tumors expressing only one factor or neither factors at high levels (Non-Slug/Sox9-high). Data are presented as mean±SEM. See also FIG. 13.

FIGS. 8A-8E. Slug is the major EMT-TF expressed in MaSCs. (A) A schematic diagram of the mammary epithelial hierarchy (top left) and FACS profiles of freshly isolated MECs stained simultaneously for CD29, CD49f and CD61. The Lin⁻ EpCAM⁺ single MECs were plotted for the CD49f/CD61 expression (top right) or the CD29/CD61 expression (bottom). These two types of analyses yielded similar three-population profiles. When individual populations gated based on the CD49f/CD61 expression were analyzed for the CD29/CD61 expression, the populations identified by CD49f/CD61 superimposed with the corresponding populations identified by CD29/CD61. (B) Cleared mammary fat pad reconstitution ability of various MEC subpopulations injected at limiting dilutions. The upper panel shows representative images of fat pads that had been injected with 1×10⁴ cells. The lower panel shows reconstitution efficiency of various cell populations injected at the indicated number. (C) Acinus-forming efficiencies of CD49f^{low}CD61⁺ luminal progenitor cells and CD49f^{low}CD61⁻ differentiated luminal cells. Sorted MECs were cultured in Matrigel as described in (Asselin-Labat et al., 2007). Similar to cells sorted based on CD29 and CD61 (Asselin-Labat et al. 2007), CD49f^{low}CD61⁺ luminal progenitor cells efficiently formed acinar structures in Matrigel culture that are indicative of progenitor activities, whereas CD49f^{low}CD61⁻ differentiate luminal cells could only do so with far lower frequencies (20-fold less). Data are presented as mean±SEM. (D) The relative mRNA levels of various EMT markers in mouse MEC subpopulations, as measured by qRT-PCR. GAPDH was used as a loading control. Data are presented as mean±SEM. (E) The expression levels of EMT-TFs in human MEC subpopulations were taken from a public gene expression microarray dataset (GSE16997 from NCBI GEO) (Lim et al., 2010). The expression level of each gene in MaSC-enriched basal cells (stem/basal) or luminal progenitor cells was compared to that of differentiated luminal cells (diff. luminal). The mean values of 3 independent human samples are shown. Differential expression relative to the diff. luminal population was assayed with a moderated t-test as implemented by limma (Smyth, 2004).

FIGS. 9A-9E. Ectopic expression of Slug induces MaSC activity. (A) Representative images of 3-dimensional structures formed by MaSC-enriched basal cells (stem/basal) and luminal progenitor cells in Matrigel organoid culture. Images on the right are magnifications of the selected areas on the left. (B) Gland-reconstituting ability of solid organoids generated from single MaSC-enriched basal cells, Nine primary organoids generated from single cells were dissociated separately, and the resulting cells were re-seeded to generate secondary organoid cultures. The gland-reconstituting ability of each secondary organoid culture was examined by injecting 25% of the culture into a cleared mammary fat pad. Six out of nine cultures generated fully-reconstituted mammary ductal trees. Some of the recipients were impregnated to induce alveologenesis. (C) Gland-reconstituting ability of acini that were generated from single luminal progenitor cells through the same procedure as in (B). Representative images of cleared fat pads transplanted with acini were shown. In most of cases, acini did not form any reconstitution. Occasionally (⅕), acini formed small rudimentary ductal structures, which had few or no branches and were most likely generated by progenitor cells. (D) Phase-contrast and immunofluorescence images of primary MECs that were transduced with the indicated vectors and treated with doxycycline for 5 days. (E) A schematic diagram of the competitive reconstitution assay. GFP-expressing experimental cells whose MaSC activity needed to be determined were mixed with equal number of competing dsRed-expressing primary MECs and transplanted into cleared mammary fat pads. The reconstitution efficiency of GFP-expressing experimental cells was determined by the ratio of GFP- to dsRed-expressing cells as measured by flow cytometry. In FIG. 2C, the GFP-expressing cells engrafted less efficiently than the dsRed-expressing cells (see ratios at days 1 and 7), which was likely due to harmful effects of viral infection on the GFP-expressing cells.

FIGS. 10A-10F. Cooperation of Sox9 with Slug in the formation of MaSCs. (A) The mRNA levels of Slug and Sox9 expressed in cells as shown in FIG. 3B. (B) Acinus-forming efficiencies of differentiated luminal cells transduced with the indicated vectors and treated as shown in FIG. 3B. (C) Organoid-forming efficiencies of differentiated luminal cells transduced with the indicated doxycycline-inducible vectors. The cells were treated with doxycycline for 6 days in monolayer culture and then subjected to organoid culture in the absence of doxycycline. (D) Phase-contrast images of differentiated luminal cells treated as in (C) in monolayer culture. (E) Cleared mammary fat pad reconstitution efficiencies of cells injected as in FIG. 3D. (F) Immunofluorescence analyses of sections of the outgrowths as shown in FIG. 3D. Of note, in outgrowths formed by Slug/Sox9-exposed cells, the expression of Slug and Sox9 was silenced in most cells, reverting to the expression patterns of Slug and Sox9 observed in normal mammary glands (FIG. S3F and FIG. 5A). This indicates that the expression of exogenous Slug and Sox9 was successfully silenced. In addition, it suggests that contextual signals in mammary glands could control the expression of endogenous Slug and Sox9 that had been previously induced by the exogenous Slug and Sox9 (see FIG. 6D), therefore encouraging and permitting proper differentiation. Consistent with this, the outgrowths exhibited normal epithelial architecture, as revealed by the intact adherens junctions formed by E-cadherin and the tight junctions formed by ZO-1 at the luminal layer (FIG. S3F). This demonstrated that the previously induced EMT was reversed during the differentiation of induced MaSCs to luminal cells. Data are presented as mean±SEM.

FIGS. 11A-11D. Slug and Sox9 are required for maintaining endogenous MaSCs. (A) Single-molecule fluorescence in situ hybridization (FISH) for detecting Slug and Sox9 transcripts in mammary gland sections. Fluorescent dots represent single Sox9 (red) or Slug (green) transcripts detected by FISH probes. Dashed lines mark single cells based on DAPI fluorescence. The image is a projection of 5 confocal Z stacks spaced 0.3 micron apart and filtered with a Laplacian of Gaussian filter with a standard deviation of 1.5 pixels to enhance contrast. The arrow points to a Slug/Sox9 double-positive cell. About 6% of all MECs and 15% of basal cells expressed high levels (>mean transcript concentration) of both Slug and Sox9. (B) Knockdown efficiencies of Slug and Sox9 in primary MECs as measured by immunoblot. (C and D) Primary MECs expressing the indicated shRNA vectors were seeded at 2000 cells per well in organoid culture (B) or monolayer culture (C). The total number of cells in each well was quantified 13 days post-seeding. Data are presented as mean±SEM.

FIGS. 12A-12G. Slug and Sox9 activate distinct auto-regulatory gene expression programs. (A) Immunoblot analyses of EMT markers in differentiated luminal cells transduced with the indicated vectors. (B) Relative expression levels of genes associated with basal cells or luminal progenitor cells were determined by qRT-PCR. The expression levels in MaSC-enriched basal cells (stem/basal) or luminal progenitors were compared to those of differentiated luminal cells (diff. luminal). GAPDH was used as a loading control. The same Twist2 data were also shown in FIG. 1A. (C) Immunoblot analyses of Sox9 and Slug expression in differentiated luminal cells treated as in FIG. 6D. (D) Organoid-forming efficiencies. Differentiated luminal cells were first transduced with the indicated doxycycline-inducible cDNA expression vectors and treated with doxycycline for 6 days. The cells were then either subjected to organoid culture (on dox) or further transduced with the indicated shRNA vectors and cultured without doxycycline in monolayer for 6 days before subjected to organoid culture (dox withdrawal). (E and F) Organoid-forming efficiencies (upper panels) and shRNA-knockdown efficiencies (lower panels). Differentiated luminal cells were transduced concomitantly with the indicated shRNA vectors and doxycycline-inducible cDNA expression vectors. The cells were treated with doxycycline for 7 days in monolayer culture and then subjected to organoid culture. (G) Organoid-forming efficiencies. Differentiated luminal cells were transduced with the indicated doxycycline-inducible cDNA expression vectors. The cells were then treated with doxycycline for 6 days in monolayer culture and then subjected to organoid culture. Data are presented as mean±SEM.

FIGS. 13A-13G). Slug and Sox9 act as regulators of breast CSCs. (A) Knockdown efficiency of Slug and Sox9 in MDA-MB-231 cells as determined by immunoblot. Normal human MECs immortalized by telomerase (HME) were used as a control for the Sox9 protein expressed in normal MECs. MDA-MB-231 cells express a Sox9 isoform that is ˜10 kDa smaller than the corresponding isoform in HME cells. (B) Tumor-initiating ability of MDA-MB-231 cells as shown in FIG. 7A. (C) Growth curves of MDA-MB-231 cells infected with the indicated shRNA vectors in monolayer culture. Cells were seeded at 1×10⁴ cells per well in 6-well plates. The number of cells in each well was quantified at the indicated time points post-seeding. (D) Expression of Slug and Sox9 proteins in MCF7ras cells transduced with the indicated vectors. The cells were treated with doxycycline or left untreated for 5 days in monolayer culture. The β-actin protein was used as a loading control. (E) Immunofluorescence analyses of EMT markers. MCF7ras cells treated with doxycycline for 2 weeks in vivo were FACS sorted based on the tdTomato expression. The cells were cultured in monolayer in the presence of doxycycline for 2 days and then fixed for immunofluorescence analyses. (F) The weight of primary tumors generated by MCF7ras cells as shown in FIG. 7C. (G) Representative images of human breast cancer samples expressing various levels of Slug and Sox9 as indicated. Data are presented as mean±SEM.

### DETAILED DESCRIPTION

### I. Glossary

Certain terms used in the present disclosure and related description are collected here for purposes of convenience.

“Agent” as used herein encompasses proteins, small molecules, nucleic acids, lipids, supramolecular complexes, entities such as viruses or portions thereof, and other biological or chemical entities that can be contacted with cells ex vivo or administered to a subject. An “agent” may comprise multiple different agents of distinct structure or sequence. The term “agent” may be used interchangeably with the term “compound” herein. In general, an agent disclosed herein can be prepared or obtained using any of a variety of methods. Methods suitable for preparation of particular agents or types of agents are known to those of ordinary skill in the art. For example, in various embodiments an agent is isolated from an organism that naturally contains or produces it (e.g., plants, animals, fungi, bacteria). In some embodiments an agent is at least partly synthesized, e.g., using chemical or biological methods. In some embodiments recombinant nucleic acid technology is used to produce an agent, e.g., a gene expression product such as an RNA or protein. Methods for generating genetically modified cells or organisms, e.g., cells (prokaryotic or eukaryotic) or organisms (e.g., animals, plants) that can serve as sources of the agent are known to those of ordinary skill in the art. Exemplary methods are described in various references cited herein. In some embodiments a protein or nucleic acid has or comprises a naturally occurring sequence. In some embodiments a protein or nucleic acid comprises or has a sequence that is at least in part invented or generated by man and/or not known to be found in nature. In some embodiments an agent or composition herein comprises a naturally occurring polypeptide. For purposes herein, a polypeptide is said to be “naturally occurring” if it has the amino acid sequence of a polypeptide found in nature. For example, a recombinantly produced polypeptide identical in sequence to a polypeptide found in nature is said to be a “naturally occurring” polypeptide. In some embodiments, a variant of a naturally occurring polypeptide is used. In some embodiments an agent disclosed herein or used in a method or composition herein (i.e., any such agent) is an isolated or purified agent.

“Antibody” encompasses immunoglobulins and derivatives thereof containing an immunoglobulin domain capable of binding to an antigen. An antibody can originate from a mammalian or avian species, e.g., human, rodent (e.g., mouse, rabbit), goat, camelid, chicken, etc., or can be generated ex vivo using a technique such as phage display. Antibodies are of use in certain embodiments. Antibodies include members of the various immunoglobulin classes, e.g., IgG, IgM, IgA, IgD, IgE, or subclasses thereof such as IgG1, IgG2, etc., and, in various embodiments, encompasses antibody fragments or molecules such as an Fab′, F(ab′)2, scFv (single-chain variable) that retains an antigen binding site and encompasses recombinant molecules comprising one or more variable domains (VH or VL). An antibody can be monovalent, bivalent or multivalent in various embodiments. An antibody may be a chimeric or “humanized” antibody. In some embodiments an antibody is a fully humanized antibody. An antibody may be polyclonal or monoclonal, though monoclonal antibodies may be preferred in certain embodiments.

“Cellular marker” or simply “marker” refers to a molecule (e.g., a protein, RNA, DNA, lipid, carbohydrate) or portion thereof, the level of which in or on a cell (e.g., at least partly exposed at the cell surface) that can be detected or measured by available methods and that characterizes, indicates, or identifies one or more cell type(s), cell lineage(s), or tissue type(s) or characterizes, indicates, or identifies a particular state (e.g., a diseased or physiological state such as cancerous or normal, a differentiation state, a stem cell state). A level may be reported in a variety of different ways, e.g., high/low; +/−; numerically, etc. The presence, absence, or level of certain cellular marker(s) may indicate a particular physiological or differentiated or diseased state of a patient, organ, tissue, or cell. It will be understood that multiple cellular markers may be assessed in concert in order to, e.g., identify or isolate a cell type of interest, diagnose a disease, etc. In some embodiments between 2 and 10 cellular markers may be assessed. A cellular marker present on or at the surface of cells may be referred to as a “cell surface marker”. In some embodiments, a cell surface marker is a receptor. For example, a targeting moiety may bind to an extracellular domain of a receptor. A cellular marker may be cell type specific. A cell type specific marker is generally expressed or present at a higher level in or on (at the surface of) a particular cell type or cell types than in or on many or most other cell types (e.g., other cell types in the body or in an artificial environment). In some cases a cell type specific marker is present at detectable levels only in or on a particular cell type of interest. However, useful cell type specific markers may not be and often are not absolutely specific for the cell type of interest. A cellular marker, e.g., a cell type specific marker, may be present at measurable levels that are at least 2-fold or at least 3-fold greater in or on the surface of a particular cell type than in a reference population of cells which may consist, for example, of a mixture containing cells from multiple (e.g., 5-10; 10-20, or more) of different tissues or organs in approximately equal amounts. In some embodiments a cellular marker, e.g., a cell type specific marker, may be present at measurable levels that are at least 4-5 fold, between 5-10 fold, or more than 10-fold greater than its average expression in a reference population. In some embodiments a cellular marker, e.g., a cell surface marker, is selectively expressed by tumor cells, e.g., is overexpressed by tumor cells as compared with expression by normal cells, e.g., normal cells derived from the same organ and/or cell type. Such a cellular marker may be referred to as a “tumor cellular marker”. A tumor marker present on or at the surface of a tumor cell may be referred to as a “tumor cell surface marker”. In general, the level of a cellular marker may be determined using standard techniques such as Northern blotting, in situ hybridization, RT-PCR, sequencing, immunological methods such as immunoblotting, immunohistochemistry, fluorescence detection following staining with fluorescently labeled antibodies (e.g., flow cytometry, fluorescence microscopy), similar methods using non-antibody ligands that specifically bind to the marker, oligonucleotide or cDNA microarray or membrane array, protein microarray analysis, mass spectrometry. A cell surface marker, e.g., a cell type specific cell surface marker or a tumor cell surface marker, may be used to detect or isolate cells or as a target in order to deliver an agent to cells. For example, the agent may be linked to a moiety that binds to a cell surface marker. Suitable binding moieties include, e.g., antibodies or ligands, e.g., small molecules, aptamer, polypeptides.

“Cooperate”, “cooperation” and like terms as used herein refer to a situation in which two or more pathways, processes, or agents each contribute to an effect, outcome, result, phenotype, change in state, maintenance of a state, etc. The terms “cooperate with” and “collaborate with” are used interchangeably herein. In some embodiments a first process or pathway is said to “cooperate with” a second process or pathway if (i) the two processes or pathways in combination produce an effect greater than or qualitatively different from that which results from either pathway or process in the absence or substantial absence of the other; (ii) one process or pathway is necessary in order for the other to occur; (iii) both processes or pathways must remain active in order to maintain a particular state or effect of interest; and/or (iv) both processes or pathways must be expressed concomitantly or in sequence for a limited period of time, after which the consequences of their expression may be measurable at great delay (e.g., days or weeks) after the expression of both processes is terminated. The term “in combination” or “concomitantly” as used in regard to processes or pathways or modulation of processes or pathways refers to occurring during at least partly overlapping in time periods or sufficiently close together in time such that an effect of a first process or pathway or an effect of modulating a process or pathway remains at least partly detectable at the time the second process or pathway is modulated, e.g., induced or activated. In some embodiments a first agent is said to “cooperate with” a second agent if (i) the two agents when present or used in combination produce an effect greater than or qualitatively different from that which results from either agent individually at the same concentration or amount (assuming otherwise similar conditions) or (ii) both agents (or agents providing comparable or approximately equivalent activities) must be present in order to maintain a particular state or effect. An agent that can induce, activate, or bring about a first process or pathway that cooperates with a second process or pathway is said to cooperate with the second process or pathway. It will be understood that cooperation is mutual, e.g., if a first process, pathway, agent cooperates with a second process, pathway, or agent, then the second process, pathway, or agent cooperates with the first process, pathway, or agent. It will also be understood that any process, pathway, or agent may be considered a first or second process, pathway, or agent, respectively. In some embodiments agents are considered to be present or used “in combination” if they are present, used, or active within at least partly overlapping time periods or sufficiently close together in time such that an effect of a first agent remains at least partly detectable at the time the second agent is first present, used, or active. In some embodiments two or more agents in combination produce an effect that is greater than the sum of their individual effects. Such an effect may be referred to as “synergy”. Similarly, in some embodiments two or more processes or pathways in combination (or a pathway or process in combination with an agent or vice versa) produce an effect that is greater than the sum of their individual effects.

“Differentiation potential” as used herein refers to the capacity of a stem cell or progenitor cell to give rise to cells that are more differentiated than the stem cell or progenitor cell itself. A first cell is said to have “expanded differentiation potential” or “increased differentiation potential” as compared to a second cell if the first cell is capable of giving rise to a greater number of distinct differentiated cell types than the second cell. For example, a cell that is capable of giving rise to differentiated luminal cells and to differentiated myoepithelial cells is considered to have greater differentiation potential than a cell that is capable of giving rise only to differentiated luminal cells.

An “EMT-TF” is a transcription factor (TF) that is capable, through its pleiotropic actions, of activating or inducing expression of the cell-biological program termed the epithelial-mesenchymal transition (EMT).

An “EMT-cooperating TF refers” to a TF that is able to cooperate with an EMT-TF to facilitate entrance into the stem cell (SC) state.

“Expanding the differentiation potential of a cell” refers to converting a cell into a cell that has expanded differentiation potential. It will be understood that the process of converting may in some embodiments occur over a time period encompassing one or more cell division cycles, and in such cases in some embodiments only some but not all of the resulting cells may have an expanded differentiation potential. It will also be understood that, in general, when the differentiation potential of two cells is compared, it is assumed that the comparison is performed on the cells in their existing state, without subjecting the cells to manipulation that would alter their differentiation potential.

An “effective amount” or “effective dose” of an agent (or composition containing such agent) generally refers to the amount sufficient to achieve a desired biological and/or pharmacological effect, e.g., when contacted with a cell in vitro or administered to a subject according to a selected administration form, route, and/or schedule. As will be appreciated by those of ordinary skill in the art, the absolute amount of a particular agent or composition that is effective may vary depending on such factors as the desired biological or pharmacological endpoint, the agent to be delivered, the target tissue, etc. Those of ordinary skill in the art will further understand that an “effective amount” may be contacted with cells or administered in a single dose, or through use of multiple doses, in various embodiments. It will be understood that agents, compounds, and compositions herein may be employed in an amount effective to achieve a desired biological and/or therapeutic effect.

“Endogenous” generally refers to an agent, e.g., a molecule, that is native to cells or organisms that contain and/or produce it and was not introduced directly or indirectly by the hand of man or by another external event into the cell or organism or an ancestor of the cell or organism. For example, a nucleic acid or protein that is naturally encoded by the genome of a cell or organism that produces it or in which it is found (i.e., not as a result of genetic engineering or other manipulation affecting the genome) is considered endogenous to the cell or organism. One of ordinary skill in the art will appreciate that certain naturally occurring events such as infection by certain viruses or naturally occurring introduction of genetic elements may have occurred so many generations or years ago (e.g., at least thousands of years ago) that the inherited genetic material may be considered endogenous to the cell or species that contains it. For purposes hereof, the term “endogenous” is often used to refer to molecules, e.g., RNA or proteins, that are encoded in the naturally existing genome of cells or organisms of interest without introduction of a nucleic acid encoding such molecules into such cells or organisms (or into an ancestor of the cell or organism) by the hand of man. “Endogenous expression” refers to expression arising from an endogenous gene. “Exogenous” generally refers to an agent, e.g., a molecule, that is introduced directly or indirectly by the hand of man or another external event into a cell or organism that contains or produces it (or into an ancestor of the cell or organism). For example, a nucleic acid that has been introduced into a cell or organism (or into an ancestor of the cell or organism) is considered an exogenous nucleic acid as are copies of such nucleic acid, e.g., a copy that has integrated into the genome, been transcribed, reverse transcribed, copied, and/or inherited. An expression product resulting from expression (e.g., transcription, translation, or transcription and translation of the resulting transcript) of an exogenous nucleic acid is considered exogenous. As will be understood by those of ordinary skill in the art, an exogenous molecule may be identical to an endogenous molecule. For example, an exogenous nucleic acid may encode a protein identical in sequence to a protein encoded by the unmodified genome of a cell or organism. “Ectopic expression” as used herein refers to expression arising from an exogenous nucleic acid. In some embodiments expression is induced by activating expression of an endogenous gene. In some embodiments such activating may be performed by genetically modifying a cell. In some embodiments such activating is performed without genetically modifying a cell.

The term “expression” encompasses the processes by which nucleic acids (e.g., DNA) are transcribed to produce RNA, and (where applicable) RNA transcripts are processed and/or translated into polypeptides, e.g., in a cell.

The term “gene product” (also referred to herein as “gene expression product” or “expression product”) encompasses products resulting from expression of a gene, such as RNA transcribed from a gene and polypeptides arising from translation of such RNA. It will be appreciated that certain gene products may undergo processing or modification, e.g., in a cell. For example, RNA transcripts may be spliced, polyadenylated, etc., prior to mRNA translation, and/or polypeptides may undergo co-translational or post-translational processing such as removal of secretion signal sequences, removal of organelle targeting sequences, or modifications such as phosphorylation, fatty acylation, etc. The term “gene product” encompasses such processed or modified forms. Genomic, mRNA, polypeptide sequences from a variety of species, including human, are known in the art and are available in publicly accessible databases such as those available at the National Center for Biotechnology Information (www.ncbi.nih.gov) or Universal Protein Resource (www.uniprot.org). Exemplary databases include, e.g., GenBank, RefSeq, Gene, UniProtKB/SwissProt, UniProtKB/Trembl, and the like. In general, sequences, e.g., mRNA and polypeptide sequences, in the NCBI Reference Sequence database may be used as gene product sequences for a gene of interest. It will be appreciated that multiple alleles of a gene may exist among individuals of the same species. For example, differences in one or more nucleotides (e.g., up to about 1%, 2%, 3-5% of the nucleotides) of the nucleic acids encoding a particular protein may exist among individuals of a given species. Due to the degeneracy of the genetic code, such variations often do not alter the encoded amino acid sequence, although DNA polymorphisms that lead to changes in the sequence of the encoded proteins can exist. Examples of polymorphic variants can be found in, e.g., the Single Nucleotide Polymorphism Database (dbSNP), available at the NCBI website at www.ncbi.nlm.nih.gov/projects/SNP/. (Sherry S T, et al. (2001). “dbSNP: the NCBI database of genetic variation”. Nucleic Acids Res. 29 (1): 308-311; Kitts A, and Sherry S, (2009). The single nucleotide polymorphism database (dbSNP) of nucleotide sequence variation in The NCBI Handbook [Internet]. McEntyre J, Ostell J, editors. Bethesda (MD): National Center for Biotechnology Information (US); 2002 (www.ncbi.nlm.nih.gov/bookshelf/br.fcgi?book=handbook&part=ch5). Multiple isoforms of certain proteins may exist, e.g., as a result of alternative RNA splicing or editing. In general, where aspects of this disclosure pertain to a gene or gene product, embodiments pertaining to allelic variants or isoforms are encompassed unless indicated otherwise. Certain embodiments may be directed to particular sequence(s), e.g., particular allele(s) or isoform(s).

In some embodiments, if multiple different isoforms of a particular protein are known, an isoform having the highest activity of interest or the most abundant isoform is used in a composition, product, or method described herein. For example, in the case of an EMT-TF, in some embodiments an isoform having the greatest ability to induce EMT is used. In some embodiments the most abundant isoform in a cell type of interest is used. In the case of an EMT-cooperating TF, in some embodiments an isoform having the greatest ability to cooperate with EMT in the generation of stem cells is used. In some embodiments an isoform that is naturally present in stem cells that are precursors to a particular differentiated epithelial cell type is used. For example, in some embodiments, if mammary stem cells are being generated from differentiated mammary epithelial cells, an isoform that is naturally present in mammary stem cells is used. In some embodiments, if multiple isoforms are naturally present, an isoform having the highest expression level or highest activity may be used.

“Identity” or “percent identity” is a measure of the extent to which the sequence of two or more nucleic acids or polypeptides is the same. The percent identity between a sequence of interest A and a second sequence B may be computed by aligning the sequences, allowing the introduction of gaps to maximize identity, determining the number of residues (nucleotides or amino acids) that are opposite an identical residue, dividing by the minimum of TG_{A} and TG_{B} (here TG_{A} and TG_{B} are the sum of the number of residues and internal gap positions in sequences A and B in the alignment), and multiplying by 100. When computing the number of identical residues needed to achieve a particular percent identity, fractions are to be rounded to the nearest whole number. Sequences can be aligned with the use of a variety of computer programs known in the art. For example, computer programs such as BLAST2, BLASTN, BLASTP, Gapped BLAST, etc., may be used to generate alignments and/or to obtain a percent identity. The algorithm of Karlin and Altschul (Karlin and Altschul, Proc. Natl. Acad. Sci. USA 87:22264-2268, 1990) modified as in Karlin and Altschul, Proc. Natl. Acad Sci. USA 90:5873-5877, 1993 is incorporated into the NBLAST and XBLAST programs of Altschul et al. (Altschul, et al., J. Mol. Biol. 215:403-410, 1990). In some embodiments, to obtain gapped alignments for comparison purposes, Gapped BLAST is utilized as described in Altschul et al. (Altschul, et al. Nucleic Acids Res. 25: 3389-3402, 1997). When utilizing BLAST and Gapped BLAST programs, the default parameters of the respective programs may be used. See the Web site having URL www.ncbi.nlm.nih.gov and/or McGinnis, S. and Madden, T L, W20-W25 Nucleic Acids Research, 2004, Vol. 32, Web server issue. Other suitable programs include CLUSTALW (Thompson J D, Higgins D G, Gibson T J, Nuc Ac Res, 22:4673-4680, 1994) and GAP (GCG Version 9.1; which implements the Needleman & Wunsch, 1970 algorithm (Needleman S B, Wunsch C D, J Mol Biol, 48:443-453, 1970.) Percent identity may be evaluated over a window of evaluation. In some embodiments a window of evaluation may have a length of at least 10%, 15%, 20%, 25%, 30%, 35%, 40%, 45%, 50%, 55%, 60%, 65%, 70%, 75%, 80%, 85%, 90%, 95%, 96%, 97%, 98%, 99%, or more, e.g., 100%, of the length of the shortest of the sequences being compared. In some embodiments a window of evaluation is at least 100; 200; 300; 400; 500; 600; 700; 800; 900; 1,000; 1,200; 1,500; 2,000; 2,500; 3,000; 3,500; 4,000; 4,500; or 5,000 amino acids. In some embodiments no more than 20%, 10%, 5%, or 1% of positions in either sequence or in both sequences over a window of evaluation are occupied by a gap. In some embodiments no more than 20%, 10%, 5%, or 1% of positions in either sequence or in both sequences are occupied by a gap.

“Inhibit” may be used interchangeably with terms such as “suppress”, “decrease”, “reduce” and like terms, as appropriate in the context. It will be understood that the extent of inhibition may vary. For example, inhibition may refer to a reduction of the relevant level by at least about 5%, 10%, 15%, 20%, 25%, 30%, 35%, 40%, 45%, 50%, 55%, 60%, 65%, 70%, 75%, 80%, 85%, 90%, 95%, 96%, 97%, 98%, or 99%. In some embodiments inhibition refers to a decrease of 100%, e.g., to background levels or undetectable levels. The term “inhibitor” encompasses agents that inhibit (decrease, reduce) the expression or activity of a target molecule. The term “inhibitor” encompasses agents that inhibit expression and/or inhibit one or more activities of a molecule or complex of interest (the “target”). For example, in various embodiments an agent is an “inhibitor” of a target if one or more activities of the target is reduced in the presence of the compound, or as a consequence of its use, as compared with in the absence of the compound, and/or if the level or amount of the target is reduced in the presence of the compound, or as a consequence of its use, as compared with in the absence of the compound. In certain embodiments, an inhibitor acts directly on a target in that it physically interacts with the target. In some embodiments, an inhibitor acts indirectly, e.g., by inhibiting a second molecule that is needed for synthesis or activity of the target. In some embodiments, an inhibitor is an antagonist. Methods of inhibiting encompass methods that result in a decreased amount of a target and methods that interfere with one or more functions (activities) of a target. In some embodiments, a target is inhibited by inhibiting or interfering with its expression or post-translational processing, so that a decreased amount of functional target is produced, resulting in a decreased overall activity of the target in a cell or system. A variety of methods useful for inhibiting or interfering with expression can be used in various embodiments. In general, such methods result in decreased synthesis of a mRNA and/or polypeptide and as a result, a reduction in the total level of activity present. Other means of inhibition include interfering with proper localization, secretion, or co- or post-translational processing, or promoting increased degradation. Methods of inhibiting activity can include binding to a target or to a receptor or co-receptor for the target and thereby blocking the target from interacting with its receptor(s) or with other molecule(s) needed for activity of the target. In some embodiments an inhibitor binds to an active site or catalytic residue or substrate binding site of an enzyme or blocks dimerization or other protein-protein interactions, etc. For example, in some embodiments a TF that acts as a dimer is inhibited using an agent that blocks dimerization. In some embodiments, an inhibitor comprises an RNAi agent, e.g., an siRNA or shRNA, or an antisense oligonucleotide, that inhibits expression of a target. In some embodiments, an inhibitor comprises an antibody or aptamer or small molecule that binds to and inhibits a target. In some embodiments an inhibitor comprises an agent that acts in a dominant negative fashion to inhibit a target. A dominant negative agent may comprise a fragment of a target molecule that lacks one or more domains necessary for function. For example, in some embodiments a dominant negative form of a TF comprises a DNA binding domain and/or dimerization domain but lacks an activation domain.

“Isolated”, as used herein, means 1) separated from at least some of the components with which it is usually associated in nature; 2) prepared or purified by a process that involves the hand of man; and/or 3) not occurring in nature, e.g., artificial, synthetic, or (iv) present in an artificial environment. Unless otherwise indicated or evident from the context, any agent, product or composition disclosed herein can in certain embodiments be isolated or composed at least in part of isolated component(s).

“Ligand” refers to an agent (e.g., a molecule or complex) that binds to another entity (e.g., a molecule or complex), such as a cellular receptor. The term “agonist” refers to an agent (e.g., a molecule or a complex) that binds to a cellular receptor or receptor complex and triggers a response by the cell, e.g., stimulates a signaling pathway. An “antagonist” is an agent that blocks or otherwise antagonizes the activity of an agonist. For example, an antagonist may bind to the same receptor as an agonist (or to a co-receptor) but fail to elicit the response typically caused by the agonist (and such binding interferes with binding of the agonist), or the antagonist may bind to an agonist and prevent the agonist from binding to the receptor. In some embodiments a ligand as used herein is an agonist.

“Modulate”, “modulating”, “modulation” and like terms, as used herein, encompass inhibiting (reducing, suppressing) or enhancing (activating, promoting, increasing) expression or activity of, e.g., a molecule, complex, pathway, or process.

“Nucleic acid” is used interchangeably with “polynucleotide” and encompasses polymers of nucleotides. “Oligonucleotide” refers to a relatively short nucleic acid, e.g., typically between about 4 and about 100 nucleotides (nt) long, e.g., between 8-60 nt or between 10-40 nt long. Nucleotides include, e.g., ribonucleotides or deoxyribonucleotides. In some embodiments a nucleic acid comprises or consists of DNA or RNA. In some embodiments a nucleic acid comprises or includes only standard nucleobases (often referred to as “bases”). The standard bases are cytosine, guanine, adenine (which are found in DNA and RNA), thymine (which is found in DNA) and uracil (which is found in RNA), abbreviated as C, G, A, T, and U, respectively. In some embodiments a nucleic acid may comprise one or more non-standard nucleobases, which may be naturally occurring or non-naturally occurring (i.e., artificial; not found in nature) in various embodiments. In some embodiments a nucleic acid may comprise chemically or biologically modified bases (e.g., alkylated (e.g., methylated) bases), modified sugars (e.g., 2′-O-alkyribose (e.g., 2′-O methylribose), 2′-fluororibose, arabinose, or hexose), modified phosphate groups (e.g., phosphorothioates or 5′-N-phosphoramidite linkages). In some embodiments a nucleic acid comprises subunits (residues), e.g., nucleotides, that are linked by phosphodiester bonds. In some embodiments, at least some subunits of a nucleic acid are linked by a non-phosphodiester bond or other backbone structure. In some embodiments, a nucleic acid comprises a locked nucleic acid, morpholino, or peptide nucleic acid. A nucleic acid may be linear or circular in various embodiments. A nucleic acid may be single-stranded, double-stranded, or partially double-stranded in various embodiments. An at least partially double-stranded nucleic acid may be blunt-ended or may have one or more overhangs, e.g., 5′ and/or 3′ overhang(s). Nucleic acid modifications (e.g., base, sugar, and/or backbone modifications), non-standard nucleotides or nucleosides, etc., such as those known in the art as being useful in the context of RNA interference (RNAi), aptamer, or antisense-based molecules for research or therapeutic purposes may be incorporated in various embodiments. Such modifications may, for example, increase stability (e.g., by reducing sensitivity to cleavage by nucleases), decrease clearance in vivo, increase cell uptake, or confer other properties that improve the potency, efficacy, specificity, or otherwise render the nucleic acid more suitable for an intended use. Various non-limiting examples of nucleic acid modifications are described in, e.g., Deleavey G F, et al., Chemical modification of siRNA. Curr. Protoc. Nucleic Acid Chem. 2009; 39:16.3.1-16.3.22; Crooke, S T (ed.) Antisense drug technology: principles, strategies, and applications, Boca Raton: CRC Press, 2008; Kurreck, J. (ed.) Therapeutic oligonucleotides, RSC biomolecular sciences. Cambridge: Royal Society of Chemistry, 2008; U.S. Pat. Nos. 4,469,863; 5,536,821; 5,541,306; 5,637,683; 5,637,684; 5,700,922; 5,717,083; 5,719,262; 5,739,308; 5,773,601; 5,886,165; 5,929,226; 5,977,296; 6,140,482; 6,455,308 and/or in PCT application publications WO 00/56746 and WO 01/14398. Different modifications may be used in the two strands of a double-stranded nucleic acid. A nucleic acid may be modified uniformly or on only a portion thereof and/or may contain multiple different modifications. It will be appreciated that naturally-occurring allelic variants of the reference sequence for a particular nucleic acid or protein may exist in the population, and such variants may be used in certain embodiments. It will also be appreciated that variants arising due to alternative splicing may exist, which are encompassed herein in various embodiments.

“A “polypeptide” refers to a polymer of amino acids linked by peptide bonds. A protein is a molecule comprising one or more polypeptides. A peptide is a relatively short polypeptide, typically between about 2 and 100 amino acids (aa) in length, e.g., between 4 and 60 aa; between 8 and 40 aa; between 10 and 30 aa. The terms “protein”, “polypeptide”, and “peptide” may be used interchangeably. In general, a polypeptide may contain only standard amino acids or may comprise one or more non-standard amino acids (which may be naturally occurring or non-naturally occurring amino acids) and/or amino acid analogs in various embodiments. A “standard amino acid” is any of the 20 L-amino acids that are commonly utilized in the synthesis of proteins by mammals and are encoded by the genetic code. A “non-standard amino acid” is an amino acid that is not commonly utilized in the synthesis of proteins by mammals. Non-standard amino acids include naturally occurring amino acids (other than the 20 standard amino acids) and non-naturally occurring amino acids. In some embodiments, a non-standard, naturally occurring amino acid is found in mammals. For example, ornithine, citrulline, and homocysteine are naturally occurring non-standard amino acids that have important roles in mammalian metabolism. Exemplary non-standard amino acids include, e.g., singly or multiply halogenated (e.g., fluorinated) amino acids, D-amino acids, homo-amino acids, N-alkyl amino acids (other than proline), dehydroamino acids, aromatic amino acids (other than histidine, phenylalanine, tyrosine and tryptophan), and α,α disubstituted amino acids. An amino acid, e.g., one or more of the amino acids in a polypeptide, may be modified, for example, by addition, e.g., covalent linkage, of a moiety such as an alkyl group, an alkanoyl group, a carbohydrate group, a phosphate group, a lipid, a polysaccharide, a halogen, a linker for conjugation, a protecting group, etc. Modifications may occur anywhere in a polypeptide, e.g., the peptide backbone, the amino acid side-chains and the amino or carboxyl termini. A given polypeptide may contain many types of modifications. Polypeptides may be branched or they may be cyclic, with or without branching. Polypeptides may be conjugated with, encapsulated by, or embedded within a polymer or polymeric matrix, dendrimer, nanoparticle, microparticle, liposome, or the like. Modification may occur prior to or after an amino acid is incorporated into a polypeptide in various embodiments. Polypeptides may, for example, be purified from natural sources, produced in vitro or in vivo in suitable expression systems using recombinant DNA technology (e.g., by recombinant host cells or in transgenic animals or plants), synthesized through chemical means such as conventional solid phase peptide synthesis, and/or methods involving chemical ligation of synthesized peptides (see, e.g., Kent, S., J Pept Sci., 9(9):574-93, 2003 or U.S. Pub. No. 20040115774), or any combination of the foregoing. One of ordinary skill in the art will understand that a protein may be composed of a single amino acid chain or multiple chains associated covalently or noncovalently.

A “population of cells” can be a single cell or can comprise multiple cells in various embodiments. In some embodiments a population of cells comprises at least 10, 10², 10³, 10⁴, 10⁵, 10⁶, 10⁷, 10⁸, 10⁹, 10¹⁰, 10¹¹ cells, or more, or any range therebetween. In some embodiments of any relevant aspect herein a population of cells refers to multiple cells in a culture vessel such as a culture plate or dish. In some embodiments a population of cells refers to multiple cells exposed in parallel to the same agents or conditions. One of ordinary skill in the art will appreciate that a “population of cells” of a given type or having particular characteristic(s) comprises at least one cell of such type or having such characteristic(s) and may or may not further comprise one or more cells of different type(s) and/or lacking such characteristic(s). In various embodiments a population of cells is selected or purified to a desired level of uniformity or homogeneity with respect to type and/or characteristic(s). For example, in various embodiments a population of cells contains at least 10%, 20%, 30%, 40%, 50%, 60%, 70%, 80%, 90%, 95%, or more cells of such type and/or having such characteristic(s). It will be understood that many of the methods described herein are often practiced using populations of cells comprising multiple cells, e.g., in vitro or in vivo. Thus references to “a cell” should be understood as including embodiments in which the cell is a member of a population of cells. References to “cells” should be understood as including embodiments applicable to individual cells within a population comprising multiple cells and embodiments applicable to individual isolated cells. As will be understood by those of ordinary skill in the art, the number of members and/or one or more characteristic(s) of a population of cells may change over time, e.g., during a culture period. For example, at least some cells in the population may divide once or more and/or some cells may die. Hence, if a population of cells is maintained and/or subjected to one or more manipulations or steps, it should be understood that the population may have changed over time, and the term “population of cells” may thus refer to the population as it exists at the relevant time, e.g., the population resulting from the previous manipulation or step. It will also be appreciated that, in general, any manipulation or step performed on a population of cells may be performed on a subpopulation. For example, cells may be passaged, and only a portion of the cells retained for subsequent manipulation or steps, or a population may be divided into multiple aliquots, which may be used for different purposes.

“Purified” refers to agents that have been separated from most of the components with which they are associated in nature or when originally generated. In general, such purification involves action of the hand of man. Purified agents may be partially purified, substantially purified, or pure. Such agents may be, for example, at least 50%, 60%, 70%, 75%, 80%, 85%, 90%, 95%, 96%, 97%, 98%, 99%, or more than 99% pure. In some embodiments, a nucleic acid, polypeptide, or small molecule is purified such that it constitutes at least 75%, 80%, 85%, 90%, 95%, 96%, 97%, 98%, 99%, or more, of the total nucleic acid, polypeptide, or small molecule material, respectively, present in a preparation. In some embodiments, an organic substance, e.g., a nucleic acid, polypeptide, or small molecule, is purified such that it constitutes at least 75%, 80%, 85%, 90%, 95%, 96%, 97%, 98%, 99%, or more, of the total organic material present in a preparation. Purity may be based on, e.g., dry weight, size of peaks on a chromatography tracing (GC, HPLC, etc.), molecular abundance, electrophoretic methods, intensity of bands on a gel, spectroscopic data (e.g., NMR), elemental analysis, high throughput sequencing, mass spectrometry, or any art-accepted quantification method. In some embodiments, water, buffer substances, ions, and/or small molecules (e.g., synthetic precursors such as nucleotides or amino acids), can optionally be present in a purified preparation. A purified agent may be prepared by separating it from other substances (e.g., other cellular materials), or by producing it in such a manner to achieve a desired degree of purity. In some embodiments “partially purified” with respect to a molecule produced by a cell means that a molecule produced by a cell is no longer present within the cell, e.g., the cell has been lysed and, optionally, at least some of the cellular material (e.g., cell wall, cell membrane(s), cell organelle(s)) has been removed and/or the molecule has been separated or segregated from at least some molecules of the same type (protein, RNA, DNA, etc.) that were present in the lysate.

“RNA interference” (RNAi) encompasses processes in which a molecular complex known as an RNA-induced silencing complex (RISC) silences or “knocks down” gene expression in a sequence-specific manner in, e.g., eukaryotic cells, e.g., vertebrate cells, or in an appropriate in vitro system. RISC may incorporate a short nucleic acid strand (e.g., about 16-about 30 nucleotides (nt) in length) that pairs with and directs or “guides” sequence-specific degradation or translational repression of RNA (e.g., mRNA) to which the strand has complementarity. The short nucleic acid strand may be referred to as a “guide strand” or “antisense strand”. An RNA strand to which the guide strand has complementarity may be referred to as a “target RNA”. A guide strand may initially become associated with RISC components (in a complex sometimes termed the RISC loading complex) as part of a short double-stranded RNA (dsRNA), e.g., a short interfering RNA (siRNA). The other strand of the short dsRNA may be referred to as a “passenger strand” or “sense strand”. The complementarity of the structure formed by hybridization of a target RNA and the guide strand may be such that the strand can (i) guide cleavage of the target RNA in the RNA-induced silencing complex (RISC) and/or (ii) cause translational repression of the target RNA. Reduction of expression due to RNAi may be essentially complete (e.g., the amount of a gene product is reduced to background levels) or may be less than complete in various embodiments. For example, mRNA and/or protein level may be reduced by 50%, 60%, 70%, 75%, 80%, 85%, 90%, 95%, or more, in various embodiments. As known in the art, the complementarity between the guide strand and a target RNA need not be perfect (100%) but need only be sufficient to result in inhibition of gene expression. For example, in some embodiments 1, 2, 3, 4, 5, or more nucleotides of a guide strand may not be matched to a target RNA. “Not matched” or “unmatched” refers to a nucleotide that is mismatched (not complementary to the nucleotide located opposite it in a duplex, i.e., wherein Watson-Crick base pairing does not take place) or forms at least part of a bulge. Examples of mismatches include, without limitation, an A opposite a G or A, a C opposite an A or C, a U opposite a C or U, a G opposite a G. A bulge refers to a sequence of one or more nucleotides in a strand within a generally duplex region that are not located opposite to nucleotide(s) in the other strand. “Partly complementary” refers to less than perfect complementarity. In some embodiments a guide strand has at least about 80%, 85%, or 90%, e.g., least about 91%, 92%, 93%, 94%, 95%, 96%, 97%, 98%, 99%, or 100% sequence complementarity to a target RNA over a continuous stretch of at least about 15 nt, e.g., between 15 nt and 30 nt, between 17 nt and 29 nt, between 18 nt and 25 nt, between 19 nt and 23 nt, of the target RNA. In some embodiments at least the seed region of a guide strand (the nucleotides in positions 2-7 or 2-8 of the guide strand) is perfectly complementary to a target RNA. In some embodiments, a guide strand and a target RNA sequence may form a duplex that contains no more than 1, 2, 3, or 4 mismatched or bulging nucleotides over a continuous stretch of at least 10 nt, e.g., between 10-30 nt. In some embodiments a guide strand and a target RNA sequence may form a duplex that contains no more than 1, 2, 3, 4, 5, or 6 mismatched or bulging nucleotides over a continuous stretch of at least 12 nt, e.g., between 10-30 nt. In some embodiments, a guide strand and a target RNA sequence may form a duplex that contains no more than 1, 2, 3, 4, 5, 6, 7, or 8 mismatched or bulging nts over a continuous stretch of at least 15 nt, e.g., between 10-30 nt. In some embodiments, a guide strand and a target RNA sequence may form a duplex that contains no mismatched or bulging nucleotides over a continuous stretch of at least 10 nt, e.g., between 10-30 nt. In some embodiments, between 10-30 nt is 10, 11, 12, 13, 14, 15, 16, 17, 18, 19, 20, 21, 22, 23, 24, 25, 26, 27, 28, 29, or 30 nt.

“Progenitor cell” as used herein encompasses stem cells as described herein as well as cells that may have a more limited self-renewal ability or may typically not self-renew but instead divide to give rise to two daughters that may be identical to the mother progenitor cell or may become more differentiated than the mother cell and/or more lineage-restricted than the mother cell. Certain progenitor cells may occupy an intermediate position in a cell lineage, between a stem cell and a more differentiated cell. Such a progenitor cell may be more differentiated than a stem cell from which it arose and/or may generate daughter(s) that are more differentiated than itself.

As used herein, the term “RNAi agent” encompasses nucleic acids that can be used to achieve RNAi in eukaryotic cells. Short interfering RNA (siRNA), short hairpin RNA (shRNA), and microRNA (miRNA) are examples of RNAi agents. siRNAs typically comprise two separate nucleic acid strands that are hybridized to each other to form a structure that contains a double stranded (duplex) portion at least 15 nt in length, e.g., about 15-about 30 nt long, e.g., between 17-27 nt long, e.g., between 18-25 nt long, e.g., between 19-23 nt long, e.g., 15, 16, 17, 18, 19, 20, 21, 22, 23, 24, 25, 26, 27, 28, 29, or 30 nucleotides. In some embodiments the strands of an siRNA are perfectly complementary to each other within the duplex portion. In some embodiments the duplex portion may contain one or more unmatched nucleotides, e.g., one or more mismatched (non-complementary) nucleotide pairs or bulged nucleotides. In some embodiments either or both strands of an siRNA may contain up to about 1, 2, 3, or 4 unmatched nucleotides within the duplex portion. In some embodiments a strand may have a length of between 15-35 nt, e.g., between 17-29 nt, e.g., 19-25 nt, e.g., 21-23 nt. Strands may be equal in length or may have different lengths in various embodiments. In some embodiments strands may differ by between 1-10 nt in length. A strand may have a 5′ phosphate group and/or a 3′ hydroxyl (—OH) group. Either or both strands of an siRNA may comprise a 3′ overhang of, e.g., about 1-10 nt (e.g., 1-5 nt, e.g., 2 nt). Overhangs may be the same length or different in lengths in various embodiments. In some embodiments an overhang may comprise or consist of deoxyribonucleotides, ribonucleotides, or modified nucleotides or modified ribonucleotides such as 2′-O-methylated nucleotides, or 2′-O-methyl-uridine. An overhang may be perfectly complementary, partly complementary, or not complementary to a target RNA in a hybrid formed by the guide strand and the target RNA in various embodiments.

shRNAs are nucleic acid molecules that comprise a stem-loop structure and a length typically between about 40-150 nt, e.g., about 50-100 nt, e.g., 60-80 nt. A “stem-loop structure” (also referred to as a “hairpin” structure) refers to a nucleic acid having a secondary structure that includes a region of nucleotides which are known or predicted to form a double strand (stem portion; duplex) that is linked on one side by a region of (usually) predominantly single-stranded nucleotides (loop portion). Such structures are well known in the art and the term is used consistently with its meaning in the art. A guide strand sequence may be positioned in either arm of the stem, i.e., 5′ with respect to the loop or 3′ with respect to the loop in various embodiments. As is known in the art, the stem structure does not require exact base-pairing (perfect complementarity). Thus, the stem may include one or more unmatched residues or the base-pairing may be exact, i.e., it may not include any mismatches or bulges. In some embodiments the stem is between 15-30 nt, e.g., between 17-29 nt, e.g., 19-25 nt. In some embodiments the stem is between 15-19 nt. In some embodiments the stem is between 19-30 nt. The primary sequence and number of nucleotides within the loop may vary. Examples of loop sequences include, e.g., UGGU; ACUCGAGA; UUCAAGAGA. In some embodiments a loop sequence found in a naturally occurring miRNA precursor molecule (e.g., a pre-miRNA) may be used. In some embodiments a loop sequence may be absent (in which case the termini of the duplex portion may be directly linked). In some embodiments a loop sequence may be at least partly self-complementary. In some embodiments the loop is between 1 and 20 nt in length, e.g., 1-15 nt, e.g., 4-9 nt. The shRNA structure may comprise a 5′ or 3′ overhang. As known in the art, an shRNA may undergo intracellular processing, e.g., by the ribonuclease (RNase) III family enzyme known as Dicer, to remove the loop and generate an siRNA.

Mature endogenous miRNAs are short (typically 18-24 nt, e.g., about 22 nt), single-stranded RNAs that are generated by intracellular processing from larger, endogenously encoded precursor RNA molecules termed miRNA precursors (see, e.g., Bartel, D., MicroRNAs: genomics, biogenesis, mechanism, and function. Cell. 116(2):281-97 (2004); Bartel D P. MicroRNAs: target recognition and regulatory functions. Cell. 136(2):215-33 (2009); Winter, 3., et al., Nature Cell Biology 11: 228-234 (2009), Artificial miRNA may be designed to take advantage of the endogenous RNAi pathway in order to silence a target RNA of interest.

An RNAi agent that contains a strand sufficiently complementary to an RNA of interest so as to result in reduced expression of the RNA of interest (e.g., as a result of degradation or repression of translation of the RNA) in a cell or in an in vitro system capable of mediating RNAi and/or that comprises a sequence that is at least 80%, 90%, 95%, or more (e.g., 100%) complementary to a sequence comprising at least 10, 12, 15, 17, or 19 consecutive nucleotides of an RNA of interest may be referred to as being “targeted to” the RNA of interest. An RNAi agent targeted to an RNA transcript may also considered to be targeted to a gene from which the transcript is transcribed. In some embodiments an RNAi agent is a vector (e.g., an expression vector) suitable for causing intracellular expression of one or more transcripts that give rise to a siRNA, shRNA, or miRNA in the cell. Such a vector may be referred to as an “RNAi vector”. An RNAi vector may comprise a template that, when transcribed, yields transcripts that may form a siRNA (e.g., as two separate strands that hybridize to each other), shRNA, or miRNA precursor (e.g., pri-miRNA or pre-mRNA). An RNAi agent may be produced in any of variety of ways in various embodiments. For example, nucleic acid strands may be chemically synthesized (e.g., using standard nucleic acid synthesis techniques) or may be produced in cells or using an in vitro transcription system. Strands may be allowed to hybridize (anneal) in an appropriate liquid composition (sometimes termed an “annealing buffer”). An RNAi vector may be produced using standard recombinant nucleic acid techniques.

A “sample” as used herein can be any biological specimen that contains one or more cell(s), tissue, or cellular material (e.g., cell lysate or fraction thereof). In some embodiments a sample is obtained from (i.e., originates from, was initially removed from) a subject. Methods of obtaining such samples are known in the art and include, e.g., tissue biopsy such as excisional biopsy, incisional biopy, or core biopsy; fine needle aspiration biopsy; brushings; lavage; or collecting body fluids such as blood, sputum, lymph, mucus, saliva, urine, etc., etc. In many embodiments, a sample contains at least some intact cells at the time it is removed from a subject. In some embodiments a sample retains at least some of the tissue microarchitecture present in the tissue prior to removal. A sample may be subjected to one or more processing steps after having been obtained from a subject and/or may be split into one or more portions, which may entail removing or discarding part of the original sample. The term “sample” encompasses such processed samples, portions of samples, etc., and such samples are considered to have been obtained from the subject from whom the initial sample was removed. In some embodiments, a sample has been obtained or is obtained from an individual who is apparently healthy, e.g., the subject has not been diagnosed with a disease, e.g., cancer, and is not suspected of having a disease, e.g., cancer, at the time the sample is obtained. In some embodiments, a sample has been obtained or is obtained from an individual who has been diagnosed with cancer or is at increased risk of cancer, is suspected of having cancer, or is at risk of cancer recurrence. In some embodiments a sample has been obtained or is obtained from a tumor prior to or after removal of the tumor from a subject. A sample used in a method described herein may have been procured directly from a subject or procured indirectly, e.g., by receiving the sample through a chain of one or more persons originating with a person who procured the sample directly from the subject, e.g., by performing a biopsy or other procedure on the subject. A “tumor sample” is a sample that includes at least some cells, tissue, or cellular material obtained from a tumor. In some embodiments the sample comprises tumor cells. In some embodiments the sample comprises tumor tissue. In some embodiments if a tumor sample comprises areas of neoplastic tissue and areas of non-neoplastic tissue (e.g., as identified using standard histopathological criteria), an assessment or score can be based on assessing neoplastic tissue. Non-neoplastic tissue may be used as a control.

A “small molecule” as used herein, is an organic molecule that is less than about 2 kilodaltons (KDa) in mass. In some embodiments, the small molecule is less than about 1.5 KDa, or less than about 1 KDa. In some embodiments, the small molecule is less than about 800 daltons (Da), 600 Da, 500 Da, 400 Da, 300 Da, 200 Da, or 100 Da. Often, a small molecule has a mass of at least 50 Da. In some embodiments, a small molecule is non-polymeric. In some embodiments, a small molecule is not an amino acid. In some embodiments, a small molecule is not a nucleotide. In some embodiments, a small molecule is not a saccharide. In some embodiments, a small molecule contains multiple carbon-carbon bonds and, in some embodiments, comprises one or more heteroatoms and/or one or more functional groups important for structural interaction with proteins (e.g., hydrogen bonding), e.g., an amine, carbonyl, hydroxyl, or carboxyl group, and in some embodiments at least two functional groups. Small molecules often comprise one or more cyclic carbon or heterocyclic structures and/or aromatic or polyaromatic structures, optionally substituted with one or more of the above functional groups.

“Stem cell” refers to a cell that is (a) relatively undifferentiated; (b) capable of generating daughter cells (“daughters”) that are similarly undifferentiated; (c) capable of generating a lineage of such daughters that are able to reproduce themselves over a large number of successive growth-and-division cycles (also termed “self-renewal”); and (d) capable of generating daughters that are able, under appropriate conditions, to enter into a program of differentiation that enables such cells to acquire the specialized traits of one or another functional tissue in the mammalian body. In some aspects, a stem cell is capable of dividing asymmetrically, thereby generating two daughter cells that are unequal to one another, because one of the daughter cells retains the phenotypic state of the mother stem cells while the other daughter cell enters into a new state of differentiation, such as the differentiated state of a progenitor cell. The term “adult stem cell”, also referred to as a “somatic stem cell” refers to stem cells that can be found in or isolated from a mammalian organism after early embryonic development and are not germ cells. As known in the art, adult stem cells can be found in fetuses and juveniles as well as adults. In some embodiments adult stem cells have multi-lineage potential. In some embodiments adult stem cells have single-lineage potential. As used herein, “adult stem cell” encompasses somatic cells that are generated or derived in culture from a somatic cell (e.g., a somatic cell that is more differentiated than the stem cell) and that have the properties of a stem cell but are not pluripotent (as judged by art-accepted means of assessing pluripotency, such as teratoma formation assays or expression of particular markers characteristic of pluripotent cells) or totipotent. For example, such cells may not give rise to cells of all three germ layers when introduced into suitable non-human animal hosts, as would be the case for a pluripotent cell.

A “subject” may be any vertebrate organism in various embodiments. Typically a subject is a mammal. In some embodiments a subject is an individual to whom an agent is administered, e.g., for experimental, diagnostic, and/or therapeutic purposes or from whom a sample is obtained or on whom a procedure is performed. In some embodiments a subject is a human, non-human primate, rodent (e.g., mouse, rat, rabbit), ungulate (e.g., ovine, bovine, equine, caprine species), canine, or feline. In some embodiments, a subject is an adult. For purposes hereof a human at least 18 years of age is considered an adult.

“Treat”, “treating” and similar terms refer to providing medical and/or surgical management of a subject. Treatment can include, but is not limited to, administering an agentor composition (e.g., a pharmaceutical composition) to a subject. Treatment is typically undertaken in an effort to alter the course of a disease, disorder, or undesirable condition in a manner beneficial to the subject. The effect of treatment can generally include reversing, alleviating, reducing severity of, delaying the onset of, curing, inhibiting the progression of, and/or reducing the likelihood of occurrence or reoccurence of the disease, disorder, or condition to which such term applies, or one or more symptoms or manifestations of such disease, disorder or condition. In some embodiments an agent or composition is administered to a subject who has developed a disease or condition or is at increased risk of doing so relative to a member of the general population. In some embodiments an agent or composition is administered prophylactically, i.e., before development of any symptom or manifestation of a condition. Typically in this case the subject will be at risk of developing the condition. It will be understood that “administering” encompasses self-administration. “Preventing” can refer to administering an agent or composition (e.g., a pharmaceutical composition) to a subject who has not developed a disease or condition, so as to reduce the likelihood that the disease or condition will occur or so as to reduce the severity of the disease or condition should it occur. The subject may be identified as at risk of developing the disease or condition (e.g., at increased risk relative to many most other members of the population (who may be matched with respect to various demographic factors such as age, sex, ethnicity, etc.) or as having one or more risk factors that increases likelihood of developing the disease or condition).

A “variant” of a particular polypeptide or polynucleotide has one or more alterations (e.g., additions, substitutions, and/or deletions, which may be referred to collectively as “mutations”) with respect to the polypeptide or polynucleotide, which may be referred to as the “original polypeptide” or “original polynucleotide”, respectively. An addition may be an insertion or may be at either terminus. A variant may be shorter or longer than the original polypeptide or polynucleotide. The term “variant” encompasses “fragments”. A “fragment” is a continuous portion of a polypeptide or polynucleotide that is shorter than the original polypeptide. In some embodiments a variant comprises or consists of a fragment. In some embodiments a fragment or variant is at least 20%, 30%, 40%, 50%, 60%, 70%, 80%, 90%, 95%, 96%, 97%, 98%, 99%, or more as long as the original polypeptide or polynucleotide. A fragment may be an N-terminal, C-terminal, or internal fragment. In some embodiments a variant polypeptide comprises or consists of at least one domain of an original polypeptide. In some embodiments a variant polynucleotide hybridizes to an original polynucleotide under stringent conditions, e.g., high stringency conditions, for sequences of the length of the original polypeptide. In some embodiments a variant polypeptide or polynucleotide comprises or consists of a polypeptide or polynucleotide that is at least 50%, 60%, 70%, 80%, 90%, 95%, 96%, 97%, 98%, 99%, or more identical in sequence to the original polypeptide or polynucleotide over at least 20%, 30%, 40%, 50%, 60%, 70%, 80%, 90%, 95%, 96%, 97%, 98%, 99%, or 100% of the original polypeptide or polynucleotide. In some embodiments a variant polypeptide comprises or consists of a polypeptide that is at least 50%, 60%, 70%, 80%, 90%, 95%, 96%, 97%, 98%, 99%, or more identical in sequence to the original polypeptide over at least 20%, 30%, 40%, 50%, 60%, 70%, 80%, 90%, 95%, 96%, 97%, 98%, 99%, or 100% of the original polypeptide, with the proviso that, for purposes of computing percent identity, a conservative amino acid substitution is considered identical to the amino acid it replaces. In some embodiments a variant polypeptide comprises or consists of a polypeptide that is at least 50%, 60%, 70%, 80%, 90%, 95%, 96%, 97%, 98%, 99%, or more identical to the original polypeptide over at least 20%, 30%, 40%, 50%, 60%, 70%, 80%, 90%, 95%, 96%, 97%, 98%, 99%, or 100% of the original polypeptide, with the proviso that any one or more amino acid substitutions (up to the total number of such substitutions) may be restricted to conservative substitutions. In some embodiments a percent identity is measured over at least 100; 200; 300; 400; 500; 600; 700; 800; 900; 1,000; 1,200; 1,500; 2,000; 2,500; 3,000; 3,500; 4,000; 4,500; or 5,000 amino acids. In some embodiments the sequence of a variant polypeptide comprises or consists of a sequence that has N amino acid differences with respect to an original sequence, wherein N is any integer between 1 and 10 or between 1 and 20 or any integer up to 1%, 2%, 5%, or 10% of the number of amino acids in the original polypeptide, where an “amino acid difference” refers to a substitution, insertion, or deletion of an amino acid. In some embodiments a difference is a conservative substitution. Conservative substitutions may be made, e.g., on the basis of similarity in side chain size, polarity, charge, solubility, hydrophobicity, hydrophilicity and/or the amphipathic nature of the residues involved. In some embodiments, conservative substitutions may be made according to Table A, wherein amino acids in the same block in the second column and in the same line in the third column may be substituted for one another other in a conservative substitution. Certain conservative substitutions are substituting an amino acid in one row of the third column corresponding to a block in the second column with an amino acid from another row of the third column within the same block in the second column.

TABLE A

| | | |
|---|---|---|
| Aliphatic | Nonpolar | G A P |
| | | I L V |
| | Polar-uncharged | C S T M |
| | | N Q |
| | Polar-charged | D E |
| | | K R |
| Aromatic | | H F W Y |

In some embodiments, proline (P) is considered to be in an individual group. In some embodiments, cysteine (C) is considered to be in an individual group. In some embodiments, proline (P) and cysteine (C) are each considered to be in an individual group.

In some embodiments a variant is a functional variant, i.e., the variant at least in part retains at least one activity of the original polypeptide or polynucleotide. In some embodiments a variant at least in part retains more than one or substantially all known biologically significant activities of the original polypeptide or polynucleotide. An activity may be, e.g., a catalytic activity, binding activity, ability to perform or participate in a biological function or process, etc. In some embodiments an activity of a variant may be at least 10%, 20%, 30%, 40%, 50%, 60%, 70%, 80%, 90%, 95%, or more, of the activity of the original polypeptide or polynucleotide, up to approximately 100%, approximately 125%, or approximately 150% of the activity of the original polypeptide or polynucleotide, in various embodiments. In some embodiments a variant, e.g., a functional variant, comprises or consists of a polypeptide at least 95%, 96%, 97%, 98%, 99%. 99.5% or 100% identical to an original polypeptide or polynucleotide over at least 70%, 75%, 80%, 85%, 90%, 95%, 96%, 97%, 98%, or 99% or 100% of the original polypeptide or polynucleotide. In some embodiments an alteration, e.g., a substitution or deletion, e.g., present in a functional variant (as compared with the original polynucleotide or polypeptide), does not alter or delete an amino acid or nucleotide that is known or predicted to be important for an activity, e.g., a known or predicted catalytic residue or a residue involved in binding a substrate or cofactor or receptor or ligand or a site whose post-translational modification is important for activity or normal localization. In some embodiments an alteration, e.g., a substitution or deletion does not alter or delete an amino acid or nucleotide important for a protein-protein interaction (e.g., dimerization) or protein-nucleic acid (e.g., protein-DNA) binding or normal localization. In some embodiments nucleotide(s), amino acid(s), or region(s) exhibiting lower degrees of conservation across species as compared with other amino acids or regions may be selected for alteration. As will be understood, variants can be created by introducing one or more nucleotide alterations, e.g., one or more substitution(s), addition(s) and/or deletion(s) into a nucleotide sequence encoding a polypeptide, such that one or more amino acid alterations, e.g., substitution(s), addition(s) and/or deletion(s) are introduced into the encoded polypeptide. One of skill in the art can readily generate functional variants or fragments of polypeptides of interest herein. Alterations can be introduced by standard techniques, such as site-directed mutagenesis, PCR-mediated mutagenesis, etc. Variants may be tested in one or more suitable assays to assess activity.

A “vector” may be any of a number of nucleic acid molecules or viruses or portions thereof that are capable of mediating entry of, e.g., transferring, transporting, etc., a nucleic acid of interest between different genetic environments or into a cell. The nucleic acid of interest may be linked to, e.g., inserted into, the vector using, e.g., restriction and ligation. Vectors include, for example, DNA or RNA plasmids, cosmids, naturally occurring or modified viral genomes or portions thereof, nucleic acids that can be packaged into viral capsids, mini-chromosomes, artificial chromosomes, etc. Plasmid vectors typically include an origin of replication (e.g., for replication in prokaryotic cells). A plasmid may include part or all of a viral genome (e.g., a viral promoter, enhancer, processing or packaging signals, and/or sequences sufficient to give rise to a nucleic acid that can be integrated into the host cell genome and/or to give rise to infectious virus). Viruses or portions thereof that can be used to introduce nucleic acids into cells may be referred to as viral vectors. Viral vectors include, e.g., adenoviruses, adeno-associated viruses, retroviruses (e.g., lentiviruses), vaccinia virus and other poxviruses, herpesviruses (e.g., herpes simplex virus), and others. Viral vectors may or may not contain sufficient viral genetic information for production of infectious virus when introduced into host cells, i.e., viral vectors may be replication-competent or replication-defective. In some embodiments, e.g., where sufficient information for production of infectious virus is lacking, it may be supplied by a host cell or by another vector introduced into the cell, e.g., if production of virus is desired. In some embodiments such information is not supplied, e.g., if production of virus is not desired. A nucleic acid to be transferred may be incorporated into a naturally occurring or modified viral genome or a portion thereof or may be present within a viral capsid as a separate nucleic acid molecule. A vector may contain one or more nucleic acids encoding a marker suitable for identifying and/or selecting cells that have taken up the vector. Markers include, for example, various proteins that increase or decrease either resistance or sensitivity to antibiotics or other agents (e.g., a protein that confers resistance to an antibiotic such as puromycin, hygromycin or blasticidin), enzymes whose activities are detectable by assays known in the art (e.g., β-galactosidase or alkaline phosphatase), and proteins or RNAs that detectably affect the phenotype of cells that express them (e.g., fluorescent proteins). Vectors often include one or more appropriately positioned sites for restriction enzymes, which may be used to facilitate insertion into the vector of a nucleic acid, e.g., a nucleic acid to be expressed. An expression vector is a vector into which a desired nucleic acid has been inserted or may be inserted such that it is operably linked to regulatory elements (also termed “regulatory sequences”, “expression control elements”, or “expression control sequences”) and may be expressed as an RNA transcript (e.g., an mRNA that can be translated into protein or a noncoding RNA such as an shRNA or miRNA precursor). Expression vectors include regulatory sequence(s), e.g., expression control sequences, sufficient to direct transcription of an operably linked nucleic acid under at least some conditions; other elements required or helpful for expression may be supplied by, e.g., the host cell or by an in vitro expression system. Such regulatory sequences typically include a promoter and may include enhancer sequences or upstream activator sequences. In some embodiments a vector may include sequences that encode a 5′ untranslated region and/or a 3′ untranslated region, which may comprise a cleavage and/or polyadenylation signal. In general, regulatory elements may be contained in a vector prior to insertion of a nucleic acid whose expression is desired or may be contained in an inserted nucleic acid or may be inserted into a vector following insertion of a nucleic acid whose expression is desired. As used herein, a nucleic acid and regulatory element(s) are said to be “operably linked” when they are covalently linked so as to place the expression or transcription of the nucleic acid under the influence or control of the regulatory element(s). For example, a promoter region would be operably linked to a nucleic acid if the promoter region were capable of effecting transcription of that nucleic acid. One of ordinary skill in the art will be aware that the precise nature of the regulatory sequences useful for gene expression may vary between species or cell types, but may in general include, as appropriate, sequences involved with the initiation of transcription, RNA processing, or initiation of translation. The choice and design of an appropriate vector and regulatory element(s) is within the ability and discretion of one of ordinary skill in the art. For example, one of ordinary skill in the art will select an appropriate promoter (or other expression control sequences) for expression in a desired species (e.g., a mammalian species) or cell type. A vector may contain a promoter capable of directing expression in mammalian cells, such as a suitable viral promoter, e.g., from a cytomegalovirus (CMV), retrovirus, simian virus (e.g., SV40), papilloma virus, herpes virus or other virus that infects mammalian cells, or a mammalian promoter from, e.g., a gene such as EF1alpha, ubiquitin (e.g., ubiquitin B or C), globin, actin, phosphoglycerate kinase (PGK), etc., or a composite promoter such as a CAG promoter (combination of the CMV early enhancer element and chicken beta-actin promoter). In some embodiments a human promoter may be used. In some embodiments, a promoter that ordinarily directs transcription by a eukaryotic RNA polymerase I (a “pol I promoter”), e.g., (a U6, H1, 7SK or tRNA promoter or a functional variant thereof) may be used. In some embodiments, a promoter that ordinarily directs transcription by a eukaryotic RNA polymerase II (a “pol II promoter”) or a functional variant thereof is used. In some embodiments, a promoter that ordinarily directs transcription by a eukaryotic RNA polymerase III (a “pol III promoter”), e.g., a promoter for transcription of ribosomal RNA (other than 5S rRNA) or a functional variant thereof is used. One of ordinary skill in the art will select an appropriate promoter for directing transcription of a sequence of interest. Examples of expression vectors that may be used in mammalian cells include, e.g., the pcDNA vector series, pSV2 vector series, pCMV vector series, pRSV vector series, pEF1 vector series, Gateway® vectors, etc. Examples of virus vectors that may be used in mammalian cells include, e.g., adenoviruses, adeno-associated viruses, poxviruses such as vaccinia viruses and attenuated poxviruses, retroviruses (e.g., lentiviruses), Semliki Forest virus, Sindbis virus, etc. In some embodiments, regulatable (e.g., inducible or repressible) expression control element(s), e.g., a regulatable promoter, is/are used so that expression can be regulated, e.g., turned on or increased or turned off or decreased. For example, the tetracycline-regulatable gene expression system (Gossen & Bujard, Proc. Natl. Acad. Sci. 89:5547-5551, 1992) or variants thereof (see, e.g., Allen. N, et al. (2000) Mouse Genetics and Transgenics: 259-263; Urlinger, S, et al. (2000). Proc. Natl. Acad. Sci. U.S.A. 97 (14): 7963-8; Zhou, X., et al (2006). Gene Ther. 13 (19): 1382-1390 for examples) can be employed to provide inducible or repressible expression. Other inducible/repressible systems may be used in various embodiments. For example, expression control elements that can be regulated by small molecules such as artificial or naturally occurring hormone receptor ligands (e.g., steroid receptor ligands such as naturally occurring or synthetic estrogen receptor or glucocorticoid receptor ligands), tetracycline or analogs thereof (e.g., doxycycline), metal-regulated systems (e.g., metallothionein promoter) may be used in certain embodiments. In some embodiments, tissue-specific or cell type specific regulatory element(s) are used, e.g., in order to direct expression in one or more selected tissues or cell types. In some embodiments a vector comprises a polynucleotide sequence that encodes a polypeptide, wherein the polynucleotide sequence is positioned in frame with a nucleic acid inserted into the vector so that an N- or C-terminal fusion is created. In some embodiments the polypeptide encoded by the polynucleotide sequence is a targeting peptide. A targeting peptide may comprise a signal sequence (which directs secretion of a protein) or a sequence that directs the expressed protein to a specific organelle or location in the cell such as the nucleus or mitochondria. In some embodiments the polypeptide comprises a tag. In some embodiments a tag is useful to facilitate detection and/or purification of a protein that contains it. Examples of tags include polyhistidine-tag (e.g., 6×-His tag), glutathione-S-transferase, maltose binding protein, NUS tag, SNUT tag, Strep tag, epitope tags such as V5, HA, Myc, or FLAG. In some embodiments a protease cleavage site is located in the region between the protein encoded by the inserted nucleic acid and the polypeptide, allowing the polypeptide to be removed by exposure to the protease.

### II. Cooperation with EMT in Determining Stem Cell State and Methods Relating Thereto

Stem cells play important roles in development, tissue repair and regeneration, and other biological processes in mammalian organisms. In addition stem cells hold great interest as sources of cells for use in a variety of applications. Adult stem cells that reside in or are obtained from a particular tissue or organ (or portion thereof) are typically capable of giving rise to one or more cell lineages culminating in one or more differentiated cell types characteristic of that tissue or organ. For example, mammary stem cells can give rise to a mammary cell lineage that leads to differentiated luminal mammary epithelial cells and a lineage that lead to differentiated mammary myoepithelial cells. Adult stem cells provide a source of cells for repair or regeneration or physiological growth of various tissues or organs during life. Stem cells also play a role in tumorigenesis. Cancer cells in a tumor are typically functionally heterogeneous and, as is the case with normal tissues, are often organized in a hierarchical manner. Cancer stem cells (CSCs) can be defined functionally as those cells within a tumor that have the capacity to seed and generate secondary tumors, e.g., with high efficiency. This CSC state may be manifested by the ability of a CSC to seed a new tumor following implantation into an appropriate mouse host or, during the natural course of malignant progression, disseminate from a primary tumor and seed a new colony of tumor cells at a distant anatomical site, the latter colony being termed a metastasis. These highly tumorigenic cells, also referred to as “tumor-initiating cells”, undergo self-renewal and can also generate weakly tumorigenic or non-tumorigenic cancer cells. CSCs thus possess characteristics associated with normal stem cells, such as self-renewal ability and the ability to give rise to cells that differ phenotypically from themselves. CSCs are widely considered to play a major role in driving tumor growth and progression.

As used herein, the term “epithelial to mesenchymal transition” (EMT), refers to a transformation, or partial transformation, of an epithelial state of cell differentiation (“epithelial characteristics” or “epithelial properties”) into a cell having one or more characteristics of cells residing in a mesenchymal state of cell differentiation (“mesenchymal characteristics” or “mesenchmal properties”). The EMT is widely documented to play an important role in converting normal and neoplastic epithelial cells into cells with a more mesenchymal phenotype. Most epithelial cells typically are closely attached to one another by intercellular adhesion complexes (e.g., tight junctions, adherens junctions, desmosomes, gap junctions) in their lateral membranes, typically tend to grow in clusters or sheets (layers), express characteristic epithelial markers such as E-cadherin and cytokeratins, and have relatively low or absent expression of mesenchymal markers such as N-cadherin, fibronectin, and vimentin. Mesenchymal properties include, e.g., a relative lack of intercellular junctions, more elongated shape, greater tendency to exist as single cells rather than in clusters as compared with epithelial cells, expression of characteristic mesenchymal markers such as vimentin, fibronectin, and N-cadherin, increased migratory ability as compared with epithelial cells, and relatively low or absent expression of epithelial markers such as E-cadherin, and cytokeratins. An epithelial cell that has undergone EMT may exhibit one or more of such mesenchymal properties thus epithelial cells, both normal and neoplastic, may undergo a partial EMT and acquire a subset of mesenchymal characteristics while retaining preexisting epithelial characteristics; alternatively an epithelial cell, both normal and neoplastic, may undergo a complete EMT, and thus shed all preexisting distinctively epithelial characteristics and acquire a suite of characteristically mesenchymal attributes. In the context of neoplasia, passage of tumor cells through an EMT can result in the acquisition of cell-biological traits associated with high-grade malignancy, such as motility, invasiveness, and an increased resistance to apoptosis. In addition to conferring mesenchymal traits, normal and neoplastic adult epithelial cells that are induced to pass through an EMT can acquire properties associated with normal stem cells (SCs) and cancer stem cells (CSCs).

In some aspects, the present disclosure relates to the Applicants' identification of a genetic pathway that can cooperate with the EMT to promote formation of stem cells, e.g., adult stem cells, from epithelial cells or to maintain stem cells in a stem cell state. Among other things, the disclosure provides the recognition that the EMT program can cooperate with gene expression programs mediated by certain transcription factors (TFs) that are expressed in stem cells and/or that are expressed in early developmental processes, to convert differentiated epithelial cells into stem cells or to maintain stem cells in the stem cell state. In some embodiments the disclosure relates to the discovery that certain TFs can cooperate with the EMT to promote formation of SCs from differentiated epithelial cells or to maintain SCs in a SC state. Without wishing to be bound by any theory, it is proposed that expression of such TFs activates a complementary, distinct cell-biological program that cooperates with the EMT program to enable entrance into the SC state. In some embodiments, the activation of certain genetic pathways in a population of epithelial cells in combination with inducing EMT generates substantially more stem cells than would result from either (i) inducing EMT and not activating the genetic pathway or (ii) activating the genetic pathway and not inducing EMT. For example, in some embodiments the combination of inducing EMT and activating a cooperating genetic pathway results in the formation of at least 5, 10, 25, or more times as many SCs as does either manipulation performed individually.

Cells that have undergone an EMT typically express one or more transcription factors (TFs) that are able to induce EMT in epithelial cells. TFs that can induce EMT in at least some epithelial cell types are sometimes referred to herein as EMT-TFs. EMT-TFs are normally expressed transiently during certain steps of embryonic morphogenesis, during wound healing, during certain types of inflammation, in certain stem cells, and in certain high-grade tumors. EMT-TFs include, e.g., Slug, Snail, Twist1, Twist2, Zeb1, Zeb2, Goosecoid, FoxC2, Tcf3, Klf8, FoxC1, FoxQ1, Six1, Lbx1, Yap1, and HIF-1. In some embodiments the disclosure relates to the discovery that certain other TFs can cooperate with EMT-TFs to promote formation of SCs from epithelial cells or to maintain SCs in a SC state. In some embodiments EMT is brought about by causing an epithelial cell to express an EMT-TF. As described further below, in some embodiments expression of an EMT-TF is achieved by introducing a nucleic acid encoding a polypeptide comprising the EMT-TF into a cell or an ancestor of the cell. In some embodiments EMT is brought about by modulation of one or more endogenous signaling pathways. An agent that can induce an epithelial cell to undergo EMT I sometimes referred to herein as an “EMT-inducing agent”. An agent that can cooperate with EMT and/or with an EMT-inducing agent to promote (increase) generation of SCs from one or more epithelial cell types or to maintain SCs in an SC state is sometimes referred to herein as an “EMT-cooperating agent”. In some embodiments an EMT-cooperating agent comprises an EMT-cooperating TF, which term refers to a TF that can cooperate with EMT and/or with an EMT-inducing agent to promote generation of SCs from one or more epithelial cell types or to maintain SCs in an SC state. In some embodiments an EMT-cooperating agent comprises a nucleic acid that encodes a polypeptide comprising an EMT-TF. In most embodiments an EMT-cooperating TF is not itself an EMT-TF, at least as used in a method, product, or composition described herein, e.g., the EMT-cooperating TF does not have appreciable ability to induce EMT by itself.

In some embodiments, a method of generating stem cells from epithelial cells comprises steps of: (a) providing a population of epithelial cells; and (b) inducing epithelial-mesenchymal transition (EMT) and exposing the population of epithelial cells to an EMT-cooperating agent, thereby generating stem cells in the population. In some embodiments, a method of generating stem cells from epithelial cells comprises steps of: (a) providing a population of epithelial cells; and (b) inducing epithelial-mesenchymal transition (EMT) and increasing the amount or activity of at least one EMT-cooperating TF in the population of epithelial cells, thereby generating stem cells in the population. In some embodiments, a method of generating stem cells from epithelial cells comprises steps of: (a) providing a population of epithelial cells; and (b) increasing the amount or activity of at least one EMT-TF and increasing the amount or activity of at least one EMT-cooperating TF in the population of epithelial cells, thereby generating stem cells in the population.

The phrase “exposing cells” is used interchangeably with “contacting cells” herein. In certain embodiments exposing cell(s) to an agent in vitro comprises adding an agent to a culture medium or culture vessel in which the cells are maintained or adding cell(s) to culture medium containing the agent. In certain embodiments exposing cell(s) to an agent in vivo comprises administering the agent to a subject. It will be understood that the precise time of exposure may begin somewhat after the time of administration and continue for varying periods thereafter depending, e.g., on various factors such as the administration route, formulation, time required for absorption, distribution, cell uptake, etc. In some embodiments exposing a cell to an agent comprises contacting cells with a second agent, wherein the second agent induces the cell to express the first agent. For example, in some embodiments a cell that comprises an exogenous nucleic acid comprising an open reading frame operably linked to an inducible promoter is exposed to a protein encoded by the open reading frame by contacting the cell with an inducer that causes the cell to express the protein.

In some embodiments a method of enhancing the ability of an EMT-TF to induce a cell to become or remain a stem cell comprises: (a) providing a cell that comprises an EMT-TF; and (b) exposing the cell to an EMT-cooperating agent. In some embodiments a method of enhancing the ability of an EMT-TF to induce a cell to become or remain a stem cell comprises: (a) providing a cell that comprises an EMT-TF; and (b) increasing the amount or activity of at least one EMT-cooperating TF in the cell. In some embodiments the cell of step (a) ectopically expresses an EMT-TF. In some embodiments the cell of step (a) expresses an endogenous EMT-TF. In some embodiments the cell of step (a) has been or is being induced to undergo EMT.

In some embodiments a method of generating stem cells comprises steps of: (a) providing a population of cells that exhibit one or more epithelial characteristics and one or more mesenchymal characteristics; and (b) exposing the population of cells to an EMT-cooperating agent, thereby generating stem cells in the population. In some embodiments a method of generating stem cells comprises steps of: (a) providing a population of cells that exhibit one or more epithelial characteristics and one or more mesenchymal characteristics; and (b) increasing the amount or activity of at least one EMT-cooperating TF in the population of cells, thereby generating stem cells in the population. In some embodiments the cells express significant levels of an endogenous EMT-TF. In some embodiments a method comprises isolating cells that express significant levels of an endogenous EMT-TF from a population of cells that comprises cells that express significant levels an endogenous EMT-TF and cells that do not express significant levels of said endogenous EMT-TF and then exposing said isolated cells to an EMT-cooperating agent. In some embodiments “significant levels of an endogenous EMT-TF” refer to levels sufficient to cause the acquisition of one or more mesenchymal characteristics by a differentiated epithelial cell, that does not naturally exhibit said characteristic(s). In some embodiments a population of cells that exhibit one or more epithelial characteristics and one or more mesenchymal characteristics comprises epithelial cells that have been exposed to an EMT-inducing agent. In some embodiments a population of cells that exhibit one or more epithelial characteristics and one or more mesenchymal characteristics comprises progenitor cells.

In some aspects the disclosure provides methods of converting a cell to a less differentiated state (“de-differentiation”). In general, a de-differentiated cell has lost one or more of the specialized features found in the original differentiated cell, e.g., one or more features that distinguishes the differentiated cell from many or most other differentiated cell types and/or that confers or contributes to conferring on the differentiated cell an ability to perform a particular functional or structural role in the body. In some embodiments a method of converting a cell to a less differentiated state comprises: (a) providing a cell; and (b) increasing the amount or activity of at least one EMT-cooperating TF in the cell, thereby converting the cell to a less differentiated state. In some embodiments a method of converting a cell to a less differentiated state comprises: (a) providing a cell; and (b) contacting the cell with an EMT-cooperating agent, thereby converting the cell to a less differentiated state. In some embodiments a method of converting a cell to a less differentiated state comprises: (a) providing a cell; and (b) inducing EMT and increasing the amount or activity of at least one EMT-cooperating TF in the differentiated cell, thereby converting the cell to a less differentiated state. In some embodiments the cell is a progenitor cell. In some embodiments the cell is an epithelial cell. In some embodiments the cell is a partially differentiated cell.

In some embodiments of any aspect herein pertaining at least in part to an epithelial cell the cell is a differentiated epithelial cell. In some embodiments the cell is a differentiated luminal epithelial cell. In some embodiments the cell is a differentiated epithelial cell. In some embodiments the cell is a differentiated myoepithelial cell. In some embodiments the cell is a partially differentiated cell. In some embodiments a differentiated epithelial cell is terminally (fully) differentiated, e.g., it is the last cell in a lineage of cells and does not give rise to a more differentiated or more functionally specialized cell.

In some aspects the disclosure provides methods of expanding the differentiation potential of a cell. In some embodiments a method of expanding the differentiation potential of a cell comprises: (a) providing a cell; and (b) increasing the amount or activity of at least one EMT-cooperating TF in the differentiated cell, thereby expanding the differentiation potential of a cell. In some embodiments a method of expanding the differentiation potential of a cell comprises: (a) providing a cell; and (b) contacting the cell with an EMT-cooperating agent, thereby expanding the differentiation potential of a cell. In some embodiments a method of expanding the differentiation potential of a cell comprises: (a) providing a cell; and (b) inducing EMT and increasing the amount or activity of at least one EMT-cooperating TF in the differentiated cell, thereby expanding the differentiation potential of a cell. In some embodiments the cell is a progenitor cell. In some embodiments the cell is a differentiated epithelial cell. In some embodiments the cell is a differentiated luminal epithelial cell. In some embodiments the cell is a differentiated epithelial cell. In some embodiments the cell is a differentiated myoepithelial cell. In some embodiments a method of expanding the differentiation potential of a cell comprises: (a) providing a differentiated cell; and (b) contacting the cell with an EMT-cooperating agent, thereby enhancing the ability of the cell to dedifferentiate to a cell that has ability to differentiate into more distinct cell types than the original cell. In some embodiments a method of expanding the differentiation potential of a cell comprises: (a) providing a differentiated cell; and (b) increasing the amount or activity of at least one EMT-cooperating TF in the differentiated cell, thereby enhancing the ability of the cell to dedifferentiate to a cell that has ability to differentiate into more distinct cell types than the original cell. In some embodiments either of the foregoing methods comprises inducing EMT in the differentiated cell.

In some embodiments of any aspect pertaining at least in part to an EMT-cooperating TF, the EMT-cooperating TF comprises a Sox protein or a functional variant thereof. Sox proteins are transcription factors that contain a high mobility group (HMG) box that confers DNA binding ability to the protein. As described herein, Sox proteins can cooperate with EMT-inducing agents to promote the generation or maintenance of stem cells. In some embodiments, expression of a Sox protein in a population of epithelial cells in combination with expression of an EMT-TF generates substantially more stem cells than would result from either (i) expressing a Sox protein and not expressing an EMT-TF or (ii) expressing an EMT-TF and not expressing a Sox protein. For example, Applicants found that ectopically expressing the TF Sox9 in mammary epithelial cells (MECs) concomitantly with ectopic expression of the EMT-TF Slug dramatically increased the number of mammary stem cells (MaSCs) formed, as compared with expressing Slug alone. Mammary stem cells (MaSCs) are a subset of mammary epithelial cells that reside in the mammary gland and are capable, following appropriate manipulation, of spawning an entire normal mammary gland, this being usually measured following experimental implantation in an appropriate location in a mouse host. The normal mouse and human mammary glands are composed of a number of distinct cell types that derive ultimately from MaSCs. Among these are luminal cells and basal cells and within each of these two compartments there are more differentiated and less differentiated subtypes, the latter being termed, in the present application, progenitors or progenitor cells. Applicants also found that ectopic expression of Sox9 in a population of epithelial cells that already expressed endogenous Slug markedly increased the number of stem cells. In addition, ectopic expression of Sox9 converted differentiated luminal MECs into luminal progenitor cells, which could be converted into stem cells by inducing EMT. In some embodiments, maintaining expression of Sox protein in a population of cells comprising stem cells in combination with maintaining expression of an EMT-TF results in maintaining a markedly greater number of stem cells than would result if expression of either the Sox protein or the EMT-TF were inhibited. For example, Applicants found that knockdown of either Slug or Sox9 by RNA interference (RNAi) in a population of MECs greatly reduced the number of stem cells while having only modest effects on overall cell number over the same time period. Applicants further found that coexpression of Slug and Sox9 promotes the tumorigenic and metastasis-seeding abilities of human breast cancer cells and is associated with poor patient survival, providing direct evidence that human breast cancer stem cells are also controlled by these regulators.

In some embodiments expression of an EMT-TF or EMT-cooperating TF induces expression of its endogenous counterpart and/or induces expression of one or more other TFs that function in the same process or pathway. For example, Applicants found that expression of exogenous Slug and Sox9 in epithelial cells led to the induction of endogenously expressed EMT-TFs, including Twist2, Zeb1, and Slug itself, as well as endogenously expressed Sox factors, including Sox9 and its close paralog Sox10. Hence, the ectopically expressed Slug and Sox9 induced expression of their corresponding endogenous counterparts or paralogs, forming a self-reinforcing auto-regulatory network that contributed to maintenance of the SC program. The resulting cells retained stem cell properties for some time even after expression of the exogenous proteins was turned off by withdrawal of the agent that was used to induce expression of exogenous Slug and Sox9. Differentiated cells arising from these cells turned off expression of the endogenous Slug and Sox9 demonstrating that the induced EMT was reversible.

In some embodiments, methods disclosed herein comprise transiently expressing an exogenous EMT-TF or EMT-cooperating protein (e.g., an EMT-cooperating TF) in an epithelial cell. In some embodiments transient expression is achieved without modifying the genomic DNA sequence of the cell or an ancestor of the cell. In some embodiments transient expression is achieved by means that do not require introducing an exogenous nucleic acid into the cell or an ancestor of the cell. In some embodiments transient expression results in detectably increased levels of the transiently expressed (e.g., ectopically expressed) protein for between 12 hours and 60 days, e.g., between 1-5 and 30 days, e.g., or any subrange thereof. In some embodiments transient expression results in an increase by at least a factor of 2, 5, 10, 20, 50, or 100-fold or more relative to levels existing prior to the manipulation that resulted in transient expression. In some embodiments transient expression is robust, readily detectable expression. In some embodiments transient expression is at a level that places the transiently expressed gene among the 50%, 40%, 30%, 20%, or 10% of genes most highly expressed by the cell during at least part of the period of transient expression. In some embodiments, methods disclosed herein comprise expressing an exogenous EMT-TF for a sufficient period of time to induce expression of at least one endogenous EMT-TF. In some embodiments, methods disclosed herein comprise expressing an exogenous EMT-cooperating TF in an epithelial cell for a sufficient period of time to induce expression of at least one endogenous EMT-cooperating TF. In some embodiments, methods disclosed herein comprise expressing an exogenous EMT-TF and an exogenous EMT-cooperating TF in an epithelial cell for a sufficient period of time to induce expression of at least one endogenous EMT-TF and at least one endogenous EMT-cooperating TF. In some embodiments a sufficient time is at least about 5 days, e.g., at least about 5-10 days. In some embodiments a longer time period, e.g., about 10-20 days, or 20-30 days, is used. In some embodiments, methods disclosed herein comprise introducing a polypeptide comprising an EMT-TF or EMT-cooperating TF into an epithelial cell. In some embodiments a stem cell state resulting from transient expression of an exogenous EMT-TF and an exogenous EMT-cooperating TF lasts for at least about 5 days, e.g., at least about 5-10 days, about 10-30 days, 30-60 days, or more, e.g., months, years, or indefinitely. In some embodiments the expression by a cell of an endogenous EMT-TF and/or endogenous EMT-cooperating TF resulting from transient expression of the exogenous EMT-TF and/or EMT-cooperating TF is reversible, e.g., in daughters arising from the cell.

In some embodiments a method comprises ectopically expressing a polypeptide comprising an EMT-TF or EMT-cooperating TF or other protein of interest in a cell by introducing into the cell a nucleic acid that encodes a polypeptide comprising the protein of interest. For example, in some embodiments stem cells are generated by introducing into a population of epithelial cells a first nucleic acid that encodes a polypeptide comprising an EMT-TF and a second nucleic acid that encodes a polypeptide comprising an EMT-cooperating TF and maintaining the cells under conditions in which the polypeptides are produced. In some aspects, such nucleic acids, vectors comprising them, and cells comprising the nucleic acids or vectors are provided. In some embodiments the first and second nucleic acids are portions of a single, larger nucleic acid. In some embodiments the first and second nucleic acids are separated nucleic acids that are not part of a larger nucleic acid. In some embodiments the nucleic acid comprises a cDNA encoding the polypeptide or comprises a continuous open reading frame encoding the polypeptide that does not require splicing. In some embodiments the nucleic acids are introduced using a single vector. In some embodiments the nucleic acids are introduced using different vectors.

In various embodiments a “protein of interest” can be any protein. In some embodiments a protein of interest is an EMT-inducing agent, e.g., an EMT-TF, such as Slug. In some embodiments a protein of interest is an EMT-inducing agent other than an EMT-TF. In some embodiments a protein of interest is an EMT-cooperating agent, e.g., an EMT-cooperating TF, such as Sox9 or Sox10. In some embodiments a protein of interest is an EMT-cooperating agent other than an EMT-TF. In some embodiments a protein of interest is one for which the cell is to be used as a source ex vivo or in vivo. For example, in some embodiments a cell is engineered to produce a protein that is lacking in a subject (e.g., insulin in the case of a subject with Type I diabetes). The cells may be used as a source of the protein ex vivo or may be introduced into the subject, where they serve as an in vivo source of the protein. In some embodiments a protein of interest comprises a reporter molecule, e.g., a detectable polypeptide, allowing the cells to be readily detected and/or isolated and/or allowing monitoring of excision of integrated DNA or loss of non-integrated nucleic acid (e.g., as discussed further below). Exemplary reporter molecules include, e.g., green, blue, sapphire, yellow, red, orange, and cyan fluorescent proteins and derivatives thereof; monomeric red fluorescent protein and derivatives such as those known as “mFruits”, e.g., mCherry, mStrawberry, mTomato; enzymes such as luciferase; beta-galactosidase; horseradish peroxidase; alkaline phosphatase, etc. In some embodiments a protein of interest comprises a protein with an extracellular domain, which may be used to isolate or target an agent to a cell that expresses it.

In some embodiments a RNA or protein of interest comprises a naturally occurring sequence. In some embodiments a RNA or protein of interest comprises a variant of a naturally occurring sequence. For example, in some embodiments an engineered variant has at least one altered property. For example, in some embodiments an engineered variant has altered (e.g., higher or lower) activity or stability or responsiveness to a ligand.

In some embodiments a polypeptide comprises a domain that renders the polypeptide responsive to a ligand. For example, a polypeptide in some embodiments comprises at least a portion of a ligand-binding domain of a hormone receptor, such as at least a portion of the estrogen receptor (ER) ligand-binding domain (LBD) or an altered version thereof. Fusion with the ligand binding domain renders the activity of the protein dependent on the presence of an ER ligand, such as a naturally occurring ER ligand (e.g., 17,B-estradiol) or synthetic ER ligand (e.g., tamoxifen). In some embodiments an altered LBD of the human or mouse ER (Gly 521->Arg) is used, resulting in a chimeric protein that does not bind17,B-estradiol, whereas it binds the synthetic ligands tamoxifen and 4-hydroxytamoxifen (OHT). For example, in some embodiments an EMT-inducing agent comprises a polypeptide comprising an EMT-TF and a domain that renders the activity of the EMT-TF dependent on a ligand, e.g., an ER ligand. In some embodiments an EMT-cooperating agent comprises a polypeptide comprising an EMT-cooperating TF and a domain that renders the activity of the EMT-cooperating TF dependent on a ligand, e.g., an ER ligand. In some embodiments activity of the protein is regulated by the ligand. For example, activity is induced by adding the ligand to culture medium containing the cells or administering the ligand to a subject to whom the cells have been introduced. Activity is inhibited by ending exposure to the ligand, e.g., by changing the culture medium or simply or ceasing to administer the ligand. In some embodiments withdrawal of an inducer promotes differentiation of the stem or progenitor cells or daughter cells derived therefrom. It will be understood that the ER LBD system is exemplary of various inducible systems. A variety of different proteins are cytoplasmic (e.g., via binding to heat shock protein 90 complex) but released therefrom upon ligand binding. Ligand binding domains of such proteins can be used to render activity of a protein of interest responsive to the ligand. In some embodiments the protein of interest comprises or is modified to comprise a targeting sequence, e.g., a nuclear targeting sequence. In some embodiments 2, 3, 4, or more proteins of interest are ectopically expressed. One or more of the proteins may be ligand-responsive. In some embodiments two or more proteins can be independently regulated, e.g., they are responsive to different ligands. In some embodiments two or more proteins are regulated using the same inducer.

In some embodiments two or more proteins are encoded by different mRNAs. In some embodiments two or more proteins are encoded by a single RNA, e.g., comprising a single open reading frame. For example, an internal ribosome entry site (IRES) or a self-cleaving peptide, e.g., a 2A peptide, can be used to produce multiple proteins from a single mRNA. The self-cleaving 18-22 amino acids long 2A peptides mediate ‘ribosomal skipping’ between the proline and glycine residues and inhibit peptide bond formation without affecting downstream translation. These peptides allow multiple proteins to be encoded as polyproteins, which dissociate into component proteins upon translation. Use of the term “self-cleaving” is not intended to imply proteolytic cleavage reaction. Self-cleaving peptides are found in members of the Picornaviridae virus family, including aphthoviruses such as foot-and-mouth disease virus (FMDV), equine rhinitis A virus (ERAV), Thosea asigna virus (TaV) and porcine tescho virus-1 (PTV-I) (Donnelly, M L, et al, J. Gen. Virol, 82, 1027-101 (2001); Ryan, M D, et al., J. Gen. Virol., 72, 2727-2732 (2001) and cardioviruses such as Theilovirus (e.g., Theiler's murine encephalomyelitis) and encephalomyocarditis viruses. Aphthovirus 2A polypeptides are typically ˜18-22 amino acids long and contain a Dx1Ex2NPG, where x1 is often valine or isoleucine. As noted above, the 2A sequence is believed to mediate ‘ribosomal skipping’ between the proline and glycine, impairing normal peptide bond formation between the P and G without affecting downstream translation. An exemplary 2A sequence is VKQTLNFDLLKLAGDVESNPGP (SEQ ID NO. 61). In some embodiments a polycistronic vector comprising a portion that encodes a polypeptide comprising an EMT-inducing TF and a portion that encodes a polypeptide comprising an EMT-cooperating TF is used to ectopically express the TFs in an epithelial cell. In some embodiments a polycistronic vector encodes 2, 3, 4, or more distinct proteins, wherein the coding sequences for such proteins are separated by 2A sequences. In some embodiments at least one protein comprises an EMT-TF and at least one protein comprises an EMT-cooperating TF.

In some embodiments a method comprises ectopically expressing a RNA of interest in a cell by introducing into the cell a nucleic acid that encodes the RNA (e.g., DNA that serves as a template for transcription that results in synthesis of the RNA). In various embodiments a “RNA of interest” can be any RNA. In some embodiments the RNA does not encode a protein. In some embodiments an RNA comprises a miRNA precursor, short hairpin RNA, tRNA, miRNA sponge, antisense RNA, or ribozyme. In some embodiments an RNA encodes a protein, e.g., following transcription (and, in some embodiments, processing) the RNA or a portion thereof is translated to a protein of interest.

In some embodiments a nucleic acid comprises appropriate expression control elements (e.g., a promoter), operably linked to a sequence coding for an RNA of interest or a polypeptide comprising a protein of interest, such that the nucleic acid is transcribed and the encoded RNA or polypeptide is produced by the cell. Nucleic acids can be produced using standard methods known in the art, such as recombinant nucleic acid technology, amplification (e.g., using PCR), chemical synthesis, or combinations thereof. See, e.g. Sambrook, supra, and Ausubel, supra. Nucleic acid sequences, e.g., nucleic acids encoding a protein of interest (e.g., a TF) can be isolated from cells that contain such sequences or obtained from other sources (e.g., libraries or vectors that already contain previously isolated sequences) and manipulated using methods known in the art. In some embodiments a nucleic acid is inserted into a vector such as a plasmid or virus. In some embodiments a nucleic acid is introduced into cell using a vector such as a plasmid or virus into which the nucleic acid has been inserted. Various techniques can be employed for introducing nucleic acid molecules into cells as known in the art. Exemplary techniques include transfection (e.g., calcium-phosphate-mediated transfection), electroporation, infection with a virus that contains the nucleic acid, particle bombardment, microinjection, magnetofection, etc. Transfection may be facilitated by use of a suitable transfection reagent. Numerous lipid-based or non-lipid based transfection reagents are known. Examples of commercially available transfection reagents include, e.g., Lipofectamine, Effectene, Polyfect, Geneporter, HiPerfect, and numerous others. One of ordinary skill in the art will be aware of transfection reagents that are suitable or optimized for introducing various types of nucleic acids (e.g., plasmids, oligonucleotides, RNA (e.g., siRNA, mRNA) into cells of various types. In some embodiments a nucleic acid is introduced into cells in culture. In some embodiments transfection is repeated, e.g., the population of cells is transfected multiple times, e.g., up to about 10-20 times, e.g., daily or every other day, for example. In some embodiments cells are subjected to selection (e.g., drug selection), e.g., to eliminate cells that have not been successfully transfected.

In some embodiments genetic modification is used to achieve expression of an EMT-TF and EMT-cooperating TF or other protein of interest. For example, in some embodiments a nucleic acid that encodes a polypeptide comprising a protein of interest is integrated into the genome of the cell. In some embodiments integration is targeted to a predetermined locus in the cell, e.g., via homologous recombination. In some embodiments an endonuclease that is targeted to selected DNA sequences so as to cause chromosomal double-stranded DNA breaks (DSBs), which stimulate breakage repair mechanisms such as non-homologous end-joining or homologous recombination is used. Proteins that comprise a DNA binding domain (DBD) capable of recognizing a selected target DNA sequence and a cleavage domain (e.g., a cleavage domain of a non-specific endonuclease such as FokI or a variant thereof) may be used. In some embodiments a zinc finger nuclease, TAL effector nuclease (TALEN), or meganuclease is used to direct integration to a predetermined locus. In some embodiments a predetermined locus is a safeharbor locus such as the Col1A1 or PPP1R12C gene (also termed the AAVS1locus). A safe harbor locus is one whose disruption, e.g., in one or both chromosomal copies, does not adversely affect a cell or descendants of the cell or, in some embodiments, does not result in a phenotypic change in the cell or descendants of the cell. In some embodiments a safe harbor locus is one whose disruption, e.g., in one or both chromosomal copies, does not adversely affect a tissue, organ, or an organism derived at least in part from the cell or, in some embodiments, does not result in a phenotypic change such tissue, organ, or organism. In some embodiments integration is not targeted to a predetermined locus. Genetic modifications of interest in various embodiments may include gene disruption (e.g., by targeted insertions or deletions), introduction of discrete base substitutions specified by a homologous donor DNA), or targeted insertion into a selected native genomic locus of DNA whose expression is desired (e.g., inserting a promoter or altering a nonfunctional promoter to a functional promoter). In some embodiments such modifications may be performed without using a selectable marker.

As noted above, in some embodiments transient expression is used to achieve ectopic expression of an EMT-inducing agent and an EMT-cooperating agent. Transient expression may employ any of various strategies in various embodiments. In some embodiments regulatable, e.g., inducible, expression control elements are used. Regulatable expression systems, e.g., tetracycline-regulatable promoters, are known in the art (see Glossary). In some embodiments inducible or repressible expression is used to achieve transient ectopic expression. In some embodiments a fusion protein comprising a domain such as a hormone receptor LBD is used to achieve transient expression (see, e.g., discussion above).

In some embodiments, a method of generating stem cells does not involve genetic modification, e.g., insertion of exogenous genetic material into the genome. Without limiting the disclosure in any way, it is noted that avoiding genetic modification may be desirable, e.g., when generating cells that will be used for cell-based therapy. In some embodiments expression of a polypeptide comprising an EMT-inducing TF or comprising an EMT-cooperating TF is achieved by introducing RNA encoding the polypeptide into a cell, wherein the RNA does not give rise to DNA that integrates into the genome of the cell. In some embodiments the RNA is mRNA, which may have been isolated from another cell. In some embodiments the RNA comprises one or more modifications that, for example, increase its stability and/or reduce an immune response that may otherwise be directed thereto. See, e.g., Warren, L., et al., Volume 7(5): 618-630, 2010, for exemplary modifications that may be used in some embodiments.

In some embodiments expression, e.g., transient expression, of a polypeptide comprising an EMT-inducing TF or comprising an EMT-cooperating TF or other protein of interest is achieved by introducing a plasmid, e.g., a DNA plasmid, encoding the polypeptide into a cell. In some embodiments transient expression of a polypeptide comprising an EMT-inducing TF or comprising an EMT-cooperating TF is achieved by introducing a non-integrating episomal vector into a cell, wherein the non-integrating vector comprises a sequence encoding the polypeptide, operably linked to expression control elements sufficient to direct expression. A variety of extrachromosomal elements known in the art may be used in various embodiments (see, e.g., Wade-Martins R. Developing extrachromosomal gene expression vector technologies: an overview. Methods Mol Biol. 738:1-17, 2011). In some embodiments a non-integrating episomal vector is an oriP/EBNA1 (Epstein-Barr nuclear antigen-1)-based episomal vector. The stable extrachromosomal replication of oriP/EBNA1 vectors in mammalian cells requires only a cis-acting oriP element and a trans-acting EBNA1 gene. The oriP/EBNA1 vectors replicate typically only once per cell cycle, and with drug selection can be established as stable episomes in a small percentage (e.g., about 1%) of the initial transfected cells. If drug selection is subsequently removed, the episomes are lost at an appreciable frequency, e.g., about 5% per cell generation, due to defects in plasmid synthesis and partitioning. Therefore, cells devoid of plasmids can be isolated readily. In some embodiments resulting cells are free of vector and transgene sequences. In some embodiments expression, e.g., transient expression, comprising an EMT-inducing TF or comprising an EMT-cooperating TF or other protein of interest is achieved using an excisable expression cassette. In some embodiments, a nucleic acid that has integrated into the genome is (after transient expression therefrom) at least in part excised from the genome, e.g., by site-specific recombination (which term refers to the enzyme-mediated cleavage and ligation of two defined polynucleotide sequences), and the resulting break repaired. Site-specific recombinase systems include, e.g., the Lox/Cre, Flp/Frt systems. In some embodiments the nucleic acid comprises at least one site for a recombinase, such that following insertion at least a portion of the integrated nucleic acid is flanked by sites for a recombinase (e.g., LoxP sites). Introduction of the recombinase into the cell results in excision of the flanked region and, in some embodiments, at least a portion of the recombinase site(s). In various embodiments the recombinase can be introduced by any of variety of ways. In some embodiments the recominase is transiently expressed, e.g., a RNA or a non-integrating vector encoding the recombinase is introduced into the cell. In some embodiments a piggy Bac transposon system is used to achieve transient ectopic expression without a permanent gentic modification. In some embodiments an adenovirus vector system is used to achieve transient ectopic expression without permanent genetic modification. In some embodiments protein transduction can be used to achieve transient presence of a TF in a cell. If desired, the absence or identity of exogenously introduced nucleic acids can be verified or determined, e.g., using a variety of methods such as Southern blotting, PCR amplification with appropriate primers, Northern blot, sequencing, etc.

In some aspects, the disclosure encompasses the recognition that stem cells having multi-lineage potential may be generated from differentiated epithelial cells by activating, in such differentiated epithelial cells, two or more gene expression programs characteristic of different cell lineage programs. As will be understood by one of ordinary skill in the art, a gene expression program typically comprises expression of multiple genes (e.g., activating the expression of multiple genes) and in at least some instances may include repression of one or more genes. Whether or not a gene expression program is activated in a cell is (or could be) determined by any of a variety of methods. In some embodiments, assessing whether or not a gene expression program is activated in a cell comprises obtaining a gene expression profile. In some embodiments, assessing whether or not a gene expression program is activated in a cell comprises assessing expression of one or more “signature genes”. Signature genes may be identified using any of a variety of approaches known in the art. In some embodiments signature genes are identified by first sorting cells from a particular tissue or organ into distinct subsets or subpopulations based on, e.g., morphology, location, expression of cell surface markers, functional properties, etc., and then identifying genes that are characteristic of each subset. Such genes may, for example, be overexpressed or underexpressed in a particular subset or subpopulation as compared with their average expression level across all subsets or subpopulations. Signature genes may be identified or their expression measured using various methods known in the art for gene expression measurement, e.g., methods useful for measuring RNA or protein. In general, methods useful for measuring RNA include, e.g., microarray hybridization (e.g., using cDNA or oligonucleotide microarrays), reverse transcription PCR (e.g., real-time reverse transcription PCR; quantitative RT-PCR), reverse transcription followed by sequencing, nanostring technology (Geiss, G., et al., Nature Biotechnology (2008), 26, 317-325), flow cytometry, in situ hybridization (e.g., fluorescence in situ hybridization), Northern blots, etc. The TaqMan® assay and the SYBR® Green PCR assay are commonly used real-time PCR techniques. Other assays include the Standardized (Sta) RT-PCR™ (Gene Express, Inc., Toledo, Ohio) and QuantiGene® (Panomics, Inc., Fremont, Calif.), etc. Methods useful for measuring protein include, e.g., immunologically based methods such as enzyme-linked immunosorbent assay (ELISA), bead-based assays such as the Luminex® assay platform (Invitrogen/Life Technologies), protein microarrays, surface plasmon resonance assays (e.g., using BiaCore® technology), immunoprecipitation, Western blot, flow cytometry. As used herein, the term “ELISA” encompasses assays that involve use of primary or secondary antibodies linked to an enzyme, which acts on a substrate to produce a detectable signal (e.g., production of a colored product) to indicate the presence of antigen or other analyte and use of non-enzymatic reporters such as fluorogenic, electrochemiluminescent, or real-time PCR reporters that generate quantifiable signals and includes variations such as “indirect”, “sandwich”, “competitive”, and “reverse” ELISA. One of ordinary skill in the art will be able to select an appropriate measurement method for a particular application. In some embodiments a method that allows the measurement of large numbers, e.g., hundreds or thousands, of gene expression products in parallel, such as microarray analysis or RNA-Seq is used to initially identify a gene signature set. Gene expression measurements can be analyzed using a variety of methods known in the art such as cluster analysis (e.g., hierarchical clustering), e.g., to determine a gene expression profile or gene signature set characteristic of a particular cell type or differentiation state. In some embodiments RT-PCR or flow cytometry is used to validate a signature gene set or to subsequently determine whether a cell or population of cells expresses one or more genes in the gene signature set. In some embodiments a signature gene set comprises at least 2, 3, 4, 5, 6, 7, 8, 9, or 10 genes. In some embodiments a signature gene set is known in the art. For example, exemplary signature genes of various mammary epithelial cell subpopulations in both human and mouse have been identified (see, e.g., Lim et al., 2010). One of ordinary skill in the art will understand that signature gene sets can be selected in a variety of ways, and often any of various different signature gene sets could reasonably be used for a particular cell lineage or subpopulation. In some embodiments methods of assessing whether or not a gene expression program is activated include, e.g., measuring promoter occupancy using, e.g., chromatin immunoprecipitation (e.g., ChIP-on-Chip or ChIP-Seq), assessing DNA or histone modifications, etc. In some embodiments a cell comprises a reporter gene, and assessing whether a gene expression program is activated comprises detecting an expression product of the reporter gene. In some embodiments the reporter gene comprises one or more expression control elements, e.g., a promoter, found in a signature gene, so that expression of the reporter gene serves as a surrogate for expression of the signature gene.

In some embodiments a method of generating a stem cell having multi-lineage potential from a differentiated epithelial cell comprises activating at least two gene expression programs in the differentiated epithelial cell, wherein a first gene expression program is characteristic of a first lineage and a second gene expression program is characteristic of a second lineage, thereby producing a stem cell capable of generating daughters that can enter the first lineage program (and give rise to cells of the first lineage) and daughters that can enter the second lineage program (and give rise to cells of the second lineage). In some embodiments a bipotential stem cell is generated. In some embodiments activating a gene expression program comprises expressing a TF in the cell, wherein the TF regulates expression of at least some of the genes of the gene expression program. In some embodiments the TF is a Sox protein. In some embodiments the TF is an EMT-TF. In some embodiments a first gene expression program is activated by an EMT-TF and a second gene expression program is activated by a TF that cooperates with the EMT-TF, e.g., a Sox protein. In some embodiments at least two cell lineages are epithelial lineages. In some embodiments a cell lineage is a luminal cell lineage. In some embodiments a cell lineage is a myoepithelial cell lineage. In some embodiments the first and second cell lineages generate cells found in a particular organ or tissue of interest, such as the breast, intestine, liver, pancreas, or skin. One of ordinary skill in the art will understand that the cell lineages and corresponding gene expression programs will differ depending on the particular organ or tissue of interest. In some embodiments a TF that activates a gene expression program of a cell lineage of interest is identified as described herein for Sox9. In some embodiments a gene expression program for a particular cell lineage pathway is activated by an EMT-TF. In some embodiments an appropriate EMT-TF for activating a gene expression program for a particular cell lineage pathway is identified as described herein for Slug.

In some embodiments SCs generated as described herein will give rise ex vivo to at least some, most, or all of the characteristic epithelial cell types found in the organ or tissue from which the cells used to generate such SCs were obtained. In some embodiments SCs generated as described herein, when introduced into a subject into an organ or tissue corresponding to the location from which the cells used to generate such SCs were obtained, will give rise to at least some, most, or all of the characteristic epithelial cell types of which the organ or tissue is composed. In some embodiments, SCs generated as described herein, when introduced into a subject into an organ or tissue corresponding to the location from which the cells used to generate such SCs were obtained, will give rise in vivo to a functional organ or tissue and/or integrate appropriately into an existing organ or tissue.

In some embodiments an EMT-TF that functions in SCs in a tissue or organ of interest and/or a TF that cooperates with that EMT-TF in generation of such SCs are identified. In some embodiments, the EMT-TF is identified by a method comprising: (a) obtaining a mixed population of cells (i.e., a population that has not been subjected to sorting or other means of separating cells into distinct subpopulations) from the tissue or organ; (b) assessing expression of multiple EMT-TFs therein; and (c) identifying an EMT-TF that is expressed at a significantly higher level than most or all other EMT-TFs assessed. In some embodiments, the EMT-TF is identified by obtaining a subpopulation from the tissue or organ, wherein the subpopulation is enriched for stem cells and/or progenitor cells, assessing expression of multiple EMT-TFs therein, and identifying an EMT-TF that is expressed at significantly higher levels than most or all other EMT-TFs assessed in the subpopulation. In some embodiments a TF that cooperates with the EMT-TF in generating SCs is identified by a method comprising: (a) assessing expression of multiple different TFs in cells that express the EMT-TF (or in some embodiments in a mixed cell population); and (b) identifying a TF that is expressed at a significantly higher level than most or all other TFs assessed. In various embodiments the number of EMT-TFs whose expression is assessed is at least 5. In various embodiments the number of TFs whose expression is assessed to identify a TF that cooperates with the EMT-TF is at least 5. In some embodiments TFs to be assessed are selected from among TFs that are known to be expressed in stem or progenitor cells and/or in developmental processes.

In some embodiments, a method of identifying a cell that has multi-lineage potential comprises steps of: (a) providing a sample comprising at least one cell; and (b) assessing expression of a first gene that encodes or is regulated by an EMT-TF and a second gene that encodes or is regulated by an EMT-cooperating TF in at least one cell of the sample; and (c) identifying a cell that has increased expression of the first and second genes, thereby identifying a cell that has multi-lineage potential. In some embodiments the sample comprises multiple cells, and the method further comprises separating cells that have increased expression of the first and second genes from cells that do not have increased expression of both of the genes.

The present disclosure provides a variety of agents, compositions, and methods. In some embodiments, a method is performed in vitro (i.e., outside the body of an organism, e.g., in a cell culture vessel). In some embodiments, a method is performed in vivo, e.g., by administering one or more agents or compositions to a subject. In some embodiments, a method is performed at least in part in vitro, e.g., cells are contacted with an agent or composition in vitro, and cells are subsequently introduced into a subject, e.g., for experimental or therapeutic purposes. Thus it should be understood that unless otherwise indicated or otherwise evident from the context, any method described herein can encompass in vitro and in vivo embodiments, and any agent or composition can be employed in vitro or in vivo in various embodiments. In various embodiments, all different combinations of epithelial cell, EMT-inducing agent, and EMT-cooperating agent are provided. In various embodiments, all different combinations of epithelial cell, method of inducing EMT, and method of increasing expression or activity of an EMT-cooperating TF are provided. In various embodiments, all different combinations of epithelial cell, EMT-TF, and EMT-cooperating TF are provided. In some embodiments of any aspect herein pertaining at least in part to an EMT-TF, the EMT-TF is Slug, Snail, Twist1, Twist2, Zeb1, Zeb2, Goosecoid, FoxC2, Tcf3, Klf8, FoxC1, FoxQ1, Six1, Lbx1, Yap1, and HIF-1 or a functional variant thereof. For example, in some embodiments of any aspect herein pertaining at least in part to an EMT-TF, the EMT-TF is Slug or Snail or a functional variant of either. In some embodiments of any aspect herein pertaining to an EMT-cooperating TF, the EMT-cooperating TF is a Sox protein, e.g., a SoxE protein, e.g., Sox9 or Sox10, or a functional variant. In some embodiments of any aspect herein pertaining at least in part to an EMT-TF and an EMT-cooperating TF, the EMT-TF and the EMT-cooperating TF are selected such that they are capable of cooperating with each other to, e.g., promote generation of stem cells from epithelial cells. In some embodiments of any aspect herein pertaining at least in part to cell(s), the cell(s) are human cell(s). In some embodiments of any aspect herein pertaining at least in part to a subject, the subject is human. In some embodiments of any aspect herein pertaining at least in part to a protein, e.g., an EMT-TF or an EMT-cooperating protein such as an EMT-cooperating TF, the protein comprises a human protein, or, in some embodiments, a functional variant thereof. It should be understood that where a method, product (e.g., a cell), or composition described herein relates or pertains at least in part to an organism of a particular species, certain embodiments comprise use of EMT-cooperating agents (e.g., EMT-cooperating TFs) and EMT-TFs corresponding to (e.g., native to) that species. For example, in some embodiments a method of generating a stem cell from a human epithelial cell, comprises ectopically expressing a protein comprising a human EMT-TF and a protein comprising a human EMT-cooperating TF in the human epithelial cell. One of ordinary skill in the art will appreciate that due to the degeneracy of the genetic code, a particular protein, e.g., a human protein, can be encoded by a human nucleic acid sequence or by any of a variety of other sequences encoding the same amino acids. One of ordinary skill in the art will also appreciate that proteins native to a particular mammalian species often can substitute for corresponding proteins native to other mammalian species with respect to one or more activities, particularly where high degrees of sequence identity exist, and such embodiments are encompassed herein.

In certain embodiments of any aspect herein, a culture medium or composition comprises a ROCK inhibitor. For example, in some embodiments an epithelial cell is cultured in culture medium comprising a ROCK inhibitor. In some embodiments a method comprises inducing EMT in an epithelial cell cultured in medium comprising a ROCK inhibitor. In some embodiments a method comprises ectopically expressing an EMT-cooperating TF in an epithelial cell cultured in medium comprising a ROCK inhibitor.

The concentration at which agents are used e.g., the concentration at which such agents are present in cell culture medium following addition thereto, can vary. The particular concentration will depend on the potency and identity of the agent, other agents used in combination therewith, and the desired result. Some non-limiting concentrations for certain agents are provided in the Examples. Exemplary, non-limiting ranges may vary between 0.1-fold and 10-fold from such concentrations, e.g., between 0.2-fold and 5-fold, or between 0.5-fold and 2-fold, in various embodiments.

In some aspects, conversion of differentiated cells to SCs by transient exogenous Slug and Sox9 expression, as described herein, demonstrates the existence of significant plasticity in the epithelial cell hierarchy. Without wishing to be bound by any theory, a metastable relationship may exist between SCs and differentiated cells, wherein certain tissue or tumor microenvironmental signals (e.g., endogenous secreted signaling molecules or cell-cell interactions) may be able to induce, e.g., transiently induce, the expression of one or more EMT-TFs (e.g., Slug) and one or more EMT-cooperating TFs (e.g., Sox9) therefore allowing de novo formation of SCs. In some embodiments, administration of agents that mimic or provide such signals (e.g., agonists of receptors through which such signals act) are useful to promote development of SCs, e.g., in noncancerous tissues in need of repair or regeneration. In some embodiments, antagonists of such signals or inhibitors of the relevant TFs are useful to inhibit development or persistence of CSCs, e.g., for treatment of cancer. In some embodiments, the disclosure encompasses methods comprising identifying such endogenous signals.

In some aspects, methods of preparing stem or progenitor cells are provided, the methods comprising inducing epithelial cells to undergo EMT and exposing the cells to an EMT-cooperating agent. In some embodiments any method of preparing stem or progenitor cells can further comprise separating cells that exhibit one or more stem or progenitor cell properties from cells that do not exhibit the particular propert(ies). In some embodiments separation is performed based on assessing expression of one or more markers.

In some aspects, cells prepared as described herein, and compositions comprising such cells, are provided. In various embodiments such cells and/or compositions have a variety of uses. Exemplary uses include cell-based therapies in which stem or progenitor cells derived from normal epithelial cells, or differentiated cells derived from such stem or progenitor cells, are transplanted or implanted into a subject (e.g., as described further below), methods for evaluating or screening biological activity of a therapeutic or biologically-active molecule in stem or progenitor cells, methods for identifying new and/or improved procedures and compounds for use in growing, maintaining and/or differentiating stem or progenitor cells, and/or for production including manufacturing of stem or progenitor cell-derived products such as endogenous proteins, recombinant proteins, peptides, fusion polypeptides, etc. Methods for evaluating or screening biological activities of therapeutic or biologically-active molecules such as screening to identify new lead compounds, and methods of identifying agents and conditions that favor the differentiation of stem or progenitor cells into particular cell lineages, are examples of other uses of progenitor cells. See, e.g., PCT/US2006/025589 (WO/2007/005611) for non-limiting discussion regarding stem/progenitor cells and uses thereof.

One of skill in the art will readily be able to obtain sequences of proteins disclosed herein, e.g., EMT-TFs and other EMT-inducing proteins, EMT-cooperating TFs, markers, etc., and the genomic and mRNA sequences encoding them, from publicly available databases, such as those available at the National Center for Biotechnology Information (NCBI; www.ncbi.nlm.nih.gov) or Universal Protein Resource (www.uniprot.org). Exemplary databases include, e.g., GenBank, RefSeq, Gene, UniProtKB/SwissProt, UniProtKB/Trembl, and the like. For example, the Gene database provides sequence and functional information, which can be obtained, e.g., by searching on a name or Gene ID for a gene or protein of interest. Table 1 provides gene names and Gene IDs for certain human genes of interest herein. One of ordinary skill in the art will readily be able to obtain the Gene IDs of corresponding genes in other organisms of interest. In general, sequences, e.g., mRNA and polypeptide sequences, in the NCBI Reference Sequence database may be used as gene product sequences for a gene of interest. In general, where aspects of this disclosure pertain to a gene or gene product, embodiments pertaining to allelic variants or isoforms are encompassed unless indicated otherwise. Certain embodiments may be directed to particular sequence(s), e.g., particular allele(s) or isoform(s). It is noted that the names of proteins and genes herein, whether written in upper case, lower case, or a combination of upper and lower case, italics, or non-italics, are intended to refer to the versions of such proteins and genes as found in any species of interest, e.g., any mammalian species, e.g., human, non-human primate, rodent, etc., unless otherwise specified or clearly evident from the context. For example, “Slug” refers to the human, non-human primate, rodent, etc., form of the gene and/or protein, as appropriate.

TABLE 1

| | Gene Official | | |
|---|---|---|---|
| Gene | Symbol | Gene ID (human) | RefSeq mRNA and Protein Acc. Nos. |
| Slug (also known | SNAI2 | 6591 | NM_003068.4 → NP_003059.1 |
| as Snail2) | | | |
| Snail | SNAI1 | 6615 | NM_005985.3 → NP_005976.2 |
| Twist1 | TWIST1 | 7291 | NM_000474.3 → NP_000465.1 |
| Twist2 | TWIST2 | 117581 | NM_057179.2 → NP_476527.1 |
| Zeb1 | ZEB1 | 6935 | NM_0011281282 → NP_001121600.1 |
| | | | (isoform a) |
| | | | NM_001174093.1 → NP_001167564.1 |
| | | | (isoform c) |
| | | | NM_001174094.1 → NP_001167565.1 |
| | | | (isoform d) |
| | | | NM_001174095.1 → NP_001167566.1 |
| | | | (isoform e) |
| | | | NM_001174096.1 → NP_001167567.1 |
| | | | (isoform f) |
| | | | NM_030751.5 → NP_110378.3 |
| | | | (isoform b) |
| Zeb2 | ZEB2 | 9839 | NM_001171653.1 → NP_001165124.1 |
| | | | (isoform 2) |
| | | | NM_014795.3 → NP_055610.1 |
| | | | (isoform 1) |
| Goosecoid | GSC | 145258 | NM_173849.2 → NP_776248.1 |
| FoxC2 | FOXC2 | 2303 | NM_005251.2 → NP_005242.1 |
| Tcf3 | TCF3 | 6929 | NM_001136139.2 → NP_001129611.1 |
| | | | (isoform E47) |
| | | | NM_003200.3 → NP_003191.1 |
| | | | (isoform E12) |
| Klf8 | KLF8 | 11279 | NM_001159296.1 → NP_0052768.1 |
| | | | (isoform 2) |
| | | | NM_007250.4 → NP_009181.2 |
| | | | (isoform 1) |
| FoxC1 | FOXC1 | 2296 | NM_001453.2 → NP_001444.2 |
| FoxQ1 | FOXQ1 | 94234 | NM_033260.3 → NP_150285.3 |
| Six1 | SIX1 | 6495 | NM_005982.3 → NP_005973.1 |
| Lbx1 | LBX1 | 10660 | NM_006562.4 → NP_006553.2 |
| Taz | TAZ | 6901 | NM_000116.3 → NP_000107.1 |
| | | | (isoform 1) |
| | | | NM_181311.2 → |
| | | | NP_851828.1(isoform 2) |
| | | | NM_181312.2 → NP_851829.1 |
| | | | (isoform 3) |
| | | | NM_181313.2 → NP_851830.1 |
| | | | (isoform 4) |
| Yap1 | YAP1 | 10413 | NM_001130145.2 → NP_001123617.1 |
| | | | (isoform 1) |
| | | | NM_001195044.1 → NP_001181973.1 |
| | | | (isoform 3) |
| | | | NM_001195045.1 → NP_001181974.1 |
| | | | (isoform 4) |
| | | | NM_006106.4 → NP_006097.2 |
| | | | (isoform 3) |
| HIF1 | HIF1A | 3091 | NM_001243084.1 → NP_001230013.1 |
| | | | (isoform 3) |
| | | | NM_001530.3 → NP_001521.1 |
| | | | (isoform 1) |
| | | | NM_181054.2 → NP_851397.1 |
| | | | (isoform 2) |
| Sox9 | SOX9 | 6662 | NM_000346.3 → NP_000337.1 |
| Sox10 | SOX10 | 6663 | NM_006941.3 → NP_008872.1 |

In some embodiments, epithelial cells that have been induced to undergo EMT and caused to have increased expression or activity an EMT-cooperating TF exhibit an increase in one or more characteristics associated with stem cells such as self-renewal ability, multi-lineage potential, SC marker expression, sphere-forming ability, organoid formation ability, or organ reconstituting ability, e.g., as compared with control cells. Such characteristics can be assessed using any suitable method known in the art. One of ordinary skill in the art will appreciate that details of appropriate methods may vary depending, e.g., on the particular stem cell or differentiated cell type being assessed. In some embodiments an in vitro method is used. In some embodiments a method may comprise introducing one or more cells into non-human animals and assessing development in vivo of an epithelial outgrowth or organ structure. Control cells can be selected that are sufficiently similar to the cells with which they are compared such that differences in properties assessed would reasonably be attributed to different conditions or agents to which the cells have been exposed rather than differences in intrinsic characteristics of the cells. In some embodiments control cells are from the same species, strain, genetic background, subject, cell line, sample, or preparation as the cells with which they are compared. In some embodiments control cells are epithelial cells that have not been caused to have increased expression or activity of an EMT-cooperating TF and have not been induced to undergo EMT. In some embodiments control cells are epithelial cells that have been caused have increased expression or activity an EMT-cooperating TF but have not been induced to undergo EMT. In some embodiments control cells are epithelial cells that have not been caused to have increased expression or activity an EMT-cooperating TF but have been induced to undergo EMT. In some embodiments, an increase in, e.g., organoid-forming ability or self-renewal ability, is by a factor of at least 2, 5, 10, 20, 50, 100-fold or more.

In some embodiments, cells that have been induced to undergo EMT and caused to have increased expression or activity an EMT-cooperating TF have at least a 5-fold greater ability to migrate or invade, e.g., in vitro, as assessed a migration or invasion assay, than control cells. Assays for migration or invasion are known in the art. See, e.g., Valster A, et al., Methods, 37(2):208-15, 2005. In some embodiments such assays involve a chamber (e.g., a Boyden chamber) consisting of two medium-filled compartments separated by a filter, which may be coated with various components, e.g., ECM components (e.g., Matrigel), in order to assess capacity to invade through such components. A cell suspension is placed in one of the compartments, and incubated. Cells migrate from that compartment through the filter pores to the other side of the filter and are then quantified. If desired, test substances can be included in the medium in either compartment, e.g., to assess the effect of such substances on migration/invasion and/or cells can be exposed to test substances prior to introducing the cells into the chamber.

In some embodiments organ reconstituting ability is assessed by implanting cells into an animal host, e.g., at an orthotopic location, and assessing the ability of the cells to give rise to at least a portion of an epithelial organ or structure, such as a ductal tree. In some embodiments a murine mammary gland reconstitution assay is used, e.g., as described herein.

In some embodiments, cells, e.g., tumor cells, that have been induced to undergo EMT and caused to have increased expression or activity an EMT-cooperating TF show increased resistance to standard chemotherapy drugs (e.g., cytotoxic/cytostatic agents) than control cells. Exemplary methods of assessing resistance or sensitivity to such agents are disclosed, e.g., in WO/2009/126310. In some embodiments such assays comprise contacting cells in culture with a test agent (e.g, a standard chemotherapy agent such as doxorubicin, paclitaxel, etc.), optionally at multiple different concentrations, and assessing viability and/or proliferation of the cells after a time period. Methods for assessing cell viability (survival) and/or proliferation are known to those of ordinary skill in the art. In certain embodiments of any relevant aspect herein, survival and/or proliferation of a cell or cell population, e.g., in cell culture, is determined by: a cell counting assay (e.g., using visual inspection, automated image analysis, flow cytometer, etc.), a replication assay, a cell membrane integrity assay, a cellular ATP-based assay, a mitochondrial reductase activity assay, a BrdU, EdU, or H3-Thymidine incorporation assay, a DNA content assay using a nucleic acid dye, such as Hoechst Dye, DAPI, Actinomycin D, 7-aminoactinomycin D or propidium iodide, a cellular metabolism assay such as resazurin (sometimes known as AlamarBlue or by various other names), MTT, XTT, and CellTitre Glo, etc., a protein content assay such as SRB (sulforhodamine B) assay; nuclear fragmentation assays; cytoplasmic histone associated DNA fragmentation assay; PARP cleavage assay; TUNEL staining; or annexin staining.

In some embodiments, tumor cells that have been induced to undergo EMT and caused to have increased expression or activity an EMT-cooperating TF exhibit increased tumor-initiating ability than control cells. In some embodiments, tumor cells that have been induced to undergo EMT and caused to have increased expression or activity an EMT-cooperating TF exhibit increased metastatic ability than control cells. In some embodiments an increase in tumor-initiating ability and/or in the number of metastases is by a factor of at least 2, 5, 10, 20, 50, 100-fold or more. In some embodiments, non-metastatic tumor cells that have been induced to undergo EMT and caused to have increased expression or activity an EMT-cooperating TF exhibit are rendered capable of forming metastases, e.g., macrometastases.

CSCs and certain SCs often exhibit the ability to form spherical colonies in suspension cultures or soft agar or other semi-solid media. Such colonies are sometimes termed tumorspheres in the case of tumor cells. In the case of mammary SCs, such colonies are sometimes termed mammosphere. Spheres formed by breast tumor cells are sometimes termed tumor mammospheres. In some embodiments epithelial cells that been induced to undergo EMT and caused to have increased expression or activity an EMT-cooperating TF exhibit increased sphere-forming ability as compared with control cells. Exemplary methods of assessing sphere-forming ability are disclosed, e.g., in Dontu, G., et al., (2003) Genes & development 17, 1253-1270; WO/2009/126310 and/or PCT/US2011/049781.

Tumor-initiating ability or metastatic ability can be assessed using methods known in the art, e.g., by introducing cells into a non-human animal host, e.g., an immunocompromised or immunologically compatible non-human host, and observing the number and/or size of resulting tumors. Tumor-initiating ability may be assessed by implanting varying number of tumor cells at an orthotopic location or a non-orthotopic location (e.g., subcutaneously or under the renal capsule would be a non-orthotopic location for tumor types that do not arise naturally in such locations) and determining how many cells are required to give rise to a tumor. Metastatic ability can be assessed by injecting cells into the bloodstream (e.g., into a vein such as a mouse tail vein) and subsequently assessing tumor development (e.g., number, size, average size, total tumor weight or volume, growth rate, etc.) at a distant location such as the lung or implanting tumor cells at an orthotopic or non-orthotopic location in sufficient numbers to give rise to a tumor at said location and subsequently assessing tumor development at a distant location. In some embodiments cells are introduced in or together with a material comprising an extracellular matrix component or hydrogel, which may be isolated from naturally occurring sources or recombinant or chemically synthesized in various embodiments. In some embodiments, the material comprises collagen or Matrigel®. In some embodiments tumor cells are administered as a substantially pure population of tumor cells. In some embodiments tumor cells are mixed with any of various non-cancerous cells. In some embodiments noncancerous cells are fibroblasts or bone marrow derived cells.

In some embodiments an orthotopic or non-orthotopic location of an animal host is “humanized” prior to introduction of human cells (e.g., human normal cells or human tumor cells) into an animal host. For example, in some embodiments a rodent (e.g., mouse, rat) mammary fat pad is a humanized rodent mammary fat pad. Exemplary methods of generating a humanized mammary fat pad are described, e.g., in US Patent Application Publication 20050193432. In some embodiments humanization comprises introducing human stromal elements (e.g., human stromal fibroblasts) into the animal host. For example, in some embodiments mammary stromal fibroblasts (e.g., immortalized human mammary stromal fibroblasts) are introduced into a cleared mammary fat pad.

In some embodiments a non-human animal host is a mammal, e.g., a rodent, e.g., a mouse or rat. In some embodiments an animal host is immunocompromised. Immunocompromised rodent strains are known in the art. For example, in some embodiments a SCID, NOD, NOD-SCID, nude, interleukin-2 receptor-γ (II2rγ)-deficient, Rag1- and/or Rag2 deficient mouse or rat is used. In some embodiments an immunodeficient animal lacks T and/or B cells. In some embodiments, an animal whose thymus gland has been surgically removed or rendered nonfunctional e.g., through a means such as radiation or chemical agents, or whose immune system has been suppressed by drugs or genetic manipulations (e.g., knockdown or knockout of one or more genes that encode molecules important in immune system development and/or function), is used. A subject is “immunologically compatible” with respect to introduced cells (or with respect to a subject from whom such cells originate) if the histocompatibility genes, e.g., major histocompatibility genes of the subject and cells are sufficiently similar such that its immune system does not recognize the cells as foreign and/or does not mount an immune response against the introduced cells. For example, non-human animals of the same inbred strain would generally be immunologically compatible (in the absence of manipulation, e.g., genetic modification, affecting compatibility). In some embodiments, cells introduced into a test animal are of the same species as the test animal into which they are introduced. In some embodiments the cells are isogenic or congenic to the test animal, e.g., the cells originate from an animal of the same inbred strain as the test animal.

In some embodiments of any aspect herein, a difference between two or more values, e.g., a difference relative to a control value, or a result, outcome, or relationship between two or more variables, is statistically significant. In some embodiments “statistically significant” refers to a p-value of less than 0.05 using an appropriate statistical test. One of ordinary skill in the art will be aware of appropriate statistical tests and models for assessing statistical significance, e.g., of differences in measurements, relationships between variables, etc., in a given context. Exemplary tests and models include, e.g., t-test, ANOVA, chi-square test, Wilcoxon rank sum test, log-rank test, Cox proportional hazards model, etc. In some embodiments multiple regression analysis may be used. In some embodiments, a p-value may be less than 0.025. In some embodiments, a p-value may be less than 0.01. In some embodiments, values may be average values obtained from a set of measurements obtained from different individuals, different samples, or different replicates of an experiment. Software packages such as SAS, GraphPad, etc., may be used for performing statistical analysis.

In some embodiments an agent comprises or is modified to comprise or is physically associated with a moiety that enhances cell permeability. In some embodiments a moiety that enhances cell permeability comprises a protein transduction domain (PTD). “Cell permeability” is used interchangeably with “cell uptake” herein and is not intended to imply any particular mechanism. Uptake may comprise traversal of the plasma membrane into the cytoplasm. A PTD is a peptide or peptoid that can enhance uptake by cells, e.g., mammalian cells, of an entity that comprises it or to which it is attached. Many PTDs are known in the art. Exemplary PTDs include various sequences rich in amino acids having positively charged side chains (e.g., guanidino-, amidino- and amino-containing side chains (e.g., U.S. Pat. No. 6,593,292) such as arginine-rich peptides, sequences from HIV Tat protein (e.g., U.S. Pat. No. 6,316,003); penetratin (sequence derived from the homeodomain of Antennapedia); sequences from a phage display library (e.g., U.S. 20030104622); MTS peptide (sequence derived from the Kaposi fibroblast growth factor signal peptide), etc. Organelle-specific PTDs provide a means to target specific subcellular sites, such as the nucleus. See, e.g., Jain M, et al. Cancer Res. 65:7840-7846, 2005; Torchilin V P. Adv Drug Deliv Rev. 58:1532-1555, 2006; Juliano R L, et al. Wiley Interdiscip Rev Nanomed Nanobiotechnol. 1:324-335, 2009; Stewart K M, et al. Org Biomol Chem. 6(13):2242-55, 2008; Fonseca S B, et al., Adv Drug Deliv Rev., 61(11):953-64, 2009; Heitz F, et al., Br J Pharmacol., 157(2):195-206, 2009, and references in any of the foregoing, which are incorporated herein by reference. In some embodiments, a PTD is used to enhance cell uptake of a small molecule, RNAi agent, aptamer, or polypeptide that inhibits an EMT-TF or EMT-cooperating TF. In some embodiments, a PTD is used to enhance cell uptake of an EMT-inducing agent or an EMT-cooperating agent. In some embodiments a polypeptide comprising an EMT-TF or EMT-cooperating TF further comprises a PTD. In some embodiments a PTD is located at the N- or C-terminus. In some embodiments, the polypeptide is a fusion protein.

In some embodiments a stem cell generated from an epithelial cell as described herein gives rise to cells in the normal differentiation pathway of an adult stem cell that initially gave rise to the epithelial cell. For example, in some embodiments a stem cell generated from a mammary luminal epithelial cell gives rise to mammary cell lineages. In some embodiments a stem cell generated herein from an epithelial cell does not (in the absence of further manipulation) give rise to cells outside the normal differentiation pathway of an adult stem cell that initially gave rise to the epithelial cell. For example, in some embodiments a stem cell derived from a mammary luminal epithelial cell gives rise to mammary cell lineages and not to lineages that would normally be found in other organs such as the liver, pancreas, skin, lung, etc. In some embodiments a stem cell generated as described herein from an epithelial cell originating in a particular tissue or organ does not (in the absence of further manipulation such as ectopic expression or other exogenous introduction or activation of one or more additional TFs) transdifferentiate into an adult stem cell characteristically found in a different tissue or organ. In some embodiments a stem cell generated as described herein from an epithelial cell originating in a particular tissue or organ may be further manipulated to cause it transdifferentiate into an adult stem cell characteristically found in a different tissue or organ. In some embodiments a method herein does not reprogram a cell to pluripotency. In some embodiments a method herein may comprise one or more further manipulations of the TF content or expression of a stem cell, wherein the one or more further manipulations reprogram the cell to pluripotency or cause transdifferentiation. In some embodiments a method herein does not comprise one or more further manipulations of the TF content or expression of a stem cell that would reprogram the cell to pluripotency or cause transdifferentiation.

### III. EMT-Cooperating Agents and Methods of Modulation Thereof

In some embodiments an EMT-cooperating TF comprises a Sox protein or a functional variant of any of these. Twenty Sox family proteins have been identified in mice and humans (reviewed in Bernard, P. and Harley, V. R., The International Journal of Biochemistry & Cell Biology 42 (2010) 400-410), The Sox family can be divided into eight groups, A-H (group B is divided into subgroup B1 and B2) based on protein sequence comparisons. Sox proteins belonging to the same group tend to have overlapping tissue expression profiles and are often able to functionally substitute for one another. Within each group, Sox proteins have an overall high degree of amino acid sequence identity (at least 70%), whereas Sox proteins from different groups have a relatively low amino acid sequence identity, particularly outside the HMG box. Sox proteins share more than 50% amino acid identity with the HMG box of the Sox protein SRY. In some embodiments a Sox protein is a SoxE protein. SoxE proteins include Sox8, Sox9, and Sox10.

While cooperation of Sox9 with certain EMT-TFs, e.g., Slug, is demonstrated herein in the Examples in the context of breast epithelial cells, the disclosure encompasses cooperation of Sox9 (and/or other SoxE proteins) with EMT-TFs in other epithelial tissue types. For example, Sox9 is expressed in SCs or progenitor cells in multiple epithelial tissues, including at least the skin, intestine, liver and pancreas (Furuyama et al., 2011; Kopp et al., 2011; Nowak et al., 2008; van der Flier et al., 2009; Vidal et al., 2005). In any aspect herein pertaining at least in part to an EMT-cooperating TF, the disclosure provides embodiments in which the EMT-cooperating TF comprises Sox9, and the epithelial tissue comprises breast, skin, intestine, liver, or pancreas. In some embodiments, one or more EMT-TFs that cooperate with Sox9 (or other SoxE protein) in one or more such tissues is identified. For example, one or more EMT-TFs that is naturally co-expressed with Sox9 (or other SoxE protein) in SCs or progenitor cells can be identified using FISH, immunocytochemistry, or other methods known in the art. In some embodiments the ability of such identified EMT-TF to cooperate with Sox9 is demonstrated by exogenously expressing Sox9 and such EMT-TF in a differentiated epithelial cell of the relevant type and assessing formation of SCs. In some embodiments the ability of such EMT-TFs to cooperate with Sox9 is assessed by inhibiting Sox9 and/or the EMT-TF in a population of SCs of the relevant tissue and assessing one or more SC properties of the resulting cells.

While Sox proteins and the gene expression programs activated by such proteins are exemplified herein, it is envisioned that other TFs and gene expression programs activated thereby may also be capable of cooperating with the EMT to promote formation of stem cells from epithelial cells or to promote maintenance of SCs. By way of example, other TFs that are expressed in various stem cell types and/or during include, e.g., Klf, Myc, Fox, Hes, and catenin proteins. In some embodiments an EMT-cooperating TF is a TF that is endogenously expressed in a stem cell that expresses a particular EMT-TF, i.e., the EMT-TF and EMT-cooperating TF are naturally co-expressed in a stem cell, as exemplified herein with regard to Slug and Sox9.

In some aspects, methods of identifying a TF that cooperates with an EMT-TF of interest are provided. In some embodiments a method of identifying a TF that cooperates with an EMT-TF comprises: (a) assessing expression of one or more candidate TFs in a stem cell that naturally express an endogenous EMT-TF; and (b) identifying a TF that is co-expressed with the EMT-TF in such stem cell. In some embodiments, expression of a set of candidate TFs is assessed, and a TF that is expressed at least 1.5, 2, 5, or 10-fold greater levels than the average expression of the candidate TFs in the set is identified. In some embodiments assessing comprises performing an assay to detect and, optionally, measure, expression of a candidate TF in one or more stem cell types. In some embodiments an assessment of expression is based at least in part on historical data. For example, in some embodiments assessing expression of a TF in stem cells comprises interrogating (e.g., using a computer equipped with or accessing appropriate software) a database comprising gene expression data (e.g., microarray expression data, RNA-Seq expression data) for one or more stem cell types. One of ordinary skill in the art will be aware of numerous TFs whose expression or function as EMT-cooperating TFs can be assessed. As known in the art TFs (sometimes called sequence-specific DNA-binding factors) are protein that bind to specific DNA sequences and (alone or in a complex with other proteins), regulate transcription, e.g., activating or repressing transcription. Exemplary TFs are listed, for example, in the TRANSFAC® database, Gene Ontology (http://www.geneonlology.org/) or DBD (www.transcriptionfactor.org) (Wilson, et al, DBD—taxonomically broad transcription factor predictions: new content and functionality Nucleic Acids Research 2008 doi:10.1093/nar/gkm964). As known in the art, TFs can be classified based on the structure of their DNA binding domains (DBD). For example in certain embodiments a TF is a helix-loop-helix, helix-turn-helix, winged helix, leucine zipper, bZIP, zinc finger, homeodomain, or beta-scaffold factor with minor groove contacts protein. In some embodiments a TF is not a general transcription factor (also termed a basal transcription factor). In some embodiments a TF is one that is normally developmentally expressed in a mammalian organism (e.g., expression begins post-blastocyst stage). In some embodiments a TF is one that is normally first expressed in a mammalian organism during or following gastrulation. In some embodiments a TF is one that is normally first expressed in a mammalian organism during organogenesis. In some embodiments a TF is at least somewhat cell type specific. In some embodiments a TF is, under normal conditions, naturally expressed selectively in one or more tissues or cell types of ectodermal, endodermal, or mesodermal origin. In some embodiments a TF is one that is not naturally expressed in embryonic stem (ES) cells. In some embodiments a TF is one that is naturally embryonic stem (ES) cells. In some embodiments a TF is not one known in the art to be of use to generate induced pluripotent stem (iPS) cells.

As known in the art, iPS cells are pluripotent cells that are derived from non-pluripotent cells (e.g., adult somatic cells such as fibroblasts) by methods such as causing such cells to express certain TFs, sometimes termed “reprogramming factors” selected from, e.g., Oct4, Sox2, Nanog, Lin28, Klf4, and Myc (see, e.g., WO/2008/124133 and references therein). In some embodiments a TF, e.g., an EMT-cooperating TF or EMT-TF, is not Oct4, Sox2, Nanog, Lin28, Klf4, or Myc or a TF that can substitute for one or more of the foregoing in reprogramming a somatic cell to pluripotency.

In some embodiments a method of identifying a TF that cooperates with an EMT-TF comprises: (a) ectopically co-expressing a first protein comprising a candidate TF and a second protein that comprises an EMT-TF in a differentiated epithelial cell; and (b) comparing the cell of step (a) with a control cell with respect to one or more stem cell properties, wherein if the cell of step (a) exhibits increased stem cell properties as compared with the control cell, the candidate TF is identified as an EMT-cooperating TF. In some embodiments the control cell is an epithelial cell of the same type as the cell of step (a), wherein the control cell ectopically expresses the EMT-TF but does not ectopically express the candidate TF. In some embodiments a method of identifying a TF that cooperates with an EMT-TF comprises: (a) ectopically expressing a protein comprising a candidate TF in a cell that expresses an EMT-TF; and (b) determining whether the cell acquires one or more stem cell properties, wherein if the cell acquires one or more stem cell properties, the candidate TF is identified as an EMT-cooperating TF. In general, cell can be assessed with regard to any one or more stem cell properties some embodiments determining whether the cell acquires one or more stem cell properties. Exemplary stem cell properties and methods of assessment thereof are described elsewhere herein. In some embodiments an EMT-cooperating TF or candidate TF comprises a Klf, Myc, Fox, Hes, or catenin protein (e.g., β-catenin) or a functional variant of any of these.

It is also envisioned that various proteins whose expression is activated by an EMT-cooperating TF and/or that are expressed in stem cells or during development may be capable of cooperating with EMT-TFs to promote formation or maintenance of SCs. Thus in some aspects, a method of generating stem cells from epithelial cells comprises steps of: (a) providing a population of epithelial cells; and (b) inducing EMT and increasing the amount or activity of at least one EMT-cooperating protein in the population of epithelial cells, thereby generating stem cells in the population. In some embodiments the EMT-cooperating protein is a protein whose expression is activated by an EMT-cooperating TF. In some embodiments an EMT-cooperating protein is a transcriptional co-activator, co-repressor, chromatin remodeler, acetylase, deacetylase, kinases, and methylase. In some embodiments an acetylase or methylase acts on DNA. In some embodiments an acetylase or methylase acts on histone(s).

In some embodiments expression of an endogenous EMT-cooperating protein, e.g., an endogenous EMT-TF, is increased by inhibiting one or more microRNAs (miRNAs) that would otherwise inhibit expression of the EMT-cooperating TF. Exemplary Methods for modulating miRNA expression or activity are described above.

IV. EMT Induction and EMT-Inducing Agents

In general, EMT can be induced by any of a variety of different methods. Such methods may make use of any of a variety of different EMT-inducing agents. Exemplary methods of inducing EMT and exemplary EMT-inducing agents are disclosed, e.g., in PCT/US2006/025589 (published as WO2007/005611); PCT/US2009/002254 (published as WO/2009/126310); PCT/US2011/049781; Zavadil J et al., Oncogene 24: 5764-5774, 2005; Sato M, J Clin Invest 112: 1486-1494, 2003; Gregory P A, et al., Nat Cell Biol. Mar. 30, 2008; Zeng R, et. al., J Am Soc Nephrol. 2008 February; 19(2):380-7. Epub 2008 Jan. 9; Krawetz R, et al., Cell Signal. 2008 March; 20(3):506-17; Jiang Y G, et al., Int J Urol. 2007 Nov. 14(11):1034-9; Lo H W, et al., Cancer Res. 2007 Oct. 1, 67(19):9066-76; Lester R D, et al., J Cell Biol. 2007 Jul. 30 178(3):425-36; Moustakas A, et al., Cancer Sci. 2007 October 98(10):1512-20; Wahab N A, et al., Nephron Exp Nephrol. 2006, 104(4):e129-34. It will be understood that these methods are not meant to be limiting and other appropriate methods will be apparent to one of ordinary skill in the art. It will also be understood that in some embodiments two or more methods or agents may be used in combination. In some embodiments a composition for inducing EMT comprises two or more EMT-inducing agents.

In some embodiments EMT is brought about by causing epithelial cells to express one or more EMT-TFs. EMT-TFs include, e.g., Slug, Snail, Twist1, Twist2, Zeb1, Zeb2, Goosecoid, FoxC2, Tcf3, Klf8, FoxC1, FoxQ1, Six1, Lbx1, Yap1, and HIF-1. In some embodiments EMT is brought about by causing epithelial cells to express a polypeptide comprising Slug, Snail, Twist1, Twist2, Zeb1, Zeb2, Goosecoid, FoxC2, Tcf3, Klf8, FoxC1, FoxQ1, Six1, Lbx1, Yap1, and HIF-1 or a functional variant of any of these. In certain embodiments a functional variant of a protein comprises a polypeptide at least 90%, 91%, 92%, 93%, 94%, 95%, 96%, 97%, 98%, 99%, or more identical to the protein. In some embodiments EMT is brought about by activating a signaling pathway that leads to expression of a gene encoding an EMT-TF or activates an EMT-TF. In some embodiments EMT is brought about by inhibiting a signaling pathway or molecule that inhibits expression of a gene encoding an EMT-TF or inhibits activity of an EMT-TF. In some embodiments EMT is brought about by causing epithelial cells to express a polypeptide comprising the transcriptional co-activator Taz or a functional variant thereof.

In some embodiments EMT is brought about by manipulating miRNA expression or activity. For example, in some embodiments EMT is brought about by inhibiting expression or activity of a miRNA that would otherwise inhibit expression of an EMT-TF. For example, the miR-200 family and miR-205 regulate epithelial-to-mesenchymal transition by targeting ZEB1 and SIP1 (Gregory P A, et al., Nat Cell Biol. 2008 May; 10(5):593-601). In some embodiments inhibiting one or more of such miRNAs is used to induce EMT. Activity of a miRNA can be inhibited by various approaches such as introducing into a cell an oligonucleotide complementary to the miRNA (such an oligonucleotide is sometimes referred to as an “antagomir”) or an oligonucleotide complementary to a target region of an mRNA (such an oligonucleotide is sometimes termed a “target protector”). “Target region” refers to that portion of a target mRNA to which a miRNA would otherwise bind. In some embodiments expression of a miRNA is inhibited by causing a cell to contain or express a miRNA sponge. MicroRNA sponges are transcripts with multiple sequences antisense to at least a portion of an miRNA (e.g., antisense to at least a seed region of a miRNA) that can bind to miRNAs and thereby sequester them from endogenous or ectopic targets. (See Ebert M S, Sharp PA.RNA. 2010 November; 16(11):2043-50 for review.) In some embodiments a miRNA sponge comprises 5, 10, or more miRNA binding sites, which may be identical or different in various embodiments. In some embodiments a miRNA sponge inhibits activity of multiple miRNAs of an miRNA family. In some embodiments a miRNA sponge comprises binding sites for multiple different miRNAs, e.g., miRNAs that would bind to different target regions of an endogenous transcript, or that would bind to different endogenous transcripts. In various embodiments an miRNA sponges is expressed intracellularly using transient or stable expression methods.

In some embodiments, EMT is brought about by modulating the activity of a signaling pathway in a cell, wherein the signaling pathway is selected from TGF-β, Wnt, BMP, Notch, HGF-Met, EGF, IGF, PDGF, FGF, P38-mapk, Ras, PB Kinase-Akt, Src, and NF-kB. In some embodiments, the signaling pathway that induces EMT is modulated by contacting a cell with a growth factor selected from: a TGF-β superfamily member, a Wnt-family member, an FGF family member, a Notch ligand, a Hedgehog ligand, an EGF family member, an IGF family member, PDGF, and HGF. In some embodiments, the signaling pathway that induces EMT is modulated by contacting a cell with TGF-β1. Exemplary TGF-β superfamily members include TGF-βl, TGF-β2, TGF-β3, BMP2, BMP3, BMP4, BMP5, BMP6, BMP7, BMP8a, BMP8b, BMPoO, BMP15, GDFl, GDF2, GDF3, GDF5, GDF6, GDF7, Myostatin/GDF8, GDF9, GDFlO, GDFl 1, GDFl 5, Activin A and B/Inhibin A and B, Anti-müllerian hormone, and Nodal. Exemplary FGF family members include FGFl, FGF2, FGF4, FGF8, FGF10. Exemplary Wnt-family members include WNT1, WNT2, WNT2B/13, WNT3, WNT3A, WNT4, WNT5A, WNT5B, WNT6, WNT7A, WNT7B, WNT8A, WNT8B, WNT9A, WNT9B, WNT10A, WNT10B, WNT11, and WNT16. Exemplary EGF family members include epidermal growth factor (EGF), heparin-binding EGF-like growth factor (HB-EGF), transforming growth factor-α (TGF-α), Amphiregulin (AR), Epiregulin (EPR), Epigen, betacellulin (BTC), neuregulin-1 (NRGl), neuregulin-2 (NRG2), neuregulin-3 (NRG3), and neuregulin-4 (NRG4). Exemplary IGF family members include IGF1 and IGF2. In some embodiments a small molecule or peptide is used to stimulate TGF signaling. For example, PCT/US2008/011648 (WO/2009/051660) discloses small molecules reported to activate TGF beta signaling. In some embodiments, one or more small molecules that act on proteins involved in one or more steps of the Wnt signaling pathway are used. For example, GSK3 inhibitors may be used to activate canonical Wnt signaling. Many potent and selective small molecule inhibitors of GSK3 have been identified. See, e.g., Wagman A S, Johnson K W, Bussiere D E, Curr Pharm Des., 10(10): 1 105-37, 2004, for some examples. One of ordinary skill in the art will be aware of others.

In certain embodiments EMT is brought about by inhibiting the expression or activity of E-Cadherin in the cell. The expression or activity of E-Cadherin can be inhibited by using methods known to one of ordinary skill in the art. Exemplary methods for inhibiting E-cadherin expression or activity include contacting a cell with a small interfering nucleic acid complementary to E-Cadherin mRNA, contacting a cell with a blocking antibody to E-cadherin; inducing the expression of dysadherin, for example by cDNA-based overexpression of dysadherin, in a cell; and interfering with cell-polarity genes in the cell. For example, depletion of Scribble disrupts E-cadherin-mediated cell-cell adhesion and induces EMT (Qin Y, et al., J Cell Biol 2005; 171:1061-71). Thus, in some embodiments, inhibition of Scribble, PAR, or crumbs (CRB) such as by RNA interference (RNAi), can induce an EMT. In some embodiments a gene affecting cell polarity whose inactivation results in loss of E-cadherin expression is inhibited (e.g., a mammalian counterpart of a gene identified in Pagliarini R A, et al., Science 2003; 302:1227-31). Other exemplary methods for interfering with cell polarity genes to induce EMT are known in the art. In some embodiments EMT is brought about by inhibiting expression or activity of one or more other adhesion complex proteins. For example, in some embodiments expression or activity of Occludin, Claudin 1, or Claudin 2 (tight junction proteins with extracellular domains) is inhibited. In some embodiments, a protease that cleaves an adhesion complex protein is used. For example, a matrix metalloprotease (MMP) or calpain is used in some embodiments.

Various strategies for gene knockdown known in the art can be used to inhibit the expression of a gene, for example E-cadherin and/or other genes disclosed herein, inhibition of which is useful for inducing EMT. In certain embodiments expression of a gene, e.g., E-cadherin, is inhibited by RNAi. Methods for inhibiting gene expression, such as E-cadherin expression, by RNAi are known in the art. In some embodiments, a cell is transfected with a small interfering nucleic acid complementary to E-Cadherin mRNA in the cell to inhibit E-cadherin activity in the cell. Exemplary small interfering nucleic acids are known to those of ordinary skill in the art. Methods for transfection of small interfering nucleic acids (e.g., siRNA) are well known in the art. In some embodiments, the cell has a stably integrated transgene that expresses a small interfering nucleic acid (e.g., shRNA, miRNA) that is complementary to E-cadherin mRNA and that causes the downregulation of E-cadherin mRNA through the RNA interference pathway. For example, gene knockdown strategies may be used that make use of RNAi pathways including, e.g., use of small interfering RNA (siRNA), short hairpin RNA (shRNA), double-stranded RNA (dsRNA), miRNAs, and other nucleotide-based molecules known in the art. In some embodiment, vector-based RNAi modalities (e.g., shRNA or miRNA precursor-based expression constructs) are used to reduce expression of a gene in a cell.

In some embodiments EMT is brought about using an antibody, aptamer, or other binding agent to inhibit one or more proteins, such as E-cadherin or another adhesion junction protein. In some embodiments cells are cultured in medium comprising the agent for a sufficient time and in a sufficient amount to induce EMT.

In some embodiments inducing EMT comprises inhibiting one or more molecules that may otherwise inhibit or oppose EMT. For example, certain cells produce endogenous inhibitors of the Wnt pathway, which inhibitors may inhibit such cells or other cells in the same culture vessel or environment from undergoing EMT. In some embodiments an agent such as an antibody, aptamer, or RNAi agent is used to inhibit expression or activity of one or more such inhibitors. See, e.g., PCT/US2011/049781. In some embodiments stimulation of the TGFβ pathway and of canonical and non-canonical Wnt pathways and restriction of BMP pathway signaling can collaborate in inducing EMT. In some embodiments inducing EMT comprises reducing the levels of secreted endogenous inhibitors of the TGFβ and/or Wnt pathway(s), e.g., SFRP1, DKK1, and BMPs. In some embodiments, induction or maintenance of EMT is facilitated by perturbing cell adhesion, e.g., by perturbing adherens junction formation or maintenance (e.g., inhibiting E-cadherin expression or activity), in combination with stimulating TGFβ and/or Wnt pathway(s).

In some embodiments inducing an EMT comprises (a) stimulating TGF-β pathway signaling; (b) stimulating canonical Wnt pathway signaling; (c) stimulating non-canonical Wnt pathway signaling; and/or (d) perturbing cell adhesion. In some embodiments stimulating TGF-β pathway signaling comprises providing an extracellular environment that is permissive for TGF-β signaling. In some embodiments, an environment that is permissive for TGF-β signaling is one in which BMP pathway signaling is inhibited. In some embodiments, inhibiting BMP pathway signaling comprises downregulating synthesis of one or more endogenous BMP ligands that would otherwise stimulate BMP signaling or providing a BMP antagonist.

In some embodiments, EMT is brought about by subjecting a cell to a stress selected from: hypoxia, irradiation, and chronic chemotherapy treatment. Methods for inducing cell stress are known in the art. Exemplary methods are disclosed in Docherty, N G, et al., Am J Physiol Renal Physiol 290: F1202-F1212, 2006 and Manotham K, et al., Kidney Int 65: 871-880, 2004.

If desired, cells can be assessed the cells for evidence of EMT using any of a variety of methods. One could examine, e.g., induction of EMT-TFs such as Zeb1, Zeb2, Twist, etc., and/or mesenchymal markers such as N-Cadherin, vimentin, etc. For example, in some embodiments, upregulation of at least one EMT-associated TF, e.g., by at least 5-fold, 10-fold, 20-fold, 50-fold, or 100-fold in a population of cells indicates EMT. In some embodiments, downregulation of at least one EMT-associated TF, e.g., by at least 5-fold, 10-fold, 20-fold, 50-fold, or 100-fold in a population of cells indicates a reversal of EMT. One could alternately or additionally examine properties such as motility or capacity for self-renewal that are increased in cells that have undergone an EMT. One could alternately or additionally determine the extent to which cells exhibit alteration (reduction or increase) in epithelial characteristics. For example, cells that have undergone EMT exhibit reduced expression of markers such as E-cadherin, epithelial cytokeratins, etc. In some embodiments, expression of a marker is reduced by at least 5-fold, 10-fold, 20-fold, or more in cells that have undergone EMT. In some embodiments epithelial cells are CD44^{low} and CD24^{high} prior to undergoing EMT while cells that have undergone an EMT are CD44^{high} and CD24^{low}.

### V. Cells and Markers

Epithelial cells (or other cells) of use in compositions and methods described herein and/or to which methods described herein are applied, can be obtained from any of a wide variety of sources or, in the case of certain in vivo applications, may be present in a variety of tissues or organs. In various embodiments epithelial cells may originate from any epithelial tissue. One of skill in the art will appreciate that “epithelium” refers to layers of cells that line the cavities and surfaces of structures throughout the body and is also the type of tissue of which many glands are at least in part formed. Such tissues include, for example, tissues found in the breast, gastrointestinal tract (stomach, small intestine, colon), liver, biliary tract, bronchi, lungs, esophaghus, pancreas, kidneys, ovaries, prostate, skin, cervix, uterus, vagina, bladder, ureter, testes, exocrine glands, endocrine glands, eye, nose, mouth, etc. In some embodiments the epithelium is endothelium or mesothelium. In certain embodiments cells are human breast epithelial cells. In some embodiments cells are noncancerous human cells. In some embodiments cells are noncancerous human breast cells obtained, e.g., from a reduction mammoplasty. In certain embodiments, epithelial cells are of a cell type that normally expresses E-cadherin. In certain embodiments, epithelial cells are of a cell type that does not normally express N-cadherin. In certain embodiments, epithelial cells are of a cell type that normally expresses E-cadherin at levels at least 5, 10, 20, 50, or 100-fold higher levels, on average, than those at which it expresses N-cadherin. Cells derived from mammary tissue are exemplified herein, but it will be understood that embodiments pertaining to cells derived from other tissues are encompassed. In some embodiments, cells are isolated cells.

In some embodiments cells are of a cell type found in a gland. In some embodiments a gland comprises a glandular portion, which produces secretions, and a duct portion that channels secretions towards the external environment. In some embodiments a gland comprises luminal cells and basal cells. Luminal cells comprise a layer of cells located adjacent to a lumen of the gland. In some embodiments a progenitor cell is a luminal progenitor cell. Basal cells are located external to the luminal cells (i.e., further away from the lumen). In at least some portions (e.g., ducts) of certain glands, basal cells form a layer at the basal surface of the epithelium adjacent to the basement membrane. In some embodiments basal cells comprise myoepithelial cells. As will be appreciated by one of ordinary skill in the art, differentiated myoepithelial cells resemble smooth muscle cells in certain respects. For example, they have contractile properties and express various smooth muscle-specific contractile and cytoskeletal proteins (e.g., smooth muscle actin). Myoepithelial cells are considered epithelial cells because, e.g., the major components of their intermediate filament system are various cytokeratins, they form desmosomes, hemidesmosomes and cadherin-mediated cell-cell junctions, and in vivo they are separated from underlying connective tissue by a basement membrane. As described herein, myoepithelial cells typically exhibit certain mesenchymal characteristics such as endogenously expressing one or more mesenchymal markers. In some embodiments a progenitor cell is a myoepithelial progenitor cell.

In various embodiments cells can be primary cells, untransformed cells, transformed cells, non-immortalized cell lines, immortalized cell lines, transformed immortalized cell lines, benign tumor derived cells or cell lines, malignant tumor derived cells or cell lines, transgenic cell lines. In some embodiments cells are non-genetically modified cells. In some embodiments cells are genetically modified. In some embodiments, cells are maintained in culture and passaged or allowed to double once or more following their isolation from an individual (e.g., between 2-5, 5-10, 10-20, 20-50, 50-100 times, or more) prior to their use in a method disclosed herein. In some embodiments, cells have been passaged or permitted to double no more than 1, 2, 5, 10, 20, or 50 times following their isolation from an individual prior to their use in a method disclosed herein.

In various embodiments cells, e.g., epithelial cells, can originate from any mammalian organism, e.g., a human, non-human primate, rodent (e.g., mouse, rat, guinea pig, hamster, rabbit), cow, sheep, goat, pig, etc. Methods useful for obtaining cells, and suitable sources, are known to those of ordinary skill in the art. In some embodiments cells are obtained from a biopsy (e.g., tissue biopsy, fine needle biopsy, etc.) or at surgery for a noncancerous condition or for cancer. In some embodiments cells are obtained from a subject, e.g., a subject who is expected to be a future recipient of cells derived from the obtained cells or a relative or immunologically compatible donor. A situation in which cells removed from a subject, or descendants thereof, are subsequently introduced into the subject may be referred to as an “autologous”. Introduced cells may be referred to as a “graft” or “transplant”. In some embodiments of any aspect herein pertaining at least in part to cell transplantation, human cells are transplanted into a human subject. In some embodiments of any aspect herein pertaining at least in part to cell transplantation human cells are transplanted into a non-human subject. In some embodiments of any aspect herein pertaining at least in part to cell transplantation non-human cells are transplanted into a human subject. In some embodiments cells may be obtained from discarded surgical or cellular samples from a subject. Mammary tissue is a useful source of cells in certain embodiments. For example, primary human mammary epithelial cells (MECS) can be derived from fresh breast reduction tissue (reduction mammoplasty) by mechanical dissociation and, if desired, can be further purified by methods such as fluorescence activated cell sorting (FACS). In some embodiments similar approaches are used to isolate epithelial cells from other tissues. Primary MECS (or other epithelial cell types) can be genetically modified through introduction of various genetic elements, such as vectors (e.g., retroviral vectors) encoding the catalytic subunit of the human telomerase holoenzyme (hTERT) to generate immortalized cell lines. In some embodiments such a cell line is further genetically modified and transformed to convert it into a tumor cell.

In some embodiments cells are identified, isolated, or classified based at least in part on expression of one or more markers. In some embodiments a marker is a cell surface marker. In some embodiments a measurement of the expression of one or more markers is used to assess whether a stem or progenitor cell has been generated. In some embodiments measurement of the expression of one or more markers is used to assess whether a cell of a particular differentiated cell type has been generated. It will be appreciated that marker patterns of cells can be readily determined by techniques, such as flow cytometry, e.g., cell fluorescence-activated cell sorting and immunohistochemistry, etc. As will be understood, with respect to cell markers and their expression levels, “neg” (−) or “low” refers to the absence or negligible or low level of expression of the marker, and “pos” (+) or “high” refers to robust expression. A transition of expression of a cellular marker from “neg” to “pos” represents a change from the lack of expression or low levels of expression to a high level or much higher level of expression. Thus “low” refers to a low level, “high” refers to an easily detectable and high level of expression, and the distinction between low and high expression and/or the transition from low to high expression levels, or from high to low expression levels, would be readily apparent to the practitioner. It will also be understood that in the case of certain markers activity assays, if available, can be used instead of or in addition to measurement of measuring the level of mRNA or protein.

In some embodiments a marker for normal epithelial cells is a claudin. Claudins are members of a large family of 27 closely related transmembrane proteins that play a crucial role in formation, integrity and function of tight junctions. In some embodiments a marker is a cell adhesion molecule. In some embodiments a marker is an integrin. In some embodiments a marker is a cytokeratin.

In some embodiments a stem cell, e.g., a human mammary stem cell, is CD44+/CD24−. In some embodiments a differentiated epithelial cell, e.g., a differentiated human mammary epithelial cell, is CD44−/CD24+. In certain embodiments, one or more cellular markers are selected from: CD10, CD15, CD20, CD24, CD34, CD38, CD44, CD45, CD105, CD133, CD166, CD171 (L1CAM), EpCAM, ESA, SCA1, Pecam, Stro1, alpha 6 integrin, and ALDH. These markers are non-limiting examples of markers that may be used in various embodiments. Markers appropriate for stem, progenitor, or differentiated epithelial (or non-epithelial) cells of different organs or tissues can be selected by one of ordinary skill in the art.

In some embodiments a basal cell, e.g., a mammary basal cell, expresses P63deltaN, ID4, Egr2, Mef2C, Tbx2, and/or an EMT-TF. In some embodiments one or more such genes are signature genes for a basal cell lineage. In some embodiments a luminal progenitor cell, e.g., a mammary luminal progenitor cell, expresses c-Kit, Elf5, CXCR4, LBP, or Sox10. In some embodiments one or more such genes are signature genes for a luminal cell lineage.

In some embodiments human mammary cells are identified, isolated, or classified based at least in part on expression of CD49f and/or EpCam. For example, human mammary stem cells are CD49f-high/EpCAM-low; human mammary luminal progenitor cells are CD49f-high/EpCAM-high, and differentiated human mammary luminal cells are CD49f-low/EpCAM-high.

One of ordinary skill in the art will understand that the particular gene expression patterns of luminal and/or basal progenitor cells may differ between different species and/or organs. In some embodiments a stem cell property comprises expression of one or more markers characteristic of stem cells. In some embodiments a stem cell property comprises low expression or absence of expression of one or more markers characteristic of differentiated cells. Thus in some embodiments determining whether a cell exhibits one or more stem cell properties comprises assessing expression of one or more stem cell markers by the cell.

It will be understood that if an exogenously introduced nucleic acid is used to induce EMT, to activate a gene expression program, or to generate a stem cell, then expression of one or more endogenous genes (e.g., one or more genes encoding expression products distinct from those encoded by the exogenously introduced nucleic acid), or other means of assessment, may be used to determine whether EMT has been induced, whether a gene expression program of interest has been activated, or whether a stem cell has been generated. For example, if an exogenously introduced nucleic acid encoding an EMT-TF such as Slug is introduced, expression of E-cadherin may be assessed to determine whether EMT has been induced. In some embodiments determining whether a cell exhibits one or more stem cell properties comprises assessing the ability of the cell to give rise to cells of at least two distinct cell types. In some embodiments determining whether a cell exhibits one or more stem cell properties comprises assessing the ability of the cell to give rise to an organoid.

In some embodiments cells are tumor cells. In some embodiments a tumor cell has been obtained or derived from a tumor arising in a subject. In some embodiments a tumor cell is generated by genetic modification of a non-tumor cell. In some embodiments a tumor cell, tumor cell line, or tumor comprises one or more oncogenes or has reduced or absent expression of one or more tumor suppressor genes (TSGs) or reduced or absent activity of one or more TSG gene products, e.g., as a result of a mutation in the TSG. The term “oncogene” encompasses nucleic acids that, when expressed, can increase the likelihood of or contribute to cancer initiation or progression. Normal cellular sequences (“proto-oncogenes”) can be activated to become oncogenes (sometimes termed “activated oncogenes”) by mutation and/or aberrant expression. In various embodiments an oncogene can comprise a complete coding sequence for a gene product or a portion that maintains at least in part the oncogenic potential of the complete sequence or a sequence that encodes a fusion protein. Oncogenic mutations can result, e.g., in altered (e.g., increased) protein activity, loss of proper regulation, or an alteration (e.g., an increase) in RNA or protein level. Aberrant expression may occur, e.g., due to chromosomal rearrangement resulting in juxtaposition to regulatory elements such as enhancers, epigenetic mechanisms, or due to amplification, and may result in an increased amount of proto-oncogene product or production in an inappropriate cell type. As known in the art, proto-oncogenes often encode proteins that control or participate in cell proliferation, differentiation, and/or apoptosis. These proteins include, e.g., various transcription factors, chromatin remodelers, growth factors, growth factor receptors, signal transducers, and apoptosis regulators. Oncogenes also include a variety of viral proteins, e.g., from viruses such as polyomaviruses (e.g., SV40 large T antigen) and papillomaviruses (e.g., human papilloma virus E6 and E7). A TSG may be any gene wherein a loss or reduction in function of an expression product of the gene can increase the likelihood of or contribute to cancer initiation or progression. Loss or reduction in function can occur, e.g., due to mutation or epigenetic mechanisms. Many TSGs encode proteins that normally function to restrain or negatively regulate cell proliferation and/or to promote apoptosis when appropriate. In some embodiments an oncogene or TSG encodes a miRNA. Exemplary oncogenes include, e.g., MYC, SRC, FOS, JUN, MYB, RAS, RAF, ABL, ALK, AKT, TRK, BCL2, WNT, HER2/NEU, EGFR, MAPK, ERK, MDM2, CDK4, GLI1, GLI2, IGF2, TP53, etc. Exemplary TSGs include, e.g., RB, TP53, APC, NF1, BRCA1, BRCA2, PTEN, CDK inhibitory proteins (e.g., p16, p21), PTCH, WT1, etc. It will be understood that a number of these oncogene and TSG names encompass multiple family members and that many other TSGs are known. In various embodiments non-tumor cells can be converted to tumor cells by genetically modifying the cells to express an oncogene and/or to lack expression of a TSG, or a cancer-prone non-human animal (which animal may be of use as a source of tumor cells or for testing a candidate anti-tumor agent) can be generated through genetic modification causing at least some of the cells of the animal to express one or more oncogenes and/or to have reduced expression of one or more TSGs. Methods and suitable combinations of oncogenes and/or TSG knockouts for such purposes are known in the art.

### VI. Compositions, Culture Media, Culture Systems, and Kits

In some aspects, the disclosure provides a composition comprising: (a) an EMT-inducing agent; and (b) an EMT-cooperating agent. In some embodiments a composition further comprises: (c) one or more cell culture medium components. In some embodiments the EMT-cooperating agent comprises, encodes, or induces expression of a polypeptide comprising at least one Sox protein.

Any of a variety of cell culture media components or cell culture media could be used. One of skill in the art will be aware of the components of numerous culture media and methods for preparation thereof, such as nutrients (e.g., sugars and amino acids), vitamins, trace elements, ions, lipids, hormones, growth factors, etc. See, e.g., Freshney, supra. Exemplary cell culture media include, e.g., MEGM, DMEM, Ham's F-12, Epicult-B, and mixtures thereof. One of ordinary skill in the art would appreciate that the precise amounts of many of the various components of a cell culture medium could be varied without adversely affecting the ability of the medium to support cell growth. For example, a medium may contain between 0.1 and 100-fold the concentration of any one or more components, in various embodiments. In some embodiments, the culture medium is suitable for culturing an epithelial cell type of interest. In some embodiments the composition comprises (a) an EMT-inducing agent; and (b) an agent that comprises, encodes, or induces expression of a polypeptide comprising at least one Sox protein or enhances activity of a polypeptide comprising at least one Sox protein; and (c) cell culture medium components sufficient to form a cell culture medium capable of supporting growth of at least one differentiated epithelial cell type or epithelial progenitor cell type for at least 5, e.g., at least 10 population doublings.

In some aspects, the disclosure provides methods of obtaining or culturing organoids. In some embodiments a method of obtaining an organoid comprises culturing an epithelial stem cell in a composition comprising about 5% Matrigel. In some embodiments a method of maintaining an organoid comprises culturing an organoid in a composition comprising about 5% Matrigel. In some embodiments a composition comprising about 5% Matrigel comprises no more than about 0.1%, 0.2%, 0.5%, or 1% by volume of other components that would increase the viscosity of the composition, such as cellulose, methylcellulose or other cellulose derivatives, agar, agarose, or gel-forming synthetic organic polymers. In some embodiments a composition for obtaining, maintaining, or analyzing organoids comprises a Rho-associated kinase (ROCK) inhibitor. In some embodiments a ROCK inhibitor is Y-27632, Y-39983, thiazovivin, fasudil, GSK429286A, HA-1077 (1-(5-Isoquinolinylsulfonyl)homopiperazine dihydrochloride), H-1152P ((S)-(+)-2-methyl-1-[(4-methyl-5-isoquinolinyl) sulfonyl]homopiperazine) or an analog of any of these compounds. In some embodiments a composition useful for generating or maintaining stem cells or converting an epithelial cell into a less differentiated cell disclosed herein comprises a ROCK inhibitor. Numerous ROCK inhibitors are known. In various embodiments, any ROCK inhibitor can be used. Exemplary ROCK inhibitors are described in, e.g., WO/2011/107608; WO/2007/026920; WO/2007/133622; WO/2004/041813; WO/2011/130740; WO/2007/060028; WO/2007/060028; WO/2009/126635; WO/2005/105780; WO/2005/103050; WO/2005/019190, among others. In some embodiments a ROCK inhibitor inhibits both ROCK1 and ROCK2. In some embodiments a ROCK inhibitor is relatively selective for ROCK1 and/or ROCK2. For example, the IC50 for the compound may be at least 5-, 10-, or 20-fold lower for inhibiting ROCK1 and/or ROCK2 than for inhibiting at least 90%, 95%, 99%, or more other kinases (e.g., protein kinases) in the kinome. In some embodiments a ROCK inhibitor inhibits at least one non-ROCK kinase. In some embodiments the additional kinase is GSK3. In some embodiments the composition comprises about 5 μM Y-27632 or an appropriate amount of a different ROCK inhibitor to achieve at least substantially the same effect. In some embodiments a ROCK inhibitor is used at a concentration sufficient to reduce activity of one or more ROCK proteins by at least about 10%, 25%, 50%, or more. In some embodiments a ROCK inhibitor is not used or, if used, is used at a concentration that reduces activity of one or more ROCK proteins by no more than about 1%, 5%, or 10%.

In some embodiments a composition comprises about 5% Matrigel, about 5% serum, about 5-25 ng/ml EGF (e.g., about 10 ng/ml EGF), and about 10-50 ng/ml bFGF (e.g., about 20 ng/ml bFGF). In some embodiments the composition further comprises about 2-10 μg/ml heparin, e.g., about 4-5 μg/ml heparin. In some embodiments serum comprises fetal bovine serum.

In some embodiments a method of assessing the differentiation potential of a cell comprises: (a) culturing the cell in a composition comprising about 5% Matrigel; and (b) assessing the ability of the cell to give rise to an organoid. In some embodiments the method comprises assessing at least one property of the organoid. For example, in some embodiments the method comprises determining whether the organoid is hollow or solid. In some embodiments the method comprises seeding single cells in each of one or more vessels and counting the number of organoids formed after a selected time period in at least one vessel. In some embodiments the method comprises seeding multiple cells in each of one or more vessels and counting the number of organoids formed after a selected time period in at least one vessel. In some embodiments a time period is between 3 and 21 days, e.g., between 5 and 18 days, e.g., between 7 and 14 days. In some embodiments cells are seeded at between 1 and 10,000 cells, e.g., between 50 and 5,000, e.g., between 100 and 2,500, e.g., between 1000-2000 cells in a volume of between about 100 μl-250 μl medium, or at approximately equivalent density in another suitable culture vessel. One of ordinary skill in the art could perform experiments at a range of cell densities to determine the optimal cell seeding number. In some embodiments cells are seeded in wells of a 96-well plate. In some embodiments cells are seeded in wells of a 6, 12, 24, 96, 384, or 1536-well plate. In some embodiments cells are seeded in a vessel that has a surface designed for low cell attachment. For example, ultra-low attachment plates may be used (e.g., available from Corning). In some embodiments organoids at least 50, at least 100, or at least 150 μm in diameter are counted. In some embodiments counting is performed by eye. In some embodiments counting is performed using an automated system, which may be equipped with appropriate image processing software.

In some embodiments a method further comprises isolating an organoid from the composition. In some embodiments a method further comprises isolating an organoid from the composition and transferring the organoid to a different culture vessel. In some embodiments a method further comprises isolating an organoid from the composition and introducing the organoid into a subject. In some embodiments the organoid is introduced in an orthotopic location. In some embodiments the subject is of the same species as the cells. In some embodiments the subject is a non-human animal. In some embodiments the subject is a human. In some embodiments a method further comprises assessing the in vivo development of the organoid. For example, in some embodiments a method further comprises determining whether the organoid gives rise to multiple differentiated cell types. In some embodiments a method further comprises determining whether the organoid gives rise to a substantially complete structure or organ. In some embodiments a method further comprises determining whether the organoid gives rise to a structure or organ that has at least one physiological function of a naturally occurring mature structure or organ. In some embodiments a method comprises dissociating an isolated organoid. In some embodiments cells obtained from the organoid are further cultured.

In some embodiments stem cells are contacted with a test agent prior to being cultured in the composition. In some embodiments cells are contacted with a test agent while being cultured in the composition. In some embodiments the effect of the test agent on organoid formation or phenotype is assessed. In some embodiments a test agent that enhances or inhibits organoid formation is identified. In some embodiments an agent so identified can be used to promote organoid formation. In some embodiments a test agent that alters organoid phenotype is identified. In some embodiments a test agent that does not detectably alter organoid formation or phenotype is identified. In some embodiments a method is used to test an agent to which humans or animals are exposed or may be exposed, e.g., an agent being used or contemplated for use in a food, beverage, supplement, medication, pesticide, herbicide, fertilizer, manufacturing process, or article of manufacture, or produced as a byproduct or waste product in a manufacturing process. In some embodiments a compound that exerts a deleterious effect on organoid formation, maintenance, phenotype, or in vivo development at a concentration relevant to anticipated or actual exposure of human or non-human subject may be identified.

In some aspects, the disclosure provides a variety of kits. In some embodiments, a kit comprises (a) an EMT-inducing agent; and (b) an EMT-cooperating agent, e.g., an agent that comprises, encodes, or induces expression of a polypeptide comprising at least one Sox protein or enhances activity of a polypeptide comprising at least one Sox protein. In some embodiments a kit comprises a first nucleic acid that encodes a polypeptide comprising an EMT-TF and a second nucleic acid that encodes a polypeptide comprising an EMT-cooperating TF, wherein the EMT-TF and the EMT-cooperating TF are capable of cooperating to promote generation of stem cells. In some embodiments a kit comprises a ROCK inhibitor. In some embodiments a kit comprises Matrigel. Any combination of agents may be provided in various embodiments. The agents may be packaged in individual vessels, e.g., tubes, vials. Compatible agents may be packaged together in the same vessel if desired. In some embodiments a kit further comprises one or more reagents (e.g., antibodies, reporter plasmids, probes, primers) useful for detecting expression of one or more markers characteristic of an epithelial cell, mesenchymal cell, or stem cell or useful for assessing production or presence of stem cells. In some embodiments, cells are provided as part of or in conjunction with the kit. Any of the kits can comprise instructions for use, e.g., instructions for generating stem cells from epithelial cells. Articles in a kit may be individually packaged or contained in individual containers, which may be provided together in a larger container such as a cardboard or styrofoam box. In some embodiments one or more reagents or a kit comprising one or more reagents may meet specified manufacturing and/or quality control criteria, e.g., consistency with good manufacturing practices.

In some embodiments a kit comprises Matrigel and instructions for use, wherein the instructions disclose or reference use of about 5% Matrigel for culture of organoids.

In some embodiments a kit comprises a first reagent(s) suitable for use to assess expression of a gene that encodes an EMT-TF; and (b) a first reagent(s) suitable for use to assess expression of a gene that encodes a Sox protein. In some embodiments a reagent suitable for use to assess expression of a gene comprises a reagent that binds to an expression product of the gene. In some embodiments a reagent comprises (i) a probe or primer for detecting, reverse transcribing, and/or amplifying mRNA that encodes an EMT-TF or Sox protein; (ii) an antibody that binds to an EMT-TF or Sox protein (e.g., for use in IHC); (iii) one or more control reagents; (iv) a detection reagent such as a detectably labeled secondary antibody or a substrate; (v) one or more control or reference samples that can be used for comparison purposes or to verify that a procedure for detecting expression is performed appropriately or is giving accurate results. A control reagent can be used for negative or positive control purposes. A control reagent may be, for example, a probe or primer that does not detect or amplify mRNA encoding an EMT-TF or Sox protein or an antibody that does not bind to an EMT-TF or Sox protein. In some embodiments a probe, primer, antibody, or other reagent is attached to a support, e.g., a bead, slide, chip, etc.

### VII. Screening Methods

In some aspects, cells that have been induced to undergo EMT and/or exposed to an EMT-cooperating agent as described herein are used in a variety of different methods for identifying and/or characterizing agents (which may sometimes be referred to as “screening methods”). In some aspects, methods of identifying an EMT-cooperating agent are provided. In some embodiments cells are contacted with an EMT-inducing agent and a test agent. The ability of the test agent to cooperate with the EMT-inducing agent is assessed. For example, in some embodiments the ability of the test agent to confer on the cells one or more additional stem cell properties or expanded (increased) differentiation potential, as compared with the effect of a robust EMT alone and/or as compared with the effect of the agent alone is assessed. If the presence of the test agent results in generation of cells that have one or more additional stem cell properties or expanded differentiation potential as compared with the effect of a robust EMT alone and/or as compared with the effect of the test agent alone, the test agent is identified as an EMT-cooperating agent. In some embodiments cells that have been contacted with an EMT-inducing agent and a test agent are compared with control cells. In some embodiments suitable control cells are cells of the same type that have been contacted with either agent in the same manner as the test cells but in the absence of the other agent. It will be understood that once the effect of a particular agent on cells is established, it would not be necessary to perform a control using that agent in parallel in subsequent uses.

In some embodiments, cells that have been induced to undergo EMT and exposed to an EMT-cooperating agent are used in a screen to identify compounds that target cancer stem cells (CSCs). For example, in some embodiments a method for testing the ability of a compound to inhibit the growth and/or survival of a cancer stem cell, the method comprising (a) contacting one or more test cells with the compound wherein the one or more test cells has undergone an EMT and been exposed to an EMT-cooperating agent; and (b) detecting the level of inhibition of the growth and/or survival of the one or more test cells by the compound. In some embodiments, the test cells are epithelial cells, e.g., transformed epithelial cells. In some embodiments, the methods further include contacting one or more control cells with the compound and detecting the level of inhibition of the growth and/or survival of the one or more control cells by the compound. In some embodiments, the one or more control cells comprise epithelial cell(s) that have not undergone an EMT, e.g., the cells have not been induced to undergo EMT. In some embodiments, the methods comprise: (a) contacting one or more test cells and one or more control cells with a compound, wherein the one or more test cells has undergone an EMT and been exposed to an EMT-cooperating agent, and the one or more control cells has not undergone an EMT; (b) detecting the level of inhibition of the growth and/or survival of the one or more test cells and control cells by the compound; and (c) identifying the compound as a candidate CSC-selective chemotherapeutic agent if the compound has a greater inhibitory effect on the growth and/or survival of the test cells than the control cells.

A wide variety of test agents can be used in the methods. For example, a test agent can be a small molecule, polypeptide, peptide, nucleic acid, oligonucleotide, lipid, carbohydrate, or hybrid molecule. Compounds can be obtained from natural sources or produced synthetically. Compounds can be at least partially pure or may be present in extracts or other types of mixtures. Extracts or fractions thereof can be produced from, e.g., plants, animals, microorganisms, marine organisms, fermentation broths (e.g., soil, bacterial or fungal fermentation broths), etc. In some embodiments, a compound collection (“library”) is tested. The library may comprise, e.g., between 100 and 500,000 compounds, or more. Compounds are often arrayed in multiwell plates. They can be dissolved in a solvent (e.g., DMSO) or provided in dry form, e.g., as a powder or solid. Collections of synthetic, semi-synthetic, and/or naturally occurring compounds can be tested. Compound libraries can comprise structurally related, structurally diverse, or structurally unrelated compounds. Compounds may be artificial (having a structure invented by man and not found in nature) or naturally occurring. In some embodiments, a library comprises at least some compounds that have been identified as “hits” or “leads” in other drug discovery programs and/or derivatives thereof. A compound library can comprise natural products and/or compounds generated using non-directed or directed synthetic organic chemistry. Often a compound library is a small molecule library. Other libraries of interest include peptide or peptoid libraries, cDNA libraries, and oligonucleotide libraries. A library can be focused (e.g., composed primarily of compounds having the same core structure, derived from the same precursor, or having at least one biochemical activity in common).

Compound libraries are available from a number of commercial vendors such as Tocris BioScience, Nanosyn, BioFocus, and from government entities. For example, the Molecular Libraries Small Molecule Repository (MLSMR), a component of the U.S. National Institutes of Health (NIH) Molecular Libraries Program is designed to identify, acquire, maintain, and distribute a collection of >300,000 chemically diverse compounds with known and unknown biological activities for use, e.g., in high-throughput screening (HTS) assays (see https://mli.nih.gov/mli/). The NIH Clinical Collection (NCC) is a plated array of approximately 450 small molecules that have a history of use in human clinical trials. These compounds are highly drug-like with known safety profiles. The NCC collection is arrayed in six 96-well plates. 50 μl of each compound is supplied, as an approximately 10 mM solution in 100% DMSO. In some embodiments, a collection of compounds comprising “approved human drugs” is tested. An “approved human drug” is a compound that has been approved for use in treating humans by a government regulatory agency such as the US Food and Drug Administration, European Medicines Evaluation Agency, or a similar agency responsible for evaluating at least the safety of therapeutic agents prior to allowing them to be marketed. The test agent may be, e.g., an antineoplastic, antibacterial, antiviral, antifungal, antiprotozoal, antiparasitic, antidepressant, antipsychotic, anesthetic, antianginal, antihypertensive, antiarrhythmic, antiinflammatory, analgesic, antithrombotic, antiemetic, immunomodulator, antidiabetic, lipid- or cholesterol-lowering (e.g., statin), anticonvulsant, anticoagulant, antianxiety, hypnotic (sleep-inducing), hormonal, or anti-hormonal drug, etc. In some embodiments, a compound is one that has undergone at least some preclinical or clinical development or has been determined or predicted to have “drug-like” properties. For example, the test agent may have completed a Phase I trial or at least a preclinical study in non-human animals and shown evidence of safety and tolerability. In some embodiments, a test agent is substantially non-toxic to cells of an organism to which the compound may be administered or cells in which the compound may be tested, at the concentration to be used or, in some embodiments, at concentrations up to 10-fold, 100-fold, or 1,000-fold higher than the concentration to be used. For example, there may be no statistically significant effect on cell viability and/or proliferation, or the reduction in viability or proliferation can be no more than 1%, 5%, or 10% in various embodiments. Cytotoxicity and/or effect on cell proliferation can be assessed using any of a variety of assays. Exemplary methods of assessing cell viability and/or proliferation are mentioned above. In some embodiments, at least some cytotoxicity would be acceptable or, in some embodiments, desirable. For example, a compound exhibiting differential cytotoxicity towards CSCs as compared with noncancerous cells and/or exhibiting differential cytotoxicity towards CSCs as compared with cancer cells that are not CSCs or as compared with a bulk population of cancer cells comprising mainly non-CSCs would be of significant interest. In some embodiments, a test agent is not a compound that is found in a cell culture medium known or used in the art, e.g., culture medium suitable for culturing vertebrate, e.g., mammalian cells or, if the test agent is a compound that is found in a cell culture medium known or used in the art, the test agent is used at a different, e.g., higher, concentration when used in a method disclosed herein.

In some embodiments, methods of identifying an agent, e.g., a small molecule, that can substitute for an EMT-TF or EMT-cooperating TF in promoting generation of stem cells are provided. In some embodiments methods of identifying an

In various embodiments of any aspect herein pertaining to screening methods (e.g., methods of identifying agents), the screen may be performed using a single test agent or multiple test agents in a given reaction vessel. In various embodiments the number of reaction vessels and/or test agents is at least 10; 100; 1000; 10,000; 100,000, or more. In some embodiments of any aspect herein pertaining at least in part to screening methods (e.g., methods of identifying agents) a high throughput screen (HTS) is performed. High throughput screens often involve testing large numbers of test agents with high efficiency, e.g., in parallel. For example, tens or hundreds of thousands of agents may be routinely screened in short periods of time, e.g., hours to days. Such screening is often performed in multiwell plates (sometimes referred to as microwell or microtiter plates or microplates) containing, e.g., 96, 384, 1536, 3456, or more wells or other vessels in which multiple physically separated depressions, wells, cavities, or areas (collectively “wells”) are present in or on a substrate. Different test agent(s) may be present in or added to the different wells. It will be understood that some wells may be empty, may comprise replicates, or may contain control agents or vehicle. High throughput screens may involve use of automation, e.g., for liquid handling, imaging, and/or data acquisition or processing, etc. In some embodiments an integrated robot system comprising one or more robots transports assay-microplates from station to station for, e.g., addition, mixing, and/or incubation of assay constituents (e.g., test agent, target, substrate) and, in some embodiments, readout or detection. A HTS system may prepare, incubate, and analyze many plates simultaneously. Certain general principles and techniques that may be applied in embodiments of a HTS are described in Macarrón R & Hertzberg R P. Design and implementation of high-throughput screening assays. Methods Mol Biol., 565:1-32, 2009 and/or An W F & Tolliday N J., Introduction: cell-based assays for high-throughput screening. Methods Mol Biol. 486:1-12, 2009, and/or references in either of these. Exemplary methods are also disclosed in High Throughput Screening: Methods and Protocols (Methods in Molecular Biology) by William P. Janzen (2002) and High-Throughput Screening in Drug Discovery (Methods and Principles in Medicinal Chemistry) (2006) by Jorg H\ddot{υ}ser. Test agent(s) showing an activity of interest (sometimes termed “hits”) may be retested and/or, optionally (e.g., depending at least in part on results of restesting) selected for further testing, development, or use. In some embodiments one or more structural analogs of a hit is synthesized. Such analogs may, for example, comprise substitution of one or more functional groups or heteroatoms present in the hit by a different functional group or heteroatom or substituting a heteroatom or functional group present in place of a hydrogen in the hit, etc. In some embodiments one or more such analog(s) are then tested for a property or activity of interest (e.g., ability to inhibit survival or proliferation of CSCs; ability to cooperate with EMT, ability to substitute for an EMT-TF or EMT-cooperating TF, etc.). In some embodiments one or more analog(s) are tested for, e.g., specificity, selectivity for CSCs versus non-CSCs, solubility, plasma half-life, toxicity to normal cells in vitro, toxicity to a test animal, In some embodiments an analog having an improved or property or activity is identified. An “improved property or activity” is one that makes the analog more suitable or effective for use in an application of interest than the agent with which it is compared (e.g., the original hit). In some embodiments multiple cycles of analog synthesis and testing are performed.

Positive and/or negative controls may be used in any of the screens. An appropriate positive or negative control can be selected based at least in part on the assay. A negative control may be to perform the assay in the absence of a test agent.

In some embodiments, information derived from sequence analysis, mutational analysis, and/or structural analysis is used in the identification of a modulator, e.g., an inhibitor or activator, of an EMT-TF or EMT-cooperating TF or of a protein that functions in a signaling pathway that leads to expression of such a TF. For example, in some embodiments a structure (e.g., a two-dimensional or three-dimensional structure) of a target, e.g., a TF, generated at least in part using, e.g., nuclear magnetic resonance, homology modeling, and/or X-ray crystallography is used. In some embodiments a structure obtained with a ligand (e.g., an inhibitor) bound to the target may be used. In some embodiments a computer-aided computational approach sometimes referred to as “virtual screening” is used in the identification of candidate modulators. Structures of compounds, e.g., small molecules may be screened for ability to bind to a region (e.g., a “pocket”) accessible to the compound. The region may be any region accessible to the compound, e.g., a concave region on the surface or a cleft or a region involved in dimerization. A variety of docking and pharmacophore-based algorithms are known in the art, and computer programs implementing such algorithms are available. Commonly used programs include Gold, Dock, Glide, FlexX, Fred, and LigandFit (including the most recent releases thereof). See, e.g., Ghosh, S., et al., Current Opinion in Chemical Biology, 10(3): 194-2-2, 2006; McInnes C., Current Opinion in Chemical Biology; 11(5): 494-502, 2007, and references in either of the foregoing articles, which are incorporated herein by reference. In some embodiments a virtual screening algorithm may involve two major phases: searching (also called “docking”) and scoring. During the first phase, the program automatically generates a set of candidate complexes of two molecules (test compound and target molecule) and determines the energy of interaction of the candidate complexes. The scoring phase assigns scores to the candidate complexes and selects a structure that displays favorable interactions based at least in part on the energy. To perform virtual screening, this process may be repeated with a large number of test compounds to identify those that, for example, display the most favorable interactions with the target. In some embodiments, low-energy binding modes of a small molecule within an active site or possible active site or other target region are identified. In some embodiments a compound capable of docking at a site where mutations are known to inhibit activity of the target is identified. Variations may include the use of rigid or flexible docking algorithms and/or including the potential binding of water molecules. In some embodiments the three-dimensional structure of an enzyme's active site may be used to identify potential inhibitors. Agent(s) that have the potential to bind in or near an active site may be identified. These predictions may then be tested using the actual compound. A new inhibitor thus identified may then be used to obtain a structure of the enzyme in an inhibitor/enzyme complex to show how the molecule is binding to the active site. Further changes may be made to the inhibitor, e.g., to try to improve binding. This cycle may be repeated until an inhibitor of sufficient predicted or actual potency (e.g., a desired potency for therapeutic purposes) is identified. Numerous small molecule structures are available and can be used for virtual screening. A collection of compound structures may sometimes referred to as a “virtual library”. For example, ZINC is a publicly available database containing structures of millions of commercially available compounds that can be used for virtual screening (http://zinc.docking.org/; Shoichet, J. Chem. Inf. Model., 45(1):177-82, 2005). A database containing about 250,000 small molecule structures is available on the National Cancer Institute (U.S.) website (at http://129.43.27.140/ncidb2/). In some embodiments multiple small molecules may be screened, e.g., up to 50,000; 100,000; 250,000; 500,000, or up to 1 million, 2 million, 5 million, 10 million, or more. Compounds can be scored and, optionally, ranked by their potential to bind to a target. Compounds identified in virtual screens can be tested in cell-free or cell-based assays or in animal models to confirm their ability to inhibit activity of a target molecule and/or to assess their biological and/or pharmacological activity. Computational approaches may be used to predict one or more physico-chemical, pharmacokinetic and/or pharmacodynamic properties of compounds identified in a physical or virtual screen. Such information may be used, e.g., to select one or more hits for, e.g., further testing, development, or use. For example, small molecules having characteristics typical of “drug-like” molecules may be selected and/or small molecules having one or more undesired characteristics may be avoided.

In some aspects of any screening and/or characterization methods, test agents are contacted with test cells (and optionally control cells) or used in cell-free assays at a predetermined concentration. In some embodiment the concentration is about up to 1 nM. In some embodiments the concentration is between about 1 nM and about 100 nM. In some embodiments the concentration is between about 100 nM and about 10 μM. In some embodiments the concentration is at or above 10 μM, e.g., between 10 μM and 100 μM. Following incubation for an appropriate time, optionally a predetermined time, the effect of compounds or composition on a parameter of interest in the test cells is determined by an appropriate method known to one of ordinary skill in the art, e.g., as described herein. Cells can be contacted with compounds for various periods of time. In certain embodiments cells are contacted for between 12 hours and 20 days, e.g., for between 1 and 10 days, for between 2 and 5 days, or any intervening range or particular value. Cells can be contacted transiently or continuously. If desired, the compound can be removed prior to assessing the effect on the cells.

### VIII. Methods of Classification, Cancer Prognosis, and Treatment Selection

In some aspects, the disclosure encompasses the recognition that high levels of expression and/or activity of an EMT-TF and a TF that cooperates with that EMT-TF in tumors is associated with poor patient survival, consistent with promotion of tumor-initiating and metastatic ability by the cooperation of such TFs. In some aspects, the disclosure provides methods of classifying a cell, sample, tumor, or subject. In some embodiments the methods comprise classifying a cell, sample, tumor, or subject based on the expression and/or activity of at least two genes, wherein the first gene encodes an EMT-TF, e.g., Slug, and the second gene encodes an EMT-cooperating TF, e.g., a Sox protein. In some embodiments, the level of expression of a gene is assessed by determining the level of an expression product of the gene in the sample. In some embodiments an expression product is RNA, e.g., mRNA. In some embodiments an expression product is a polypeptide. In some embodiments expression of a gene that is regulated (e.g., upregulated) by an EMT-TF is used as a surrogate for expression of the EMT-TF. In some embodiments expression of a gene that is regulated (e.g., upregulated) by an EMT-cooperating TF is used as a measure of overall activity of the EMT-cooperating TF. In some embodiments expression of the surrogate gene (i.e., the gene regulated by the TF) is assessed instead of or in addition to assessing expression of the TF. One of ordinary skill in the art will understand that expression of a useful surrogate gene should generally correlate closely with expression and/or overall activity of the TF for which it serves as a surrogate.

In some embodiments a method for classifying a cell, sample, tumor, comprises assessing the level of an RNA that encodes an EMT-TF and the level of an RNA that encodes a Sox protein in the cell, sample, or tumor. In some embodiments a method for classifying a sample comprises assessing the level of an EMT TF and the level of a Sox protein in the cell, sample, or tumor. In some embodiments, a method of classifying a cell comprises steps of: (a) providing a cell; and (b) assessing expression of a first gene that encodes an EMT-TF and a second gene that encodes a Sox protein, wherein increased level of expression of the first and second genes is correlated with a phenotypic characteristic, thereby classifying the cell with respect to the phenotypic characteristic. In some embodiments the cell is a tumor cell, and the phenotypic characteristic is propensity of the tumor cell to metastasize. In some embodiments the cell is a tumor cell, and the phenotypic characteristic is tumor-initiating capacity of the cell. In some embodiments the cell is obtained from a tumor. In some embodiments, a method of classifying a tumor cell comprises steps of: (a) providing a sample; and (b) assessing expression of a first gene that encodes or is regulated by an EMT-TF and a second gene that encodes or is regulated by a Sox protein, wherein increased level of expression of the first and second genes indicates that the tumor cell has a propensity to metastasize. In some embodiments, a method of classifying a tumor cell comprises steps of: (a) providing a sample; and (b) assessing expression of a first gene that encodes or is regulated by an EMT-TF and a second gene that encodes or is regulated by a Sox protein, wherein increased level of expression of the first and second genes indicates that the tumor cell has increased tumor-initiating capacity. In some embodiments the cell is a normal cell, and the phenotypic characteristic is a SC trait. In some embodiments the phenotypic characteristic is multi-lineage potential. In some embodiments the cell is a normal cell, and the phenotypic characteristic is a SC trait. In some embodiments the phenotypic characteristic is multi-lineage potential. In some embodiments, a method of classifying a cell comprises steps of: (a) providing a cell; and (b) assessing expression of a first gene that encodes or is regulated by an EMT-TF and a second gene that encodes or is regulated by a Sox protein, wherein increased level of expression of the first and second genes indicates that the cell has multi-lineage potential.

In some embodiments, a method of identifying a tumor cell that has tumor-initiating capacity comprises steps of: (a) providing a sample comprising at least one tumor cell; and (b) assessing expression of a first gene that encodes or is regulated by an EMT-TF and a second gene that encodes or is regulated by a Sox protein in at least one tumor cell of the sample; and (c) identifying a cell that has increased expression of the first and second genes, thereby identifying a cell that has multi-lineage potential. In some embodiments, a method of identifying a tumor cell that has propensity to metastasize comprises steps of: (a) providing a sample comprising at least one tumor cell; and (b) assessing expression of a first gene that encodes or is regulated by an EMT-TF and a second gene that encodes or is regulated by a Sox protein in at least one tumor cell of the sample; and (c) identifying a cell that has increased expression of the first and second genes, thereby identifying a cell that has propensity to metastasize. In some embodiments the sample comprises multiple cells, and the method further comprises separating at least one cell that has increased expression of the first and second genes from at least one cell that does not have increased expression of both of the genes.

In some embodiments, a method of classifying a sample comprises steps of: (a) providing a sample; and (b) assessing expression of a first gene that encodes or is regulated by an EMT-TF and a second gene that encodes or is regulated a Sox protein, wherein increased level of expression of the first and second genes is correlated with a phenotypic characteristic, thereby classifying the sample with respect to the phenotypic characteristic. In some embodiments the sample is derived from or comprises tumor cells, and the phenotypic characteristic is propensity to metastasize. In some embodiments the sample is derived from or comprises tumor cells, and the phenotypic characteristic is tumor-initiating capacity. In some embodiments the sample is obtained from a subject in need of monitoring or treatment for a tumor. In some embodiments, a method comprises steps of: (a) providing a sample comprising one or more cells; and (b) assessing expression of a first gene that encodes or is regulated by an EMT-TF and a second gene that encodes or is regulated by a Sox protein in at least some of the cells. In some embodiments the method comprises determining the proportion of cells that have increased expression of both the first and second genes.

In some embodiments a method of classifying a tumor comprises: (a) determining the expression level of a first gene that encodes or is regulated by an EMT-TF and a second gene that encodes or is regulated by a Sox protein in one or more samples obtained from a tumor; (b) comparing the expression level of the first and the second genes with control expression levels of said genes; and (c) classifying the tumor with respect to cancer prognosis, wherein a greater level of expression of both the first and the second genes in the sample(s) obtained from the tumor as compared with the respective control levels is indicative of an increased likelihood of poor outcome. In some embodiments a method of classifying a tumor comprises: (a) determining the level of expression of a first gene that encodes or is regulated by an EMT-TF and a second gene that encodes or is regulated by a Sox protein in one or more samples obtained from a tumor; (b) comparing the level of expression of the first and the second genes with control expression levels of said genes; and (c) classifying the tumor with respect to cancer prognosis, wherein a greater level of expression of both first and second genes in the sample(s) obtained from the tumor as compared with the respective control levels is indicative that the sample originates from a tumor or subject that falls within a poor prognosis subclass. In some embodiments expression of the first and second genes is assessed in the same sample. In some embodiments expression of the first and second genes is assessed in different samples. Typically, if expression of the first and second genes is assessed in the same sample, the expression level of both genes must be increased in the sample in order for the tumor to be considered to have increased expression of the first and second genes. Various risk categories may be defined. For example, tumors may be classified as at low, intermediate, or high risk of poor outcome. Samples may be classified as arising from tumors at low, intermediate, or high risk of poor outcome. A variety of statistical methods may be used to correlate the risk of poor outcome with the relative or absolute expression levels of the first and second genes.

In some embodiments a method comprises assigning a score to a sample or tumor based on expression level of a first gene that encodes or is regulated by an EMT-TF and a second gene that encodes or is regulated by a Sox protein. In some embodiments a score is based at least in part on the proportion of cells that have increased expression of both the first and second genes. In some embodiments a score is based at least in part on the expression levels of both the first and second genes. In some embodiments a score is based at least in part on the expression levels of both the first and second genes and the proportion of cells that have increased expression of both first and second genes. In some embodiments a sample is scored as positive if it contains ≧5% positive cells and negative if it contains less than 5% positive cells. In some embodiments a positive sample is scored 1 for weak expression, 2 for moderate expression and 3 for strong expression. A higher score in this system indicates a less favorable prognosis than a lower score, e.g., more likely occurrence of metastasis, shorter disease free survival, lower likelihood of 5 year survival, lower likelihood of 10 year survival, or shorter average survival. In some embodiments samples or tumors that exhibit at least moderate expression of both the first and second genes are classified as having a poor prognosis or increased likelihood of poor outcome as compared with samples or tumors that do not exhibit at least moderate expression of both the first and second genes. In some embodiments a scoring system useful to assign tumors to a prognostic category based on expression of a first gene that encodes or is regulated by an EMT-TF and a second gene that encodes or is regulated by a Sox protein can be established based on a panel of tumors with known outcomes. In some embodiments a score is assigned giving equal or approximately weight to expression of the first and second genes. In some embodiments a score is assigned giving different weights to the first and second genes. In some embodiments the genes are assigned weights that are within a factor of up to 3-fold of each other. A score can be obtained by evaluating one field or multiple fields in a cell or tissue sample. Multiple samples from a tumor may be evaluated in some embodiments. It will be understood that “no detectable expression” could mean that the level detected, if any, is not noticeably or not significantly different to background levels. In some embodiments, at least 20, 50, 100, 200, 300, 400, 500, 1000 cells, or more (e.g., tumor cells) are assessed to evaluate expression in a sample or tumor, e.g., to assign a score to a sample or tumor. It will be appreciated that a score can be represented using numbers or using any suitable set of symbols, words, and/or numbers.

The number of categories in a useful scoring or classification system may be at least 2, e.g., between 2 and 10. A scoring or classification system is often effective to divide a population of tumors or subjects into groups that differ in terms of an outcome such as local progression, local recurrence, discovery or progression of regional or distant metastasis, death from any cause, or death directly attributable to cancer. An outcome may be assessed over a given time period, e.g., 2 years, 5 years, 10 years, 15 years, or 20 years from a relevant date, e.g., the date of diagnosis or approximate date of diagnosis (e.g., within about 1 month of diagnosis) or a date after diagnosis, e.g., a date of initiating treatment. Methods and criteria for evaluating progression, response to treatment, existence of metastases, and other outcomes are known in the art and may include objective measurements (e.g., anatomical tumor burden) and criteria, clinical evaluation of symptoms, or combinations thereof. For example, 1, 2, or 3-dimensional imaging (e.g., using X-ray, CT scan, or MRI scan, etc.) and/or functional imaging may be used to detect or assess lesions (local or metastatic), e.g., to assess number and dimensions of lesions, detect new lesions, etc. In some embodiments, a difference between groups is statistically significant as determined using an appropriate statistical test or analysis method, which can be selected by one of ordinary skill in the art. In some embodiments a difference between groups would be considered clinically meaningful by one of ordinary skill in the art.

In some embodiments, results of assessing expression of a first gene that encodes or is regulated by an EMT-TF and a second gene that encodes or is regulated by a Sox protein are of use in selecting an appropriate treatment regimen for a subject in need of treatment of a tumor and/or selecting the type or frequency of procedures to be used to monitor a subject for local or metastatic recurrence after therapy and/or the frequency with which such procedures are performed. For example, subjects classified as having a poor prognosis (being at high risk of poor outcome) may be treated and/or monitored more intensively than those classified as having a good prognosis. Thus a method can further comprise using information obtained from assessment of expression of a first gene that encodes or is regulated an EMT-TF and a second gene that encodes a Sox protein to help in selecting a treatment or monitoring regimen for a subject suffering from cancer or at increased risk of cancer or at risk of cancer recurrence or in providing an estimate of the risk of poor outcome such as cancer related mortality or recurrence. The information may be used, for example, by a subject's health care provider in selecting a treatment or in treating a subject. A health care provider could also or alternatively use the information to provide a cancer patient with an accurate assessment of his or her prognosis. In some embodiments, a method comprises making a treatment selection or administering a treatment based at least in part on the result of an assessment of expression of a first gene that encodes or is regulated an EMT-TF and a second gene that encodes or is regulated by a Sox protein. In some embodiments, a method comprises selecting or administering more aggressive treatment or treatment regimen to a subject, if the subject is determined to have a poor prognosis. In some embodiments, a method comprises selecting or administering more aggressive treatment or treatment regimen if the subject is determined to have a tumor that exhibits increased expression of a first gene that encodes or is regulated an EMT-TF and a second gene that encodes or is regulated by a Sox protein. A “treatment regimen” refers to a course of treatment involving administration of an agent or use of a non-pharmacological therapy multiple times over a period of time, e.g., over weeks or months. A treatment regimen can include one or more pharmacological agents (often referred to as “drugs” or “compounds”) and/or one or more non-pharmacological therapies such as radiation, surgery, etc. A treatment regimen can include the identity of agents to be administered to a subject and may include details such as the dose(s), dosing interval(s), number of courses, route of administration, etc. “Monitoring regimen” refers to repeated evaluation of a subject over time by a health care provider, typically separated in time by weeks, months, or years. The repeated evaluations can be on a regular or predetermined approximate schedule and are often performed to determine whether a cancer has recurred or to track the effect of a treatment on a tumor or subject.

Treatments and treatment regimens that are considered “more aggressive” or “less aggressive” for treatment of particular tumor types are known in the art. In various embodiments “more aggressive” treatment (also referred to as “intensive” or “more intensive” treatment herein) can comprise, for example, (i) administration of a dose of one or more agents (e.g., chemotherapeutic agent) that is at the higher end of the acceptable dosage range (e.g., a high dose rather than a medium or low dose, or a medium dose rather than a low dose) and/or administration of a number of doses or a number of courses at the higher end of the acceptable range and/or use of non-hormonal cytotoxic/cytostatic chemotherapy; (ii) administration of multiple agents rather than a single agent; (iii) administration of more, or more intense, radiation treatments; (iv) administration of a greater number of agents in a combination therapy; (v) use of adjuvant therapy; (vi) more extensive surgery, such as removing an organ rather than organ-conserving therapy (e.g., mastectomy rather than breast-conserving surgery such as lumpectomy). For example, in some embodiments a method comprises (i) selecting that the subject not receive chemotherapy (e.g., adjuvant chemotherapy) if the tumor is considered to have a good prognosis; or (ii) selecting that the subject receive chemotherapy (e.g., adjuvant chemotherapy), or administering such chemotherapy, if the tumor is considered to have a poor prognosis. In some embodiments, a method comprises selecting that a subject receives less aggressive treatment or administering such treatment if the subject is determined to have a good prognosis. In various embodiments “less aggressive” (also referred to as “less intensive”) treatment can comprise, for example, using a dose level or dose number at the lower end of the acceptable range, not administering adjuvant therapy, selecting an organ-conserving therapy rather than removing an organ (e.g., breast-conserving therapy rather than mastectomy), selecting hormonal therapy rather than non-hormonal cytotoxic/cytostatic chemotherapy, or simply monitoring the subject. In some embodiments “more intensive” or “intensive” monitoring comprises, for example, more frequent clinical and/or imaging examination of the subject or use of a more sensitive imaging technique rather than a less sensitive technique.

In some embodiments a method of selecting a monitoring regimen for a subject comprises: (a) providing a subject in need of monitoring for a tumor; (b) obtaining a classification of the tumor based at least in part on the expression level of a first gene that encodes or is regulated by an EMT-TF and a second gene that encodes or is regulated by a Sox protein in one or more samples obtained from the tumor; and (c) selecting a monitoring regimen based at least in part on the classification. In some embodiments a method of monitoring a subject comprises: (a) providing a subject in need of monitoring of a tumor; (b) obtaining a classification of the tumor based at least in part on the expression level of a first gene that encodes or is regulated by an EMT-TF and a second gene that encodes or is regulated by a Sox protein in one or more samples obtained from the tumor; and (c) monitoring the subject using a monitoring regimen selected based at least in part on the classification. In some embodiments a method of monitoring a subject comprises: (a) providing a subject in need of treatment for a tumor; (b) obtaining a measurement of the expression level of a first gene that encodes an EMT-TF and a second gene that encodes or is regulated by a Sox protein in one or more samples obtained from the tumor; and (c) monitoring the subject based at least in part on the result of step (b).

In some embodiments a method of selecting a treatment for a subject comprises: (a) providing a subject in need of treatment for a tumor; (b) obtaining a classification of the tumor based at least in part on the expression level of a first gene that encodes or is regulated by an EMT-TF and a second gene that encodes or is regulated by a Sox protein in one or more samples obtained from the tumor; and (c) selecting a treatment based at least in part on the classification. In some embodiments a method of treating a subject comprises: (a) providing a subject in need of treatment for a tumor; (b) obtaining a measurement of the expression level of a first gene that encodes or is regulated by an EMT-TF and a second gene that encodes or is regulated by a Sox protein in one or more samples obtained from the tumor; and (c) treating the subject based at least in part on the result of step (b). For example, if the tumor exhibits increased expression of both the first and second genes then a more aggressive treatment or treatment regimen may be selected than if the tumor does not exhibit increased expression of both the first and second genes.

In some embodiments, an expression level, e.g., the level of an expression product of a gene, is determined to be “increased” or “not increased” by comparison with a suitable control level or reference level. The terms “reference level” and “control level” may be used interchangeably herein. A suitable control level can be a level that represents a normal level of gene product e.g., a level of gene product existing in cells or tissue in a non-diseased condition and in the absence of conditions that would induce EMT or induce development or proliferation of stem cells. In some embodiments ay method that includes a step of (a) assessing (determining) the expression level of a gene can comprise a step of (b) comparing the expression level with a control level, wherein if the level determined in (a) is greater than the control level, then the level determined in (a) is considered to be “increased” (or, if the level determined in (a) is not greater than the control level, then the level determined in (a) is considered to be “not increased”. For example, in some embodiments if a tumor has an increased expression level of a first gene that encodes or is regulated by an EMT-TF and a second gene that encodes or is regulated by a Sox protein as compared to a control level, the tumor is classified as having a high risk of poor outcome, while if the tumor does not have an increased expression level of a first gene that encodes or is regulated by an EMT-TF and a second gene that encodes or is regulated by a Sox protein relative to a control level, the tumor is classified as having a low risk of poor outcome. A comparison can be performed in various ways. For example, in some embodiments one or more samples are obtained from a tumor, and one or more samples are obtained from nearby normal (non-tumor) tissue composed of similar cell types from the same patient. The relative level of gene products in the tumor sample(s) versus the non-tumor sample(s) is determined. In some embodiments, if the relative level (ratio) of gene products in the tumor samples versus the non-tumor sample(s) is greater than a predetermined value (indicating that cells of the tumor have increased expression), the tumor is classified as high risk. A control level can be a historical measurement. For example, once expression levels in normal tissue and/or tumor tissue from tumors of different outcome categories are established, such levels may be used as a basis for future comparisons. It will be understood that in at least some embodiments a value may be semi-quantitative, qualitative or approximate. For example, in some embodiments visual inspection (e.g., using light microscopy) of a FISH or IHC sample suffices to provide an assessment of expression level without necessarily counting cells or precisely quantifying the intensity of staining. It will also be understood that the details of a scoring system may vary among different tumor types, e.g., tumors arising in different tissues that normally contain different numbers of stem cells.

For purposes of description herein it is generally assumed herein that the control or reference level in a method of classifying a tumor or tumor sample represents normal levels of expression present in non-cancer cells and tissues. However, it will be understood that expression levels characteristic of cancer having a poor outcome could be used as a reference or control level. In that case, expression at a level comparable to, e.g., approximately the same, as or greater than the control level would be indicative of poor cancer prognosis, more aggressive cancer phenotype, or to identify a subject who is a suitable candidate for aggressive treatment or monitoring, while a decreased level of expression as compared with the control level would be predictive of good cancer prognosis, less aggressive cancer phenotype or to identify a subject who does not require or may not benefit from aggressive treatment or monitoring.

Certain methods are stated herein mainly in terms of classifications, conclusions, predictions that can be made if increased expression of an EMT-TF and an EMT-cooperating TF is present. Such methods could equally be stated in terms of conclusions, predictions, or classifications that can be made if increased expression of either or both genes is not present. For example, in some embodiments, if increased expression of an EMT-TF and an EMT-cooperating TF is absent in a sample obtained from a tumor, the tumor is not classified as having a poor prognosis based on the result.

In some embodiments, assessing expression or activity of an EMT-TF and an EMT-cooperating TF in a cell, sample, or tumor is be used together with levels of one or more other (e.g., up to 10) other mRNAs or proteins that are selected for their utility for classification for diagnostic, prognostic, or treatment selection purposes in one or more types of cancer. In certain embodiments expression of an EMT-TF and an EMT-cooperating TF is not measured or analyzed merely as a contributor to a cluster analysis, dendrogram, or heatmap based on gene expression profiling in which expression at least 20; 50; 100; 500; 1,000, or more genes is assessed. In certain embodiments, if expression of an EMT-TF and an EMT-cooperating TF is measured as part of such a gene expression profile, the level of EMT-TF and an EMT-cooperating TF is used to classify samples or tumors (e.g., for diagnostic, prognostic, or treatment selection purposes) in a manner that is distinct from the manner in which the expression of many or most other genes in the gene expression profile are used. For example, the expression level of the EMT-TF and an EMT-cooperating TF may be used independently of most or all of the other measured expression levels or may be weighted more strongly than many or most other mRNAs or proteins in analyzing or using the results.

In some embodiments, assessing expression or activity of an EMT-TF and an EMT-cooperating TF in a cell, sample, or tumor is be used together with any useful classification, prognostic, or treatment selection approach known in the art. For example, in the context of breast cancer, expression or activity of an EMT-TF and an EMT-cooperating TF may be used together with assessment of estrogen receptor, progesterone receptor, and/or HER2/Neu status, or standard methods of grading or staging the cancer, such as the TNM system.

### IX. Methods of Treatment and Compositions of Use Therefor

In some aspects, methods of treating a subject are provided. In some aspects compositions, e.g., pharmaceutical compositions, suitable for performing the methods, are also provided. Certain methods involve inhibiting EMT and inhibiting expression or activity of an EMT-cooperating TF in a subject in need thereof. Certain methods involve cell therapy using stem cells or progenitor cells or cells differentiated therefrom, wherein the stem or progenitor cells are generated by inducing an EMT and increasing expression or activity of an EMT-cooperating TF as described herein, and are then introduced into a subject in need thereof.

Cell-based therapies in which stem or progenitor cells generated as described herein may be employed include embodiments directed to the treatment of a wide variety of diseases and conditions. Examples include neurological diseases or other conditions affecting the nervous system such as Parkinson's disease, Alzheimer's disease, spinal cord injury, traumatic brain injury, peripheral nerve injuries or disease, and stroke. Traumatic injuries (e.g., tissue injuries, fractures), burns, heart disease (e.g., cardiomyopathy due to any of a variety of different causes), diabetes (e.g., type I diabetes involving loss of insulin-producing beta cells), hair loss (alopecia, baldness), vision loss and blindness, tooth loss, osteoarthritis, tendon and ligament damage, osteochondrosis, and muscular dystrophy are other conditions that may benefit through cell-based therapies. Bone, muscle (e.g., cardiac, skeletal, smooth muscle), skin, cartilage, nerve, and brain are among the cells and tissues toward which cell-based therapies can be directed. In some embodiments stem or progenitor cells for hair follicles are generated. In some embodiments such hair follicle stem or progenitor cells or agents capable of promoting generation of such cells are used to treat a subject in need of treatment for hair loss or hair sparseness. In some embodiments stem or progenitor cells for intestine are generated. In some embodiments such cells or agents capable of promoting generation of such cells are used to treat a subject in need of repair of a tissue that is ordinarily characterized by ongoing cell proliferation, e.g., a subject who has been treated with a chemotherapeutic agent that is toxic to such cells. In some embodiments such cells are used to treat a subject in need of repair of the intestinal epithelium, e.g., a subject who has been treated with a chemotherapeutic agent that is toxic to intestinal cells. In some embodiments cells are obtained from a subject. Stem or progenitor cells are generated or expanded ex vivo using a method disclosed herein. Such cells or differentiated progeny thereof are subsequently administered to the subject. In some embodiments cells are obtained from the subject prior to administration of a cytotoxic agent and/or prior to radiation therapy, and the cells prepared ex vivo are administered to the subject during or after a course of therapy with the cytotoxic agent and/or radiation therapy. In general, cells can be administered by any suitable method, such as injection, implantation, etc.

In some embodiments stem cells, progenitor cells or differentiated progeny may be implanted into failing organs (e.g., the heart) to augment function. In some embodiments, stem cells, progenitor cells, or differentiated progeny, may be used to aid in reconstruction or sealing tissues in the context of orthopedic, urologic, gynecologic, plastic, colorectal, and/or oto-laryngological surgeries, hernia repair, etc. Moreover it is envisioned that stem cells, progenitor cells and/or differentiated progeny thereof may be used in the ex vivo and/or in vivo construction or augmentation of tissues or organs such as skin, soft tissues, breast, blood vessels, hair follicles, kidney, liver, pancreas, bladder, etc. In certain embodiments cells may, if desired, be combined with appropriate scaffolds or matrices comprising naturally occurring and/or synthetic materials such as biocompatible, optionally biodegradable, polymers, polypeptides, etc. In some embodiments, where stem or progenitor cells are introduced into the subject, substances may be administered to promote the survival and/or differentiation of such cells in vivo. In some embodiments, where differentiated progeny of stem or progenitor cells are introduced into the subject, substances may be administered to promote the survival of such cells in vivo. For example, one or more growth factors, extracellular matrix components, or cells capable of secreting such substances may be co-administered. In some embodiments, epithelial cells to be used to derive cells for use in cell therapy are obtained from the subject who is the intended recipient. In some embodiments, the epithelial cells are obtained from a different individual, typically a member of the same species. In some embodiments, if desired, cells can be modified to improve their histocompability and/or compatible donors can be selected.

One of ordinary skill in the art would be aware of agents and methods useful to differentiate stem or progenitor cells towards a desired cell lineage or cell type of interest. Desired cell types may be separated from other cells using methods such as cell sorting, binding to resins or matrices, etc. Such separation may be based, e.g., on expression of markers characteristic of the cell type(s) of interest or lack of expression of markers not characteristic of such cell type(s), cell size, light scattering, or other properties.

In some embodiments SCs or progenitor cells spontaneously give rise to more differentiated cell types at a useful level in culture, while continuing to self-renew. Desired cell types can be harvested periodically. In some embodiments SCs or progenitor cells are induced to differentiate by inhibiting expression or activity of an EMT-TF or otherwise reversing EMT and/or by inhibiting expression or activity of an EMT-cooperating TF. In some embodiments such inhibition is achieved using siRNA or antisense oligonucleotides or other methods that do not entail genetic modification such as the use of appropriate small molecules.

In some embodiments SCs, progenitor cells, or cells derived therefrom (e.g., differentiated cells) are introduced into a subject and serve as a cellular vehicle for protein-replacement therapy, e.g., production in vivo of proteins that are lacking in the subject or from which the subject would benefit. For example, in some embodiments such cells produce one or more hormones (e.g., insulin), enzymes, etc. In some embodiments the cells naturally produce the protein(s) of interest. In some embodiments the cells are genetically engineered to produce the protein(s) of interest. In some embodiments the subject suffers from a disease that results in a deficiency or suboptimal level of the protein. In some embodiments the disease is an inherited genetic disease. Many such diseases are known. A compendium of numerous inherited disorders that occur in humans, many of which involve a deficiency in one or more proteins (e.g., due to a mutation in the gene encoding such protein) is provided in McKusick V. A. (1998) Mendelian Inheritance in Man. A Catalog of Human Genes and Genetic Disorders, 12th Edn. The Johns Hopkins University Press, Baltimore, Md. and its online updated version Online Mendelian Inheritance in Man (OMIM), available at the National Center for Biotechnology Information (NCBI) website at http://www.ncbi.nlm.nih.gov/omim. A compendium of numerous inherited disorders that occur in various non-human animals is provided in Online Mendelian Inheritance in Animals (OMIA) Reprogen, Faculty of Veterinary Science, University of Sydney, available on the World Wide Web at URL: http://omia.angis.org.au/ or on the NCBI website at http://www.ncbi.nlm.nih.gov/omia. Examples of diseases or conditions in which cell-based protein replacement therapy is of interest include, e.g., diabetes (e.g., Type I diabetes), hemophilia (resulting from clotting factor deficiency), adenosine deaminase deficiency, anemia (e.g., resulting at least in part from relative deficiency of erythropoietin from any of various causes), pancreatic enzyme deficiency (e.g., resulting from chronic pancreatitis or other injury to the pancreas or cystic fibrosis), immunodeficiencies, alpha-1 anti-trypsin deficiency, Wilson's disease, to name but a few. In some embodiments the protein is one that is normally present or secreted into the blood and, in some embodiments, acts on cells or substances at a remote location. In some embodiments the protein is one that is normally primarily localized to or active in a particular organ or tissue such as the liver. In some embodiments the protein is a transmembrane transporter or a protein normally acts intracellularly. In such instances, providing the subject with a population of cells that express the transporter or protein may at least in part ameliorate the deficiency. For example, in some embodiments a population of liver or intestinal or lung cells that express transporter or metabolic enzyme(s) that are deficient in a subject are provided. The transporter or enzyme may take up, process, or metabolize a potentially injurious substance. In some embodiments the introduced cells are autologous to the subject. In some embodiments such cells have had a genetic defect repaired ex vivo prior to being introduced. In some embodiments a chromosomal sequence harboring a mutation is changed to afunctional sequence, e.g., a normal sequence. Precise alterations can be accomplished using methods known in the art such as homologous recombination. In some embodiments a functional sequence, e.g., a normal sequence, is inserted at a site distinct from the endogenous locus corresponding to the inserted sequence. In some embodiments the cells are introduced into an organ or tissue corresponding to that which normally produces the protein or corresponding to that from which the original cells were obtained (e.g., cells derived from the liver are introduced into the liver, etc.).

In some embodiments agents are administered to a subject in order to induce generation of stem or progenitor cells in vivo. In some embodiments at least some stem or progenitor cells are also introduced. In some embodiments stem or progenitor cells are not introduced. In some embodiments, two or more agents are administered in combination. In some embodiments “in combination”, as used herein, with regard to combination treatment means with respect to administration of first and second agents, administration performed such that (i) a dose of the second compound is administered before more than 90% of the most recently administered dose of the first agent has been metabolized to an inactive form or excreted from the body; or (ii) doses of the first and second compound are administered within 48 hours of each other, or (iii) the agents are administered during overlapping time periods (e.g., by continuous or intermittent infusion); or (iv) any combination of the foregoing. Multiple agents are considered to be administered in combination if the afore-mentioned criteria are met with respect to all agents, or in some embodiments, if each agent can be considered a “second agent” with respect to at least one other compound of the combination. The agents may, but need not be, administered together as components of a single composition. In some embodiments, they may be administered individually at substantially the same time (by which is meant within less than 10 minutes of one another). In some embodiments they may be administered individually within a short time of one another (by which is meant less than 3 hours, sometimes less than 1 hour, apart). The agents may, but need not, be administered by the same route of administration. Administration of multiple agents in any order is encompassed. One or more of the agents may be administered multiple times. Administration may be performed multiple times at varying or regular intervals, for days, weeks, months, years, or indefinitely in various embodiments.

In some embodiments of any aspect herein pertaining to administration of agents, first and second agents are administered within 2, 4, 8, 12, 24, or 48 hours of each other at least once, wherein a first agent modulates an endogenous EMT-inducing molecule, e.g., an EMT-TF, and a second agent modulates an endogenous molecule that cooperates with EMT, e.g., an EMT-cooperating TF. In some embodiments, first and second agents are administered within 3, 4, 5, 6, or 7 days of each other at least once, wherein a first agent modulates an endogenous EMT-inducing molecule, e.g., an EMT-TF, and a second agent modulates an endogenous molecule that cooperates with EMT, e.g., an EMT-cooperating TF.

Generation of stem or progenitor cells in vivo could be of benefit in various diseases and conditions, e.g., those conditions mentioned above for which cell-based therapy is of use. For example, generation of stem or progenitor cells in vivo may provide an increased number of cells for repair of a damaged or defective organ or tissue. To that end, agents may be administered locally, at or near a site of tissue or organ damage or defect.

In another aspect, methods of inhibiting one or more endogenous EMT-inducing agents and inhibiting one or more endogenous EMT-cooperating agents in a subject in need thereof are provided. In some embodiments, a method comprises administering a first agent that inhibits an endogenous EMT-inducing agent and a second agent that inhibits an endogenous EMT-cooperating agent to the subject. In some embodiments the subject is at risk of or suffering from a condition in which excessive formation of stem cells or progenitor cells or excessive proliferation of stem cells or progenitor cells occurs in vivo and contributes to one or more pathologic features of the condition. In certain embodiments a method comprises administering a first agent that inhibits EMT and a second agent that inhibits an EMT-cooperating TF. For example, in some embodiments a method comprises administering a first agent that inhibits EMT and a second agent that inhibits a SoxE protein, e.g., Sox9, Sox10, or both.

In some embodiments a subject is in need of treatment for cancer. As known in the art, cancer is a disease characterized by uncontrolled or aberrantly controlled cell proliferation and other malignant cellular properties. The EMT process allows cells to acquire migratory properties, which facilitate cancer cell dissemination and metastasis. In addition, cancer cells that have undergone EMT exhibit increased self-renewal capacity and tumor-initiating capacity, properties characteristic of cancer stem cells. As described herein, certain genetic pathways or processes can cooperate with the EMT to, e.g., promote such properties. In some embodiments inhibiting one or more endogenous agent capable of inducing and/or maintaining EMT and inhibiting one or more endogenous agents that cooperate with EMT (e.g., endogenous EMT-cooperating TFs such as Sox9) reduces tumor progression (e.g., tumor metastasis) and/or tumor relapse or recurrence. In some embodiments inhibiting of endogenous molecules capable of inducing and/or maintaining EMT and inhibiting one or more endogenous agents that cooperate with EMT reduces resistance to therapy (e.g., reduces resistance to one or more standard chemotherapeutic agents or radiation) and/or renders cancer cells more susceptible to endogenous immune-mediated defense mechanisms. In particular embodiments, a first agent that inhibits Slug and a second agent that inhibits Sox9 and/or Sox10 are administered.

In some embodiments, the cancer is also treated using chemotherapy, radiation, and/or surgery. In some embodiments inhibitor(s) are administered locally, e.g., at the site of a tumor, e.g., prior to, during, and/or following surgery or radiation. In some embodiments, agents are administered in the vicinity of the tumor, or at a site where a tumor has been or will be surgically removed or irradiated. For example, in some non-limiting embodiments, agents are administered at least once within the 4 weeks preceding surgery and/or at least once within the 4 weeks following surgery. In some non-limiting embodiments, agents may be administered at least once within the 4 weeks preceding initiation of a course of radiation treatments and/or at least once within the 4 weeks following completion of a course of radiation treatments, and optionally one or more times between radiation treatments. In some embodiment agents are administered prior to or following such time intervals instead or additionally.

As used herein, the term cancer includes, but is not limited to, the following types of cancer: breast cancer; biliary tract cancer; bladder cancer; brain cancer including glioblastomas and medulloblastomas; cervical cancer; choriocarcinoma; colon cancer; endometrial cancer; esophageal cancer; gastric cancer; hematological neoplasms including acute lymphocytic and myelogenous leukemia; T-cell acute lymphoblastic leukemia/lymphoma; hairy cell leukemia; chronic myelogenous leukemia, multiple myeloma; AIDS-associated leukemias and adult T-cell leukemia/lymphoma; intraepithelial neoplasms including Bowen's disease and Paget's disease; liver cancer; lung cancer; lymphomas including Hodgkin's disease and lymphocytic lymphomas; neuroblastomas; oral cancer including squamous cell carcinoma; ovarian cancer including those arising from epithelial cells, stromal cells, germ cells and mesenchymal cells; pancreatic cancer; prostate cancer; rectal cancer; sarcomas including leiomyosarcoma, rhabdomyosarcoma, liposarcoma, fibrosarcoma, Ewing's sarcoma, and osteosarcoma; skin cancer including melanoma, Merkel cell carcinoma, Kaposi's sarcoma, basal cell carcinoma, and squamous cell cancer; testicular cancer including germinal tumors such as seminoma, non-seminoma (teratomas, choriocarcinomas), stromal tumors, and germ cell tumors; thyroid cancer including thyroid adenocarcinoma and medullar carcinoma; and renal cancer including adenocarcinoma and Wilms tumor. In some embodiments, cancer is a colon carcinoma, a pancreatic cancer, a breast cancer, an ovarian cancer, a prostate cancer, a squamous cell carcinoma, a cervical cancer, a lung carcinoma, a small cell lung carcinoma, a bladder carcinoma, a squamous cell carcinoma, a basal cell carcinoma, an adenocarcinoma, a sweat gland carcinoma, a sebaceous gland carcinoma, a papillary carcinoma, a papillary adenocarcinoma, a cystadenocarcinoma, a medullary carcinoma, a bronchogenic carcinoma, a renal cell carcinoma, a hepatocellular carcinoma, a bile duct carcinoma, a choriocarcinoma, a seminoma, a embryonal carcinoma, a Wilms' tumor, or a testicular tumor. In some embodiments, cancer is a lung carcinoma. In some embodiments, cancer is a breast carcinoma. In some embodiments, the cancer is believed to be of epithelial origin. In some embodiments, the cancer is of unknown cellular origin, but possesses at least one molecular or histological characteristic are associated with epithelial cells, such as the production of E-cadherin, cytokeratins or intercellular bridges.

In some embodiments agents capable of inhibiting endogenous EMT-inducing molecules and/or capable of inhibiting endogenous molecules that cooperate with EMT are administered to a subject who is also treated with one or more additional agents. In some embodiments an additional agent is a cancer chemotherapeutic agent. Non-limiting examples of cancer chemotherapeutics that can be useful with the methods disclosed herein for treating cancer include alkylating and alkylating-like agents such as Nitrogen mustards (e.g., Chlorambucil, Chlormethine, Cyclophosphamide, Ifosfamide, and Melphalan), Nitrosoureas (e.g., Carmustine, Fotemustine, Lomustine, and Streptozocin), Platinum agents (i.e., alkylating-like agents) (e.g., Carboplatin, Cisplatin, Oxaliplatin, BBR3464, and Satraplatin), Busulfan, Dacarbazine, Procarbazine, Temozolomide, ThioTEPA, Treosulfan, and Uramustine; Antimetabolites such as Folic acids (e.g., Aminopterin, Methotrexate, Pemetrexed, and Raltitrexed); Purines such as Cladribine, Clofarabine, Fludarabine, Mercaptopurine, Pentostatin, and Thioguanine; Pyrimidines such as Capecitabine, Cytarabine, Fluorouracil, Floxuridine, and Gemcitabine; Spindle poisons/mitotic inhibitors such as Taxanes (e.g., Docetaxel, Paclitaxel) and Vincas (e.g., Vinblastine, Vincristine, Vindesine, and Vinorelbine); Cytotoxic/antitumor antibiotics such anthracyclines (e.g., Daunorubicin, Doxorubicin, Epirubicin, Idarubicin, Mitoxantrone, Pixantrone, and Valrubicin), compounds naturally produced by various species of Streptomyces (e.g., Actinomycin, Bleomycin, Mitomycin, Plicamycin) and Hydroxyurea; Topoisomerase inhibitors such as Camptotheca (e.g., Camptothecin, Topotecan and Irinotecan) and Podophyllums (e.g., Etoposide, Teniposide); Monoclonal antibodies for cancer immunotherapy such as anti-receptor tyrosine kinases (e.g., Cetuximab, Panitumumab, Trastuzumab), anti-CD20 (e.g., Rituximab and Tositumomab), and others for example Alemtuzumab, Bevacizumab, and Gemtuzumab; Photosensitizers such as Aminolevulinic acid, Methyl aminolevulinate, Porfimer sodium, and Verteporfin; Tyrosine kinase inhibitors such as Cediranib, Dasatinib, Erlotinib, Gefitinib, Imatinib, Lapatinib, Nilotinib, Sorafenib, Sunitinib, and Vandetanib; serine/threonine kinase inhibitors, (e.g., inhibitors of Abl, c-Kit, insulin receptor family member(s), EGF receptor family member(s), mTOR, Raf kinase family, phosphatidyl inositol (PI) kinases such as PI3 kinase, PI kinase-like kinase family members, cyclin dependent kinase family members, Aurora kinase family), growth factor receptor antagonists, and others such as retinoids (e.g., Alitretinoin and Tretinoin), Altretamine, Amsacrine, Anagrelide, Arsenic trioxide, Asparaginase (e.g., Pegaspargase), Bexarotene, Bortezomib, Denileukin diftitox, Estramustine, Ixabepilone, Masoprocol, Mitotane, and Testolactone, Hsp90 inhibitors, proteasome inhibitors, HDAC inhibitors, angiogenesis inhibitors, e.g., anti-vascular endothelial growth factor agents such as Bevacizumab, matrix metalloproteinase inhibitors, pro-apoptotic agents (e.g., apoptosis inducers), anti-inflammatory agents, etc.

In some embodiments, methods of treatment described herein comprising inhibiting at least one endogenous EMT-inducing molecule and inhibiting at least one endogenous EMT-cooperating molecule are employed in the treatment of noncancerous diseases and conditions involving excessive or unwanted cell proliferation, such as keloids, scar formation, post-surgical adhesions, vascular stenosis, pathological neovascularization, fibrosis, etc.

In some embodiments methods of treatment can include a step of identifying or providing a subject suffering from or at risk of a disease or condition of interest. “At risk of” implies at increased risk of, relative to the risk such subject would have in the absence of one or more circumstances, conditions, or attributes of that subject, and/or relative to the risk that an average, healthy member of the population would have and/or relative to the risk that the subject had at a previous time. In some embodiments the subject is at least at a 20% increased risk (1.2 fold increased risk) of developing a disease or condition. Examples of conditions that may place a subject “at risk” will vary depending on the particular disease or condition and may include, but are not limited to, family history of the disease or condition; exposure or possible exposure (e.g., due to occupation, habits, etc.) to particular physical or chemical agents known or believed in the art to increase risk of developing the disease or condition; a mutation, genetic polymorphism, gene or protein expression profile, and/or presence of particular substances in the blood that is/are associated with increased risk of developing or having the disease relative to other members of the general population not having such mutation or genetic polymorphism; immunosuppression; presence of other diseases or conditions, age, surgery or other trauma; presence of symptoms; or any other condition that within the judgement and skill of the subject's health care provider place the subject at increased risk. In some embodiments a subject is suspected of having a disease or condition, e.g., as a result of having one or more risk factors and, typically, one or more symptoms or signs of the disease or condition. Any suitable methods may be employed to identify a subject in need of treatment a. For example, such methods may include clinical diagnosis based at least in part on symptoms, medical history (if available), physical examination, laboratory tests, imaging studies, immunodiagnostic assays, nucleic acid based diagnostics, etc. In some embodiments, diagnosis can at least in part be based on serology (e.g., detection of an antibody that specifically reacts with a marker associated with the disease).

In some embodiments the subject is at risk of cancer or cancer recurrence. A subject at risk of cancer may be, e.g., a subject who has not been diagnosed with cancer but has an increased risk of developing cancer as compared with an age-matched control, e.g., of the same sex. For example, the subject may have a risk at least 1.2 times that of a matched control. For example, a subject may be considered “at risk” of developing cancer if (i) the subject has a mutation, genetic polymorphism, gene or protein expression profile, and/or presence of particular substances in the blood, associated with increased risk of developing or having cancer relative to other members of the general population not having such mutation or genetic polymorphism; (ii) the subject has one or more risk factors such as having a family history of cancer, having been exposed to a mutagen, carcinogen or tumor-promoting agent or condition, e.g., asbestos, tobacco smoke, aflatoxin, radiation, chronic infection/inflammation, etc., advanced age. In some embodiments the subject has one or more symptoms of cancer but has not been diagnosed with the disease, e.g., the subject may be suspected of having cancer.

Agents and compositions disclosed herein and/or identified using a method described herein may be administered by any suitable means such as orally, intranasally, subcutaneously, intramuscularly, intravenously, intra-arterially, parenterally, intraperitoneally, intrathecally, intratracheally, ocularly, sublingually, vaginally, rectally, dermally, or by inhalation, e.g., as an aerosol. Depending upon the type of disease condition to be treated, agents may, for example, be inhaled, ingested, administered locally, or administered by systemic routes. Thus, a variety of administration modes, or routes, are available. The particular mode selected will typically depend on factors such as the particular agent selected, the particular condition being treated, and the dosage required for therapeutic efficacy. If multiple agents are administered they may be administered using the same or different routes in various embodiments. The methods, generally speaking, may be practiced using any mode of administration that is medically or veterinarily acceptable, meaning any mode that produces acceptable levels of efficacy without causing clinically unacceptable (e.g., medically or veterinarily unacceptable) adverse effects. In some embodiments, a route of administration is parenteral, which includes intravenous, intramuscular, intraperitoneal, subcutaneous, intraosseus, and intrasternal injection, or infusion techniques. In some embodiments, a route of administration is oral. In some embodiments, agents may be delivered to or near a site of diseased or damaged tissue or a tumor. In some embodiments, inhaled medications are of use. Such administration allows direct delivery to the lung, for example in subjects in need of treatment for lung cancer or lung fibrosis, although it could also be used to achieve systemic delivery of certain compounds. Several types of metered dose inhalers are regularly used for administration by inhalation. These types of devices include metered dose inhalers (MDI), breath-actuated MDI, dry powder inhaler (DPI), spacer/holding chambers in combination with MDI, and nebulizers. In some embodiments, intrathecal or intracranial administration may be of use, e.g., in a subject with a tumor of the central nervous system. Other appropriate routes and devices for administering therapeutic agents will be apparent to one of ordinary skill in the art.

Suitable preparations, e.g., substantially pure preparations, of one or more agents may be combined with one or more pharmaceutically acceptable carriers or excipients, etc., to produce an appropriate pharmaceutical composition suitable for administration to a subject. In some aspects, such pharmaceutically acceptable compositions are provided. The term “pharmaceutically acceptable carrier or excipient” refers to a carrier (which term encompasses carriers, media, diluents, solvents, vehicles, etc.) or excipient which does not significantly interfere with the biological activity or effectiveness of the active ingredient(s) of a composition and which is not excessively toxic to the host at the concentrations at which it is used or administered. Other pharmaceutically acceptable ingredients can be present in the composition as well. Suitable substances and their use for the formulation of pharmaceutically active compounds is well-known in the art (see, for example, “Remington's Pharmaceutical Sciences”, E. W. Martin, 19th Ed., 1995, Mack Publishing Co.: Easton, Pa., and more recent editions or versions thereof, such as Remington: The Science and Practice of Pharmacy. 21st Edition. Philadelphia, Pa. Lippincott Williams & Wilkins, 2005, for additional discussion of pharmaceutically acceptable substances and methods of preparing pharmaceutical compositions of various types. which are incorporated herein by reference in their entirety). Furthermore, agents and compositions may be used in combination with any agent or composition useful for treatment of a particular disease or condition of interest.

A pharmaceutical composition is typically formulated to be compatible with its intended route of administration. For example, preparations for parenteral administration include sterile aqueous or non-aqueous solutions, suspensions, and emulsions. Aqueous carriers include water, alcoholic/aqueous solutions, emulsions or suspensions, including saline and buffered media, e.g., sodium chloride solution, Ringer's dextrose, dextrose and sodium chloride, lactated Ringer's. Examples of non-aqueous solvents are propylene glycol, polyethylene glycol, vegetable oils such as olive oil, and injectable organic esters such as ethyl oleate. fixed oils, polyethylene glycols, glycerine, propylene glycol or other synthetic solvents; preservatives, e.g., antibacterial agents such as benzyl alcohol or methyl parabens; antioxidants such as ascorbic acid or sodium bisulfite; chelating agents such as ethylenediaminetetraacetic acid; buffers such as acetates, citrates or phosphates, and agents for the adjustment of tonicity such as sodium chloride or dextrose. pH can be adjusted with acids or bases, such as hydrochloric acid or sodium hydroxide. Such parenteral preparations can be enclosed in ampoules, disposable syringes or multiple dose vials made of glass or plastic.

Pharmaceutical compositions and compounds for use in such compositions may be manufactured under conditions that meet standards, criteria, or guidelines prescribed by a regulatory agency. For example, such compositions and compounds may be manufactured according to Good Manufacturing Practices (GMP) and/or subjected to quality control procedures appropriate for pharmaceutical agents to be administered to humans. Cells to be administered to a subject and compositions containing them may be maintained and handled as appropriate for such purpose in accordance with applicable standards, criteria, or guidelines.

For oral administration, compounds can be formulated readily by combining the active compounds with pharmaceutically acceptable carriers well known in the art. Such carriers enable compounds to be formulated as tablets, pills, dragees, capsules, liquids, gels, syrups, slurries, suspensions and the like, for oral ingestion by a subject to be treated. Suitable excipients for oral dosage forms are, e.g., fillers such as sugars, including lactose, sucrose, mannitol, or sorbitol; cellulose preparations such as, for example, maize starch, wheat starch, rice starch, potato starch, gelatin, gum tragacanth, methyl cellulose, hydroxypropylmethyl cellulose, sodium carboxymethylcellulose, and/or polyvinylpyrrolidone (PVP). If desired, disintegrating agents may be added, such as the cross linked polyvinyl pyrrolidone, agar, or alginic acid or a salt thereof such as sodium alginate. Optionally the oral formulations may also be formulated in saline or buffers for neutralizing internal acid conditions or may be administered without any carriers. Dragee cores are provided with suitable coatings. For this purpose, concentrated sugar solutions may be used, which may optionally contain gum arabic, talc, polyvinyl pyrrolidone, carbopol gel, polyethylene glycol, and/or titanium dioxide, lacquer solutions, and suitable organic solvents or solvent mixtures. Dyestuffs or pigments may be added to the tablets or dragee coatings for identification or to characterize different combinations of active compound doses.

Pharmaceutical preparations which can be used orally include push fit capsules made of gelatin, as well as soft, sealed capsules made of gelatin and a plasticizer, such as glycerol or sorbitol. The push-fit capsules can contain the active ingredients in admixture with filler such as lactose, binders such as starches, and/or lubricants such as talc or magnesium stearate and, optionally, stabilizers. In soft capsules, the active compounds may be dissolved or suspended in suitable liquids, such as fatty oils, liquid paraffin, or liquid polyethylene glycols. In addition, stabilizers may be added. Microspheres formulated for oral administration may also be used. Such microspheres have been well defined in the art.

Formulations for oral delivery may incorporate agents to improve stability in the gastrointestinal tract and/or to enhance absorption.

For administration by inhalation, a composition may be delivered in the form of an aerosol spray from a pressured container or dispenser which contains a suitable propellant, e.g., a gas such as carbon dioxide, a fluorocarbon, or a nebulizer. Liquid or dry aerosol (e.g., dry powders, large porous particles, etc.) can be used. Delivery of compositions using a nasal spray or other forms of nasal administration is contemplated.

For topical applications, pharmaceutical compositions may be formulated in a suitable ointment, lotion, gel, or cream containing the active components suspended or dissolved in one or more pharmaceutically acceptable carriers suitable for use in such composition.

For local delivery to the eye, the pharmaceutically acceptable compositions may be formulated as solutions or micronized suspensions in isotonic, pH adjusted sterile saline, e.g., for use in eye drops, or in an ointment.

Pharmaceutical compositions may be formulated for transmucosal or transdermal delivery. For transmucosal or transdermal administration, penetrants appropriate to the barrier to be permeated may be used in the formulation. Such penetrants are generally known in the art. Inventive pharmaceutical compositions may be formulated as suppositories (e.g., with conventional suppository bases such as cocoa butter and other glycerides) or as retention enemas for rectal delivery.

Direct administration to a tissue, e.g., a site of disease (e.g., at or near a tumor site) could be accomplished, e.g., by injection or by implanting a sustained release implant within the tissue. In some embodiments at least one of the agents is administered by release from an implanted sustained release device, by osmotic pump or other drug delivery device. A sustained release implant could be implanted at any suitable site. In some embodiments, a sustained release implant is used for prophylactic treatment of subjects at risk of developing a recurrent cancer or having a chronic condition (e.g., one that typically lasts for at least 6 months and often for years or indefinitely). In some embodiments, a sustained release implant or drug delivery device delivers therapeutic levels of the active agent(s) for at least 30 days, e.g., at least 60 days, e.g., up to 3 months, 6 months, or more. Compounds may be encapsulated or incorporated into particles, e.g., microparticles, microcapsules, or nanoparticles. Biodegradable, biocompatible polymers can be used, such as ethylene vinyl acetate, polyanhydrides, polyglycolic acid, PLGA, collagen, polyorthoesters, polyethers, and polylactic acid. Methods for preparation of such formulations will be apparent to those skilled in the art. For example, and without limitation, a number of particle-based delivery systems are known in the art for delivery of siRNA. The use of such compositions is contemplated. Liposomes or other lipid-based particles can also be used as pharmaceutically acceptable carriers.

It will be appreciated that pharmaceutically acceptable salts, esters, salts of such esters, prodrug, active metabolite, or any derivative which upon administration to a subject in need thereof is capable of providing a compound, directly or indirectly may be used in certain embodiments. The term “pharmaceutically acceptable salt” refers to those salts which are, within the scope of sound medical judgment, suitable for use in contact with the tissues of humans and/or lower animals without undue toxicity, irritation, allergic response and the like, and which are commensurate with a reasonable benefit/risk ratio. A wide variety of appropriate pharmaceutically acceptable salts are well known in the art. Pharmaceutically acceptable salts include, but are not limited to, those derived from suitable inorganic and organic acids and bases.

A variety of approaches can be used to increase plasma half-life, reduce clearance, or otherwise modify properties of an agent, e.g., a nucleic acid, polypeptide, or small molecule, if desired. See, e.g., Werle M, et al., Strategies to improve plasma half life time of peptide and protein drugs. Amino Acids 30(4):351-67, 2006 and Jevsevar S, et al, PEGylation of Therapeutic Proteins, Biotechnology Journal, 5(1): 113-128, 2010 for reviews discussing some of these approaches. For example, polymers such as polyalkylene glycol, e.g., polyethylene glycol, may be conjugated to an agent to increase circulation time, increase stability, half-life, or desirably modify other properties. Other approaches include conjugation to or fusion with an antibody Fc domain, albumin, or albumin-binding peptide. Methods of preparing conjugates and reagents of use in such methods are known to those of ordinary skill in the art. Exemplary methods and reagents are described in Hermanson, G., Bioconjugate Techniques, 2^{nd} ed., Academic Press, 2008. As will be appreciated, modified agents can be used in any of a variety of ex vivo or in vivo methods or in compositions or kits described herein.

Pharmaceutical compositions, when administered to a subject, are preferably administered for a time and in an amount sufficient to treat the disease or condition for which they are administered. Therapeutic efficacy and toxicity of active agents can be assessed by standard pharmaceutical procedures in cell cultures or experimental animals. The data obtained from cell culture assays and animal studies can be used in formulating a range of dosages suitable for use in humans or other subjects. Different doses for human administration can be further tested in clinical trials in humans as known in the art. The dose used may be the maximum tolerated dose or a lower dose. A therapeutically effective dose of an active agent in a pharmaceutical composition may be within a range of about 0.001 to about 100 mg/kg body weight, about 0.01 to about 25 mg/kg body weight, about 0.1 to about 20 mg/kg body weight, about 1 to about 10 mg/kg. Other exemplary doses include, for example, about 1 μg/kg to about 500 mg/kg, about 100 μg/kg to about 5 mg/kg). In some embodiments a single dose is administered while in some embodiments multiple doses are administered. Those of ordinary skill in the art will appreciate that appropriate doses in any particular circumstance depend upon the potency of the agent(s) utilized, and may optionally be tailored to the particular recipient. The specific dose level for a subject may depend upon a variety of factors including the activity of the specific agent(s) employed, the particular disease or condition and its severity, the age, body weight, general health of the subject, etc. Similarly, the number of cells to be administered in a cell-based therapy can be determined by those of ordinary skill in the art based on such considerations, the type of cell administered, etc. In various embodiments the number of cells administered is thousands, tens to hundreds of thousands, millions, or more.

It may be desirable to formulate pharmaceutical compositions, particularly those for oral or parenteral compositions, in unit dosage form for ease of administration and uniformity of dosage. Unit dosage form, as that term is used herein, refers to physically discrete units suited as unitary dosages for the subject to be treated; each unit containing a predetermined quantity of active agent(s) calculated to produce the desired therapeutic effect in association with an appropriate pharmaceutically acceptable carrier. It will be understood that a therapeutic regimen may include administration of multiple unit dosage forms over a period of time, which can extend over days, weeks, months, or years. In some embodiments, treatment may be continued indefinitely, e.g., in order to achieve prophylaxis or in the case of a chronic disease. A subject may receive one or more doses a day, or may receive doses every other day or less frequently, within a treatment period.

### EXAMPLES

### Example 1

### Expression of EMT-Inducing Transcription Factors in Mammary Stem Cells

The previously demonstrated connection between the EMT and certain MaSC properties showed that TFs that are able to induce passage though an EMT program (EMT-TFs) could also serve as key regulators for conferring SC traits on differentiated mammary epithelial cells (MECs). We wished to extend this work by developing direct functional proof of the connection between passage through an EMT and the acquisition of SC traits in a normal epithelial tissue in vivo. To do so, we utilized primary murine mammary epithelial cells, as the murine mammary gland reconstitution assay offers a robust measure of SC activity. The mammary gland represents a useful model system for studying the regulation of epithelial SCs, as it contains a small subpopulation of cells with robust SC activity. Implantation of a single murine mammary stem cell (MaSC) into the murine mammary fat pad, which represents the stromal component of the normal mammary gland, is sufficient to generate an entire mammary ductal tree. This in vivo regeneration assay makes the murine mammary gland a powerful system for dissecting the regulatory mechanisms that control epithelial SCs and offers a stringent test of multi-lineage stemness.

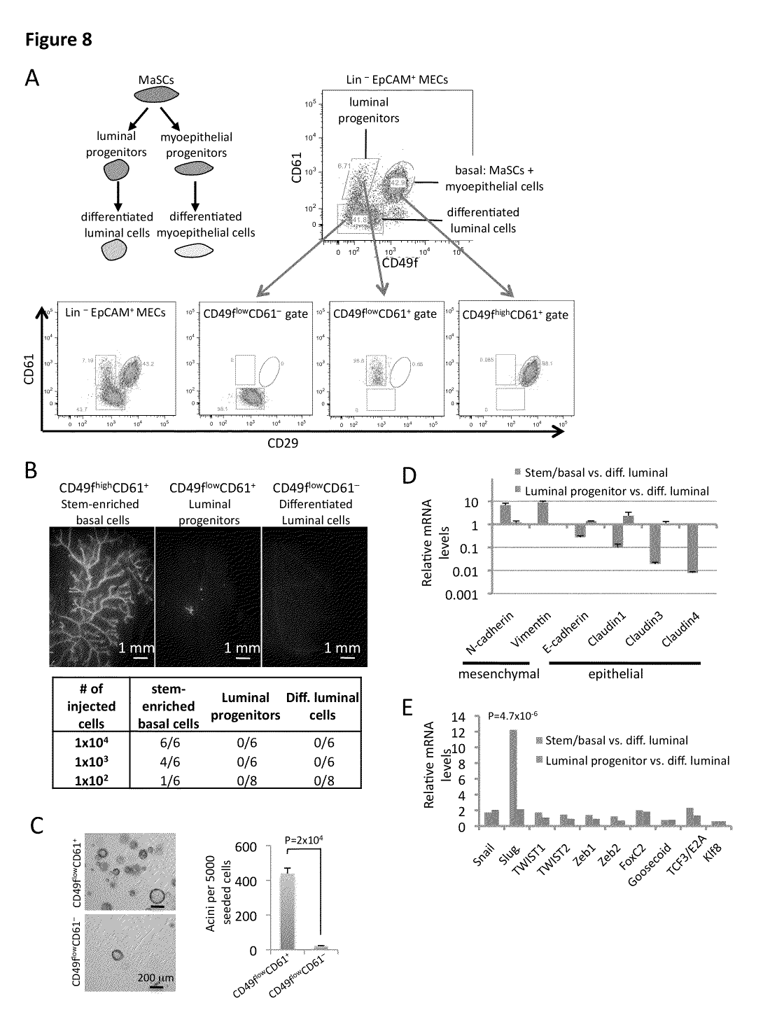

To begin, we resolved distinct subpopulations of freshly isolated murine MECs using cell-surface antigenic markers CD49f and CD61, which separated murine MECs into three populations as effectively as the published CD29 and CD61 method (Asselin-Labat et al., 2007). These populations were CD49f^{high}CD61⁺ MaSC-enriched basal cells, CD49f^{low}CD61⁺ luminal progenitors, and CD49f^{low}CD61⁻ differentiated luminal cells (FIG. 8A). We confirmed that we could efficiently separate MaSC-enriched, luminal progenitor and differentiated luminal cell populations by using this approach (FIGS. 8B and C).

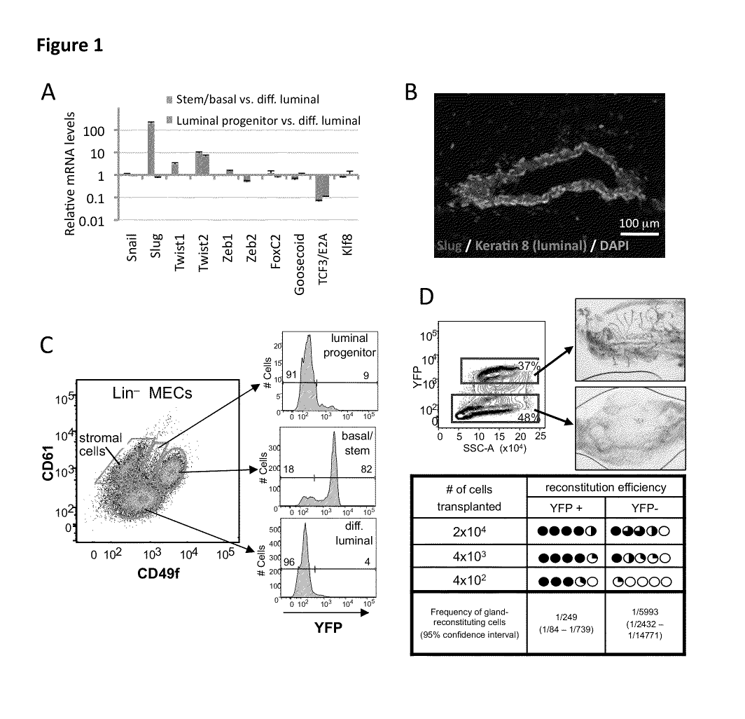

We proceeded to measure the expression in these MEC populations of various mRNAs encoding ten previously described EMT-TFs. Among this group of EMT-TFs, only Slug was highly over-expressed (˜200-fold) in the MaSC-enriched basal population relative to other populations (FIG. 1A). Analysis of published microarray data from various human MEC subpopulations confirmed that Slug was also the most highly expressed EMT-TF in the human MaSC-enriched basal cell population (FIG. 8E) (Lim et al., 2010). The Slug protein was also specifically expressed in the nuclei of basal cells in the mammary epithelium, as determined by immunofluorescence (FIG. 1B). This cell layer has been reported to contain virtually all of the MaSCs (Shackleton et al., 2006; Stingl et al., 2006a). However, we noted that a relatively high proportion of basal cells expressed Slug, suggesting that other basal cells, in addition to MaSCs, also express the Slug EMT-TF.

To demonstrate unequivocally that the MaSCs residing among the larger basal cell population do indeed express Slug, we generated a transgenic mouse line (Slug-YFP mice) in which expression of a yellow fluorescent protein (YFP) gene was driven by the endogenous Slug promoter. Consistent with the immunofluorescence data, only CD49f^{high}CD61⁺ basal cells expressed Slug-YFP, while luminal progenitors and differentiated luminal cells did not express this EMT-TF to a significant extent (FIG. 1C). To examine whether MaSCs were enriched in Slug-YFP-positive MECs, we transplanted FACS-sorted YFP-positive and -negative MECs into cleared mammary fat pads at limiting dilutions. We calculated the frequency of mammary gland-reconstituting cells to be 1/250 in the Slug-YFP-positive cells versus 1/6000 in the Slug-YFP-negative cells (FIG. 1D). These results demonstrated that Slug is strongly expressed in MaSCs. However, these correlative observations did not indicate whether Slug plays a functional role either in inducing the formation of MaSC or maintaining their residence in the SC state.

### Example 2

### Establishing an Improved In Vitro MaSC Assay

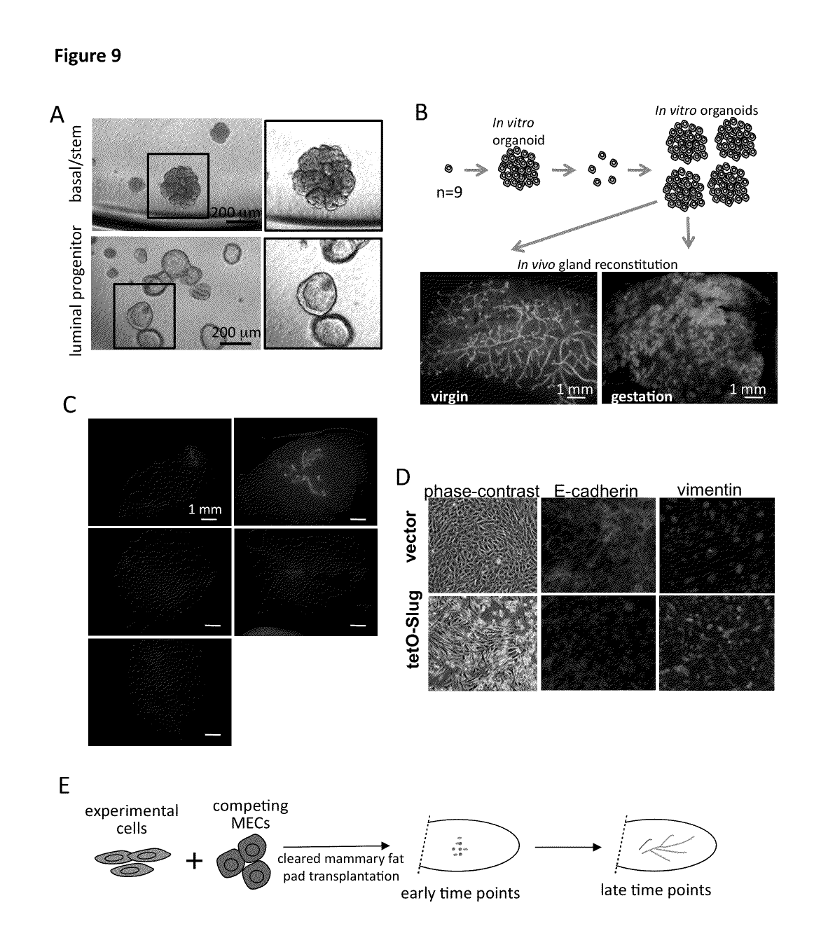

To facilitate MaSC studies, we sought to establish a more robust in vitro MaSC assay by modifying existing Matrigel culture methods (Lim et al., 2009; Shackleton et al., 2006; Stingl et al., 2006a). We used a ROCK inhibitor to increase organoid-forming efficiency. We also reduced Matrigel concentration to 5%, which did not affect organoid formation, but facilitated passage of organoids. In these 3-dimensional cultures, ˜3% of MaSC-enriched basal cells formed solid organoids, whereas luminal progenitors formed acini with hollow lumina at high efficiencies (˜13%), but rarely formed solid organoids (<0.1%) (FIG. 9A). This is consistent with previous observations that MaSCs have an ability to form solid organoids, whereas luminal progenitors form acinar structures in Matrigel cultures (Lim et al., 2009; Shackleton et al., 2006; Stingl et al., 2006a).

To demonstrate directly that the solid organoids indeed contained MaSCs, we examined whether individual organoids isolated from these cultures could reconstitute mammary ductal trees in vivo, a stringent test that has not been performed in previous studies using Matrigel culture assays. We found about 70% of organoid cultures generated from single cells yielded full mammary ductal tree reconstitution (FIG. 9B). In addition, when recipient mice were impregnated, these reconstituted mammary ductal trees underwent robust alveologenesis, a differentiation process that gives rise to milk-secreting alveoli (FIG. 9B). In contrast to solid organoids, acini formed by luminal progenitor cells in the improved organoid culture could not reconstitute mammary ductal trees upon transplantation into cleared mammary fat pads (FIG. 9C). These data showed that almost all solid organoids formed in the improved Matrigel culture contained functional MaSCs and that our Matrigel organoid assay could serve as a specific in vitro assay for the presence of MaSCs.

### Example 3

### Effects of Ectopic Expression of Slug on MaSC Activity In Vivo

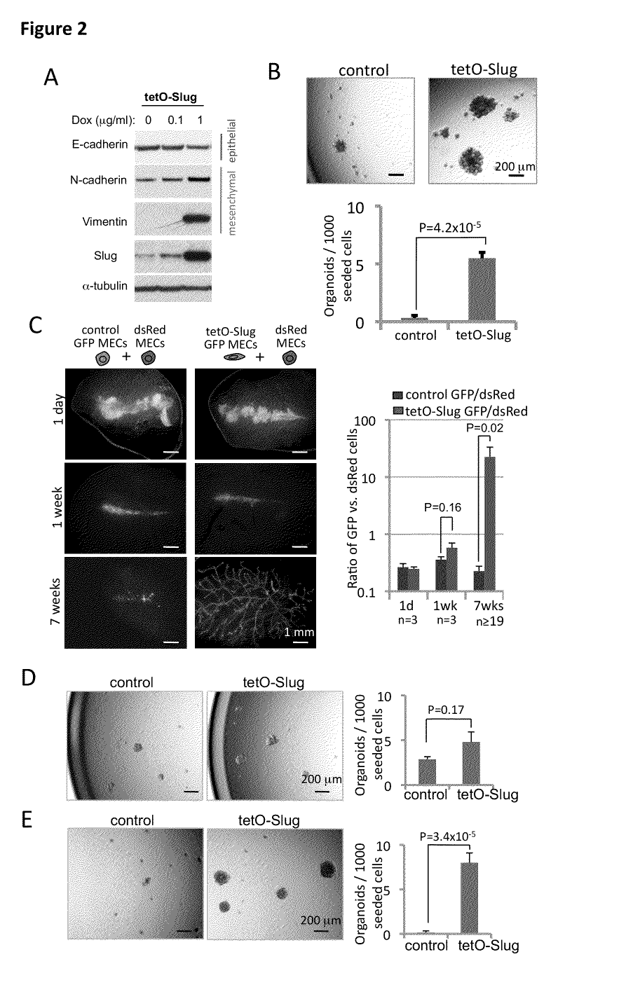

To examine a functional role for Slug in MaSC induction, we transiently expressed Slug in primary MECs by using a tetracycline-inducible lentiviral expression vector. Use of this vector to induce Slug in primary MECs resulted in a robust EMT, as judged by decreased expression of epithelial markers and increased expression of mesenchymal markers (FIG. 2A and FIG. 9D). To examine the effect of Slug expression on MaSC induction, we expressed Slug transiently in primary MECs in monolayer culture for 7 days and then transferred these cells to organoid culture in the absence of doxycycline and thus in the absence of further ectopic Slug expression. We observed that MECs that had previously expressed Slug transiently generated 17 times more organoids than did control-vector-expressing MECs (FIG. 2B).

We further measured Slug-induced formation of MaSCs, now using the more stringent in vivo cleared mammary fat pad reconstitution assay (DeOme et al., 1959). In a competitive reconstitution analysis, we expressed Slug or the control tetracycline-inducible vector in GFP-expressing primary MECs for 5 days in monolayer culture, and then implanted these cells (1×10⁵) with an equal number of admixed dsRed-expressing MECs that had not been virally transduced into cleared mammary fat pads. The animals were treated with doxycycline for 7 additional days after the transplantation and then maintained on a doxycycline-free diet. We analyzed the implanted fat pads at different time points post-implantation in order to monitor the short-term and long-term effects of transient Slug expression on initial engraftment and subsequent ductal morphogenesis (see FIG. 9E for a schematic description of this experiment).

At days 1 and 7 following implantation, the Slug and control-vector GFP-expressing cells contributed to engrafted murine MECs to comparable extents, indicating that a history of exposure to Slug had not affected the ability of MECs to survive the initial rigors of implantation. However, at 7 weeks post implantation, MECs that had experienced transient Slug exposure at the beginning of the experiment generated elaborate mammary ductal trees, while the control-vector-expressing cells formed only rudimentary structures (FIG. 2C). The Slug-exposed MECs generated mammary ductal trees 35-fold more efficiently than did the control-vector-expressing cells, as measured by the ratio of GFP-versus dsRed-expressing MECs engrafted in the fat pads (FIG. 2C). Together, these results demonstrated that transient expression of Slug dramatically increased the representation of gland-repopulating MaSCs.

### Example 4

### Slug Induces MaSCs from Luminal Progenitors but not Differentiated Luminal Cells

In the experiments described above, we reasoned that Slug exposure could function either by expanding a pre-existing population of MaSCs or by converting non-SCs into SCs. To distinguish between these two alternative mechanisms, we fractionated primary MECs into MaSC-enriched basal cells, luminal progenitors and differentiated luminal cells, as described in FIG. S1A, and subsequently examined the effect of ectopic Slug expression on inducing MaSC activity in each of these three purified cell populations.

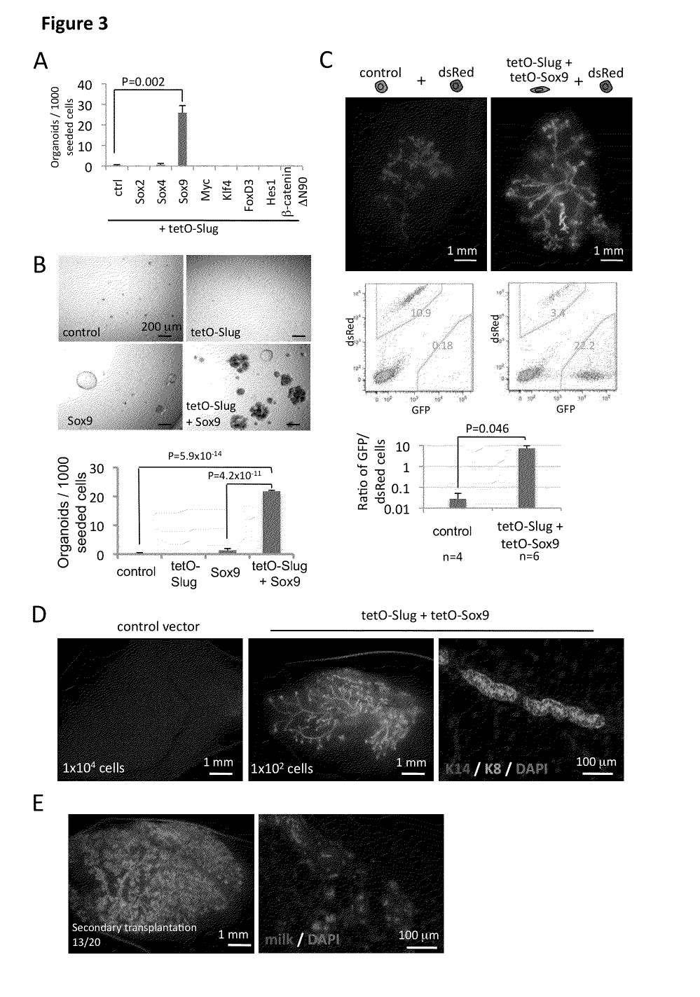

Transient expression of Slug in the MaSC-enriched basal cells for 7 days increased organoid-forming ability by less than 2-fold, suggesting that ectopic Slug expression only modestly increased the pool of MaSCs in a population of cells that already contained significant numbers of endogenous MaSCs (FIG. 2D). In contrast, similar transient expression of Slug in luminal progenitors led to a 50-fold increase in the representation of organoid-forming cells compared to vector-control cells treated in parallel, indicating that Slug could convert luminal progenitors into MaSCs (FIG. 2E). However, transient Slug expression in the differentiated luminal cells failed to induce any organoid-forming cells whatsoever (FIG. 3B). This indicated that expression of Slug on its own, while capable of inducing luminal progenitors to enter into the MaSC state, failed to induce differentiated luminal cells to do so.

### Example 5

### Cooperation of Sox9 with Slug in the Formation of Mammary Stem Cells

We reasoned that the inability of Slug to induce the formation of MaSCs by differentiated luminal cells might be due to the fact that Slug required the cooperation of one or more additional factors. To identify such cooperating factors, we selected eight TFs that had been implicated previously by others to play important roles in either embryonic or adult stem cell biology, or had been shown to cooperate with Slug in certain early developmental processes (FIG. 3A) (Cheung et al., 2005; Pece et al., 2010; Takahashi and Yamanaka, 2006). We co-expressed each of these factors together with Slug in pre-sorted differentiated luminal cells to determine whether any of them could collaborate with Slug to induce organoids in the Matrigel culture assay.

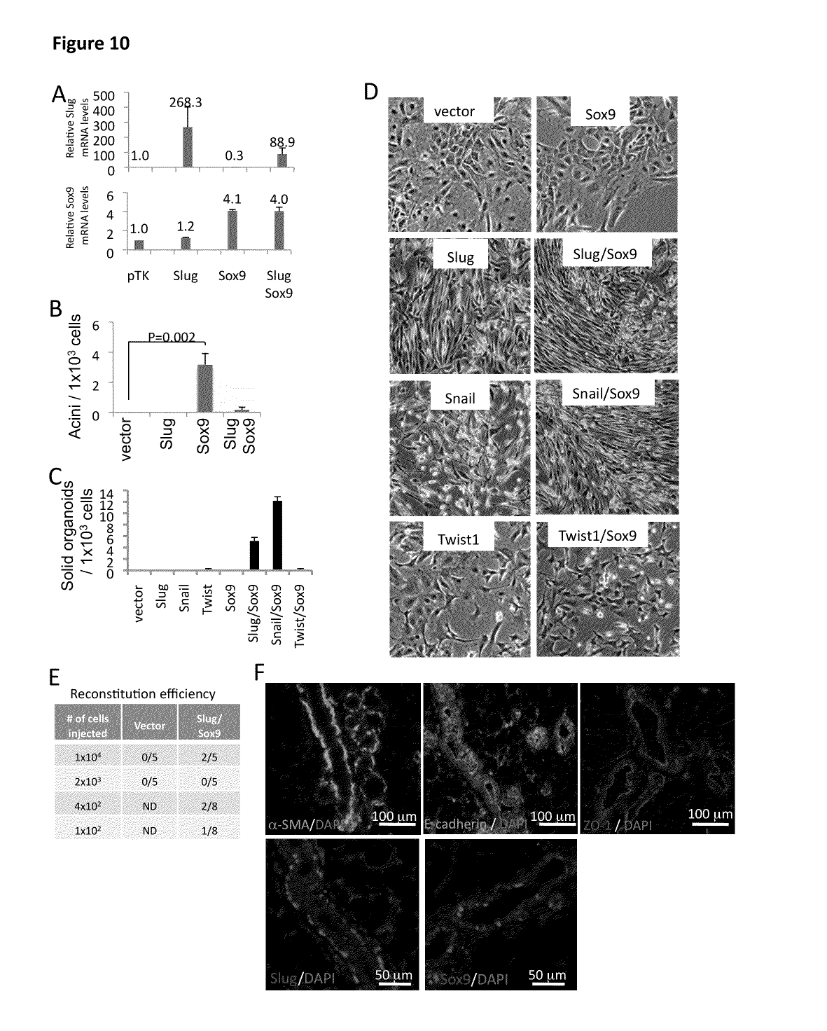

Among these eight TFs, SRY (sex determining region Y)-box 9 (Sox9) was particularly effective in inducing together with Slug the formation of solid organoids by differentiated luminal cells. In contrast, the other co-expressed factors failed to collaborate with Slug to induce organoids (FIG. 3A). Expression of Sox9 on its own in differentiated luminal cells had a far smaller effect in inducing organoid-forming cells relative to the effects of co-expressing Slug and Sox9 (FIG. 3B). We concluded that Slug and Sox9 could collaborate to induce differentiated luminal cells to enter the MaSC state. Interestingly, the expression in differentiated luminal cells of Sox9 by itself led to the formation of acini with hollow lumina (FIG. 3B and FIG. 10B), which are indicative of luminal progenitor activity (FIG. 9A) (Lim et al., 2009; Shackleton et al., 2006; Stingl et al., 2006a).

Our previous work had shown that the expression of other EMT-TFs, such as Snail and Twist1, could induce stem-like, cells in immortalized human MECs (Mani et al. 2008). We therefore tested whether these two EMT-TFs could also cooperate with Sox9 to induce MaSC formation in primary mouse MECs. Interestingly, while Snail could replace Slug to induce MaSCs in cooperation with Sox9, Twist1 failed to do so (FIG. 10C). In fact, Twist1 also could not induce a robust EMT in monolayer cultures of primary mouse MECs, whereas Slug and Snail could indeed do so (FIG. 10D). This inefficient EMT induction might well be the reason why Twist1 failed to induce MaSCs. These results suggested that potent EMT-TFs other than Slug, such as the related Snail TF, could potentially also cooperate with Sox9 to induce SC formation.

We further tested whether Slug and Sox9 could collaborate to convert differentiated luminal cells into MaSCs capable of reconstituting cleared mammary fat pads. To do so, we introduced tetracycline-inducible Slug and Sox9 expression vectors into sorted GFP-expressing differentiated luminal cells and induced the expression of both genes for 5 days in monolayer culture. The resulting cell populations were mixed with an equal number (1×10⁵) of unsorted dsRed-expressing MECs and subjected to the in vivo competitive reconstitution assay without further doxycycline treatment. As anticipated, the control-vector-transduced differentiated luminal cells exhibited no reconstituting ability (FIG. 3C). In contrast, the Slug/Sox9-exposed cells acquired robust gland-reconstituting activity, which was 7-fold higher than that of co-mixed unsorted dsRed-expressing primary MECs, which did contain an endogenous MaSCs subpopulation (FIG. 3C).

To directly measure the frequency of MaSCs, we transplanted the Slug/Sox9-exposed differentiated luminal cells into cleared mammary fat pads in limiting dilutions without admixing these cells with competing dsRed-labeled MECs. While control-vector-transduced cells failed to reconstitute, even when injected as an inoculum of 1×10⁴ cells, the Slug/Sox9-exposed cells generated fully developed mammary ductal trees when as few as 100 of these cells were implanted (FIG. 3D and FIG. 10E). Immunofluorescence analyses showed that these ductal outgrowths were bilayer structures composed of cytokeratin 14- and α-smooth muscle actin-positive myoepithelial cells and cytokeratin 8-positive luminal cells (FIGS. 3D and 10F). These results showed once again that transient expression of Slug and Sox9 in differentiated luminal cells sufficed to convert them into bipotential MaSCs.

To examine whether MaSCs generated from differentiated luminal cells exhibited long-term reconstituting ability, we prepared small fragments (˜1 mm) from primary outgrowths engrafted by Slug/Sox9-exposed differentiated luminal cells and re-implanted these fragments once again into cleared mammary fat pads. We found that 13 out of 20 such transplantations generated full secondary gland reconstitution (FIG. 3E). Furthermore, when the recipient mice were impregnated, these reconstituted mammary ductal trees generated large numbers of milk-secreting alveoli, indicating that the reconstituted mammary glands retained full differentiation potential (FIG. 3E). These data indicated that transient Slug and Sox9 expression was sufficient to induce long-term repopulating MaSCs, and that such MaSCs were able to self-renew without the need of continuous expression of exogenous Slug and Sox9.

### Example 6

### Effect of Sox9 on Mammary Stem Cell Induction in Basal Cells

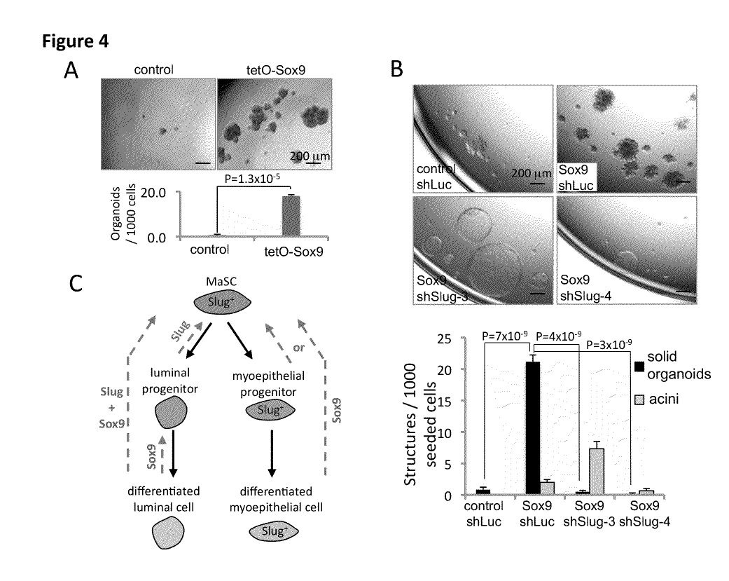

The above results suggested that Slug and Sox9, acting in concert, could function as master regulators of the MaSC state. If this were indeed the case, then these two TFs should be able to induce the formation of MaSCs in MECs prepared from various mammary epithelial lineages. In fact, as mentioned earlier, we found that the CD49f^{high}CD61⁺ basal cells, which contain both MaSCs and myoepithelial cells, already expressed significant levels of endogenous Slug and had mesenchymal attributes (FIG. 1 and FIG. 8D). This suggested that ectopic Sox9 expression on its own in these basal cells might suffice to induce MaSCs by acting together with the endogenously expressed Slug. Indeed, we detected a 26-fold increase in the number of organoid-forming cells when Sox9 was transiently overexpressed on its own in the basal cells for 5 days prior to organoid culture (FIG. 4A).

To examine the requirement for endogenous Slug in the Sox9-mediated MaSC induction, we knocked down Slug in these basal cells concomitant with constitutive Sox9 overexpression. In this case, the induction of organoid-forming cells was completely suppressed; in contrast, basal cells expressing Sox9 and a control shRNA readily formed organoids (FIG. 4B). This revealed that ectopically expressed Sox9 could collaborate with endogenously expressed Slug to induce organoid formation in basal cells, and that both of these factors were required for the outgrowth of these structures. As the basal cell population contained pre-existing MaSCs, which cannot to be separated from myoepithelial cells with currently available markers, we could not distinguish whether in this case Sox9 converted myoepithelial cells into MaSCs or expanded a pre-existing MaSC population. Similarly, we could not distinguish whether Sox9 converted myoepithelial progenitors or differentiated myoepithelial cells into MaSCs.

Of note, when the endogenously expressed Slug was knocked down in the basal cells, Sox9 overexpression induced the formation of acinar structures instead of solid organoid structures, which were otherwise induced by Sox9 in unperturbed basal cells (FIG. 4B). This indicated that the inhibitory effect of Slug knockdown on MaSC induction was not simply caused by non-specific cytostatic or cytotoxic effects, because the knockdown of Slug still permitted the robust outgrowth of acinar structures. This also suggested that, in the absence of endogenous Slug, ectopic Sox9 expression might cause trans-differentiation of basal cells into luminal progenitor cells, since formation of acinar structures is indicative of the activity of luminal progenitor cells (Lim et al., 2009; Stingl et al., 2006a)^{⊥}. Taken together with the above findings that Sox9 overexpression alone in differentiated luminal cells induced acinus-forming cells (FIG. 10B), these results suggested a role of Sox9 in luminal progenitor cells, in addition to its function in inducing the formation of MaSCs. This evidence allowed us, in turn, to propose the hierarchical scheme illustrated in FIG. 4C, in which co-expression of Slug and Sox9 in either luminal or basal MECs sufficed to convert them to MaSCs.

### Example 7

### Role of Slug and Sox9 in the Maintenance of Endogenous Mammary Stem Cells

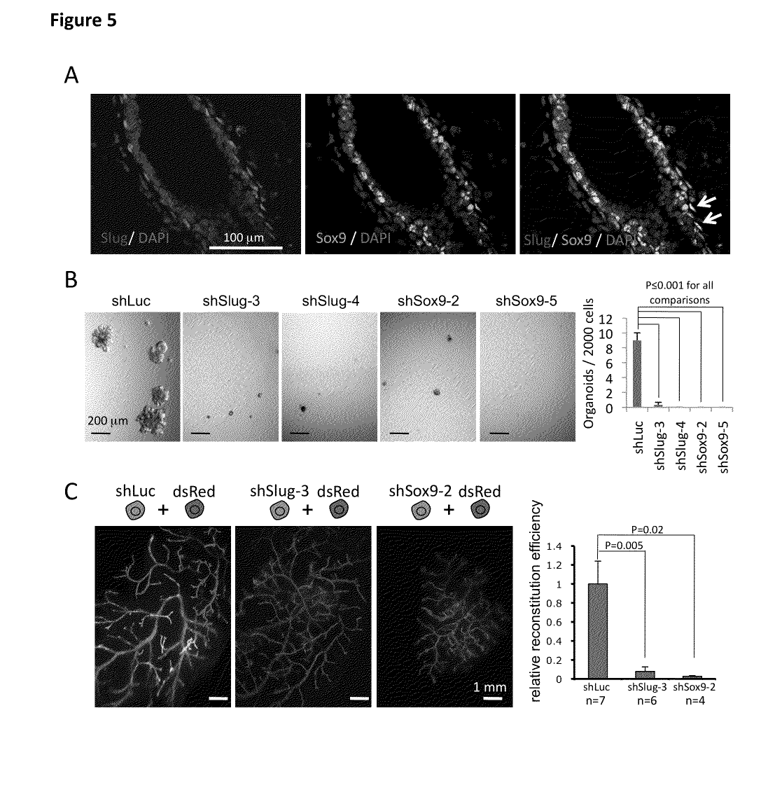

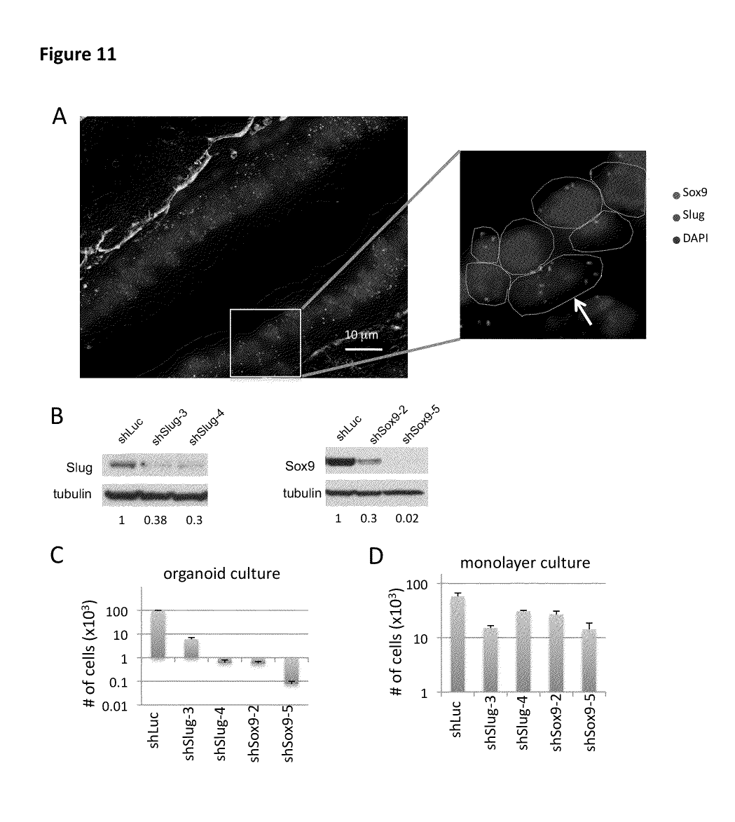

The combined effects of Slug and Sox9 on the induction of MaSC formation suggested that continued coexpression of these two TFs might also be required to maintain naturally arising MaSCs in this system. Consistent with this notion, we detected a subset of basal cells in the murine mammary gland expressing high levels of both Slug and Sox9 by using immunofluorescence (FIG. 5A) or single-molecule fluorescence in situ hybridization (FIG. 11A), suggesting Slug and Sox9 were co-expressed by naturally arising MaSCs in vivo.

To further examine the function of Slug and Sox9 in MaSCs, we knocked down the expression of either Slug or Sox9 in unsorted primary MECs with shRNAs (FIG. 11B). The Slug shRNAs reduced the number of organoid-forming cells by more than 27-fold, while the Sox9 shRNAs reduced the number of organoid-forming MECs to a non-detectable level (FIG. 5B). Consistent with the inhibition of organoid formation, overall cell number in 3D organoid culture was reduced by almost 100-fold by either the Slug or the Sox9 knockdown (FIG. 11C). In contrast, knockdown of either of these genes reduced the number of primary MECs in monolayer cultures by only 2- to 4-fold during the same period (FIG. 11D), suggesting that the effect of Slug and Sox9 inhibition on MaSC activity in organoid culture was not primarily due to general inhibition of cell proliferation or survival. Furthermore, knockdown of Sox9 or Slug also blocked the in vivo gland-reconstituting activity of primary, unfractionated MECs, demonstrating that Slug and Sox9 are both required for maintaining the endogenous MaSC population (FIG. 5C).

### Example 8

### Distinct Gene Expression Programs Activated by Slug and Sox9

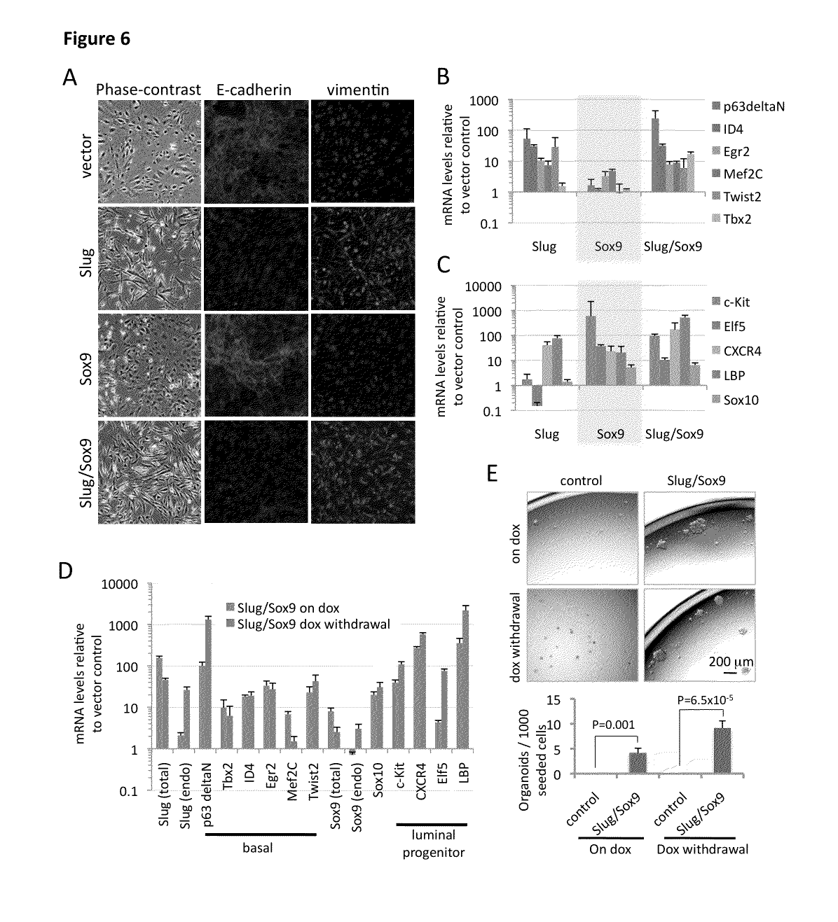

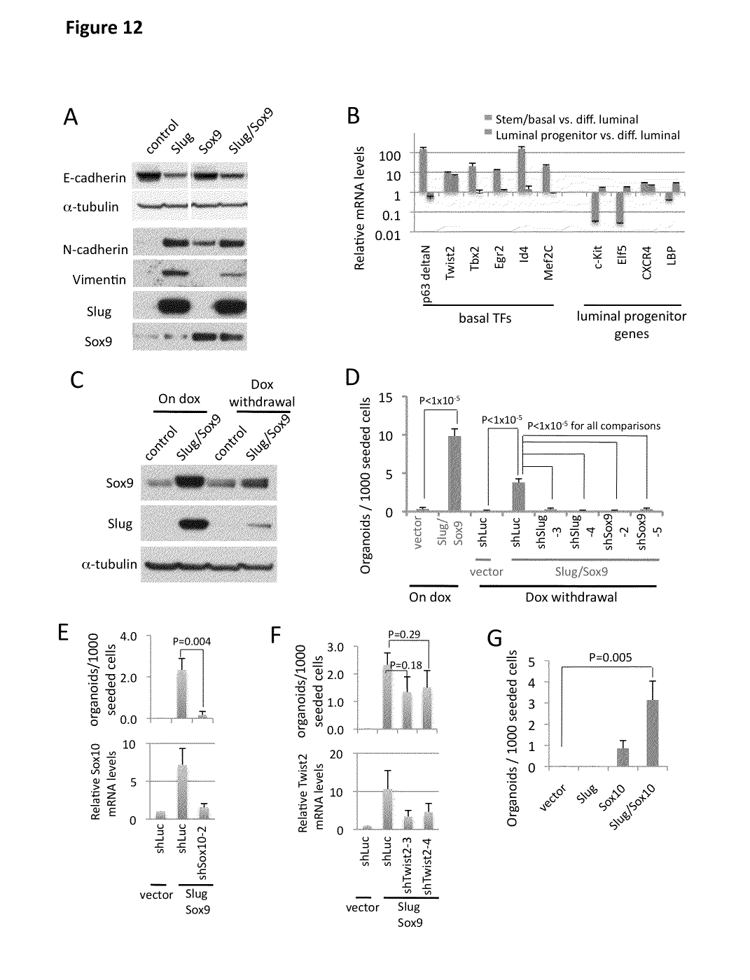

The above results demonstrated that Slug and Sox9 act cooperatively as master regulators of the formation and maintenance of MaSCs. To gain insight into how Slug and Sox9 succeed in doing so, we examined whether they promoted the EMT synergistically, in light of previous work that showed a connection between passage through an EMT and entrance into a SC-like state (Mani et al., 2008; Morel et al., 2008). Accordingly, we expressed Slug and Sox9 either individually or in combination in differentiated luminal cells for 5 days. As shown earlier, expression of Slug alone in these cells propagated in monolayer culture induced a robust EMT (FIG. 6A and FIG. 12A). In contrast, expressing Sox9 alone had only a modest effect on inducing the EMT, and when co-expressed with Slug, Sox9 did not potentiate EMT-inducing powers of Slug (FIG. 6A and FIG. 12A). This result suggested that while Slug may contribute to SC determination by inducing an EMT, Sox9 activates a complementary, ostensibly distinct cell-biological program that cooperates with the EMT program to enable entrance into the SC state.

Gene expression microarray analyses had previously identified signature genes of various mammary epithelial cell subpopulations in both human and mouse (Lim et al., 2010). We validated the expression of these signature genes in the corresponding murine MEC subpopulations by using qRT-PCR (FIG. 12B). Responding to these findings, we examined whether Slug and/or Sox9 regulate cellular states by altering the expression of these signature-associated genes. We found that expression of Slug in differentiated luminal cells upregulated expression of mRNAs encoding five of six basal cell-associated TFs by at least 7-fold (FIG. 6B), consistent with a previously suggested role of Slug in maintaining basal-like phenotypes (Proia et al., 2010). In contrast to the behavior of Slug, forced Sox9 expression in differentiated luminal cells specifically upregulated the expression of genes associated with luminal progenitors. Thus, expression of all four luminal progenitor genes was increased by more than 20-fold in Sox9-expressing cells (FIG. 6C). In addition, Sox9 induced Sox10 expression by more than 5-fold (FIG. 6C). When Slug and Sox9 were co-expressed in differentiated luminal cells, the gene expression signatures of both basal cells and luminal progenitors were concomitantly upregulated (FIGS. 6B and C). These results reinforced and extended earlier findings that Slug and Sox9 regulated basal and luminal lineage programs, respectively. These two programs may contribute portions of the biological functions required to enter into and reside stably within the MaSC state.

Because transient concomitant expression of Slug and Sox9 sufficed to induce entrance into the MaSC state, we asked whether the Slug- and Sox9-induced gene expression programs remained active in the resulting MaSCs even after the ectopically expressed Slug and Sox9 TFs had been turned off. Consequently, we transiently expressed Slug and Sox9 in differentiated luminal cells for 6 days in monolayer culture and then turned off the expression of Slug and Sox9 for a subsequent 6 days via doxycycline withdrawal. At this time point, we confirmed that expression of the exogenous TFs had been successfully silenced (FIG. 6D). However, endogenous basal and luminal progenitor signature genes remained actively expressed (FIG. 6D). Interestingly, expression of exogenous Slug and Sox9 led, in turn, to the induction of endogenously expressed EMT-TFs, including Twist2 and Slug itself, and endogenous Sox factors, including Sox9 and its close paralog Sox10 (FIG. 6D and FIG. 12C). Hence, the ectopically expressed Slug and Sox9 induced expression of their corresponding endogenous counterparts or paralogs, forming a self-reinforcing auto-regulatory network that contributed to maintenance of the SC program long after the exogenous factors had been silenced.

Consistent with the persistent activation of the SC gene expression program, the representation of SCs in Slug/Sox9-exposed cells in monolayer culture remained stable after exogenous Slug and Sox9 were turned off. Thus, six days after turning off the expression of exogenous Slug and Sox9, cells that had previously experienced these TFs exhibited a similar organoid-forming efficiency as they did when introduced into organoid culture immediately after halting expression of these exogenous TFs (FIG. 6E). We further tested whether continuous expression of endogenous Slug and Sox9 was required for maintaining the induced MaSCs. After differentiated luminal cells were exposed to exogenous Slug and Sox9 for 6 days, we transduced the cells with shRNA vectors directed against either Slug or Sox9 and cultured the cells in doxycycline-free media in monolayer for 6 days to inhibit expression of the endogenous Slug or Sox9 proteins that may have been induced. We found that knocking down either Slug or Sox9 reduced the numbers of induced MaSCs by more than 10-fold, as gauged by organoid culture (FIG. 12D).

We further examined the functional relevance of Sox9- and Slug-induced TFs, specifically Sox10 and Twist2, in MaSC induction, as measured by organoid culture. To do so, we knocked down either Sox10 or Twist2 while concomitantly expressed Slug and Sox9 in differentiated luminal cells. The inhibition of Sox10 led to more than 90% reduction in organoid formation (FIG. 12E), while the inhibition of Twist2 suppressed organoid formation modestly (FIG. 12F). Conversely, expressing Sox10 together with Slug could induce formation of organoids from differentiated luminal cells (FIG. 12G), suggesting that Sox10 acted as a downstream effector of Sox9 in the MaSC induction.

### Example 9

### Roles of Slug and Sox9 in Breast Cancer Stem Cells

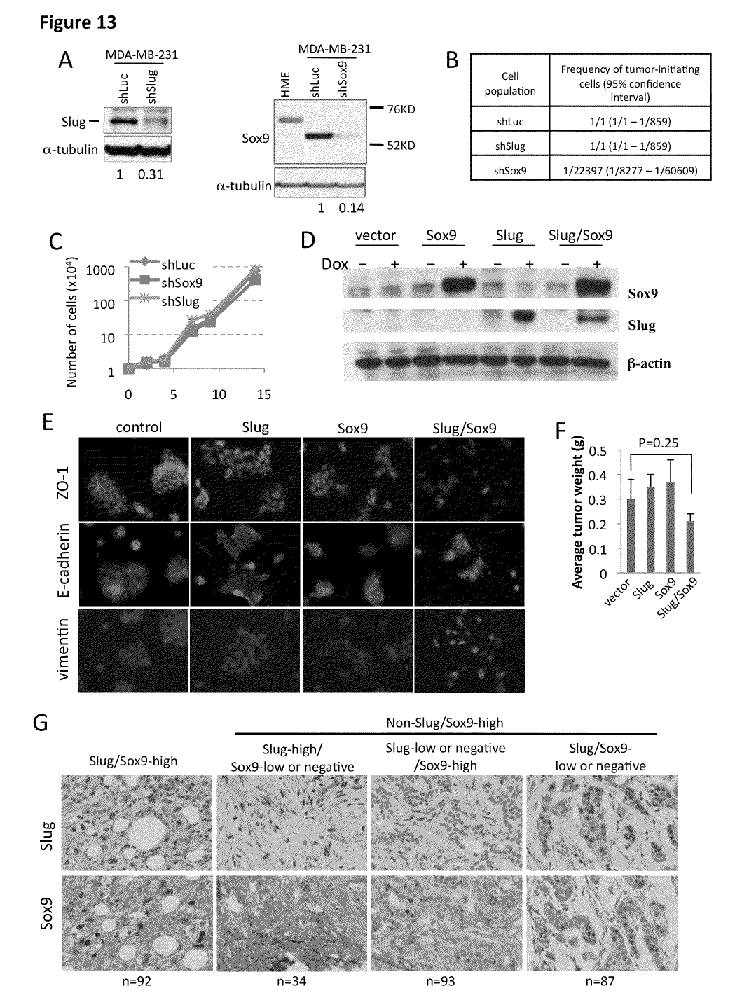

The identification of master regulators of the normal MaSC state in murine MECs, as described above, provided an opportunity to test whether a similar regulatory circuitry operates in human breast CSCs. We first examined whether Slug and Sox9 were required to maintain the tumor-initiating ability of usually aggressive MDA-MB-231 human breast cancer cells. These cells expressed significant levels of the Slug protein and a Sox9 isoform that was ˜10 kDa smaller than the Sox9 protein expressed in normal human MECs (FIG. 13A). The precise nature of this isoform is unknown.

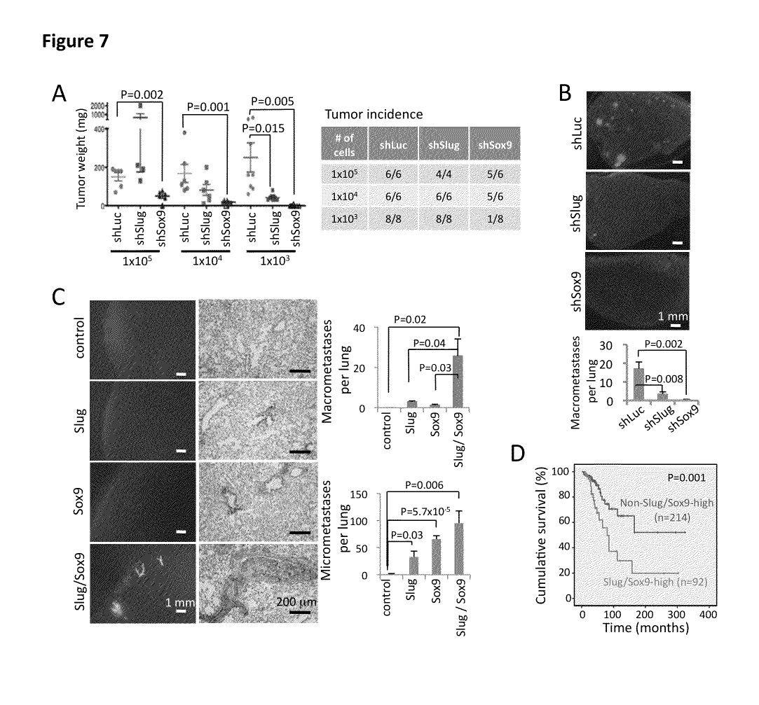

We found that knockdown of either Slug or Sox9 greatly inhibited the tumor-initiating ability of MDA-MB-231 cells. The shSox9-expressing cells had a greater than 70-fold lower tumor-initiating ability than did the control-vector-expressing cells, as calculated by limiting dilution analysis (FIG. 7A and FIG. 13B). Unlike the shSox9-expressing cells, however, the shSlug-expressing cells could form primary tumors at the same frequency as the control-shRNA-expressing cells, but the resulting tumors were 6-fold smaller upon Slug knockdown (FIG. 7A). In contrast to their dramatic effects on tumor growth, shSox9 and shSlug had no adverse effect on the proliferation of MDA-MB-231 cells in monolayer culture (FIG. 13C). These results demonstrated that Sox9 and, to a lesser extent, Slug are required for maintaining the robust tumorigenicity of MDA-MB-231 cells.

During the process of metastasis, tumor-initiating ability would appear to be critical for disseminated cancer cells to seed metastases (Nguyen et al., 2009; Valastyan and Weinberg, 2011). We therefore tested whether knocking down Slug and Sox9 also inhibited the experimental metastasis of MDA-MB-231 cells upon tail vein injection. Consistent with their effects in tumorigenesis, Slug knockdown inhibited the metastasis formation by MDA-MB-231 cells in the lungs by 5-fold, while Sox9 knockdown inhibited metastasis by more than 40-fold (FIG. 7B).

We further tested whether Slug and Sox9 could function cooperatively to induce metastasis-seeding cells in the otherwise-non-metastatic MCF7ras human breast cancer cells. We implanted orthotopically MCF7ras cells transduced with inducible Slug, Sox9 or both TFs in NOD-SCID mice. The mice were then treated with doxycycline for two weeks. At this time point, Slug and Sox9 had induced a clear, although partial EMT in MCF7ras cells (FIG. 13E). (Interestingly, in MCF7ras cells, Sox9 could enhance Slug-induced EMT. This is different from the result in primary mouse MECs, where this did not occur. Without wishing to be bound by any theory, it is likely that Slug alone was already sufficient to induce a near complete EMT in primary murine MECs, explaining why Sox9 was not needed to further enhance the EMT. However, in MCF7ras cells, Slug alone induced only a weak EMT, explaining why Sox9 could further promote the EMT when co-expressed with Slug. This suggests Sox9 has EMT-dependent and -independent functions.) The animals were then kept on a doxycycline-free diet for ten weeks and examined for primary tumor growth and lung metastasis thereafter. The control MCF7ras cells generated virtually no detectable macroscopic metastases (macrometastases) and only a few microscopic metastases (micrometastases) per lung (FIG. 7C). Transient expression of either Slug or Sox9 alone in the primary tumors generated numerous micrometastases per lung, but only led to a few macrometastases. However, when Slug and Sox9 were concomitantly expressed for two weeks in the primary tumors, the number of macrometastases dramatically increased, from virtually no macrometastases in control vector-transduced cells to ˜26 macrometastases per lung in Slug and Sox9-coexpressed cells (FIG. 7C). Hence, the coexpression of Slug and Sox9 induced macrometastasis-seeding cells in usually non-metastatic MCF7ras cells.

We sought to extend our findings in human breast cancer cell lines to clinical samples by examining the expression of Slug and Sox9 proteins in a panel of 306 clinically-defined human breast cancer samples on tissue microarray. We found 92 cases of cancer samples expressing high levels of both Slug and Sox9 and 214 cases expressing high levels of only one factor or neither (FIG. 13G). The patients with primary tumors expressing high levels of both Slug and Sox9 have a significantly lower overall survival than the rest of patients (˜20% vs. ˜50% cumulative overall survival, P<0.0001) (FIG. 7D). These results showed that high expression levels of both Slug and Sox9 in human breast cancers are associated with poor patient outcomes, consistent with the effects of these two factors on promoting tumorigenesis and metastasis.

### EXPERIMENTAL PROCEDURES

Mouse Primary MEC Isolation and FACS

Primary MECs were isolated from mammary glands of 8- to 14-week-old virgin mice by collagenase, dispase and trypsine digestion. Various MEC subpopulations were FACS sorted after staining single cells with antibodies against EpCAM, CD61, CD49f and specific lineage markers. More details are provided below.

Matrigel Organoid Culture

Cells were dissociated into single cells and cultured with Epicult-B medium (Stem Cell Technology) containing 5% Matrigel, 5% heat-inactivated FBS, 10 ng/ml EGF, 20 ng/ml bFGF, 4 μg/ml heparin and 5 μM Y-27632. Cells were seeded at 1000-2000 per well in 96-well ultra-low attachment plates (Corning). All organoid cultures were performed in the absence of doxycycline. The number of organoids (>100 μm in diameter) was counted 7-14 days after the seeding.

Cleared Mammary Fat Pad Transplantation

Cell aliquots resuspended in 10 μl PBS containing 25% Matrigel were injected into inguinal mammary fat pads of NOD-SCID mouse, which had been cleared of endogenous mammary epithelium at 3 weeks old. Details of the reconstitution analysis are provided below.

Statistical Analysis

All data are presented as mean±standard error of mean except specified otherwise. Student t-test was used to calculate P values except in limiting dilution analyses, for which the Extreme Limiting Dilution Analysis Program was used. P<0.05 was considered significant.

Mouse Reagents

Mice ubiquitously expressing rtTA and EGFP in the mammary gland and other tissues were generated by breeding Rosa26-M2rtTA mice (Beard et al., 2006) with CAG-EGFP mice (Jackson Laboratory). CAG-DsRed*MST mice, which express dsRed ubiquitously, were obtained from the Jackson Laboratory. A colony of NOD-SCID mice was maintained in-house.

Slug-YFP mice were generated by targeting an IRES-YFP cassette into the 3′UTR of the Snai2 locus through homologous recombination. A BAC clone containing the Snai2 locus was recombined with the pL253 vector through gap-repair. An IRES-YFP (Venus)-polyA cassette (Nagai et al., 2002) was subcloned into the pL452 vector. These two vectors were recombined to generate a targeting vector containing the IRES-YFP-polyA cassette in the 3′UTR of the Snai2 locus by using a published recombineering technology (Copeland et al., 2001). The targeting vector was linearized and electroporated into F1 hybrid (C57BL/6×129S4Sv/Jae)-derived v6.5 ES cells. The chimeric mice were then bred into the C57Bl/6 background.

Primary MEC Isolation and FACS

Mammary glands were minced and then digested with 1.5 mg/ml collagenase A (Roche) in the DME/F12 medium at 37° C. for 2 hours. The digested samples were washed with PBS and spun down at 800 rpm for 1 minute to enrich for mammary epithelial organoids twice. The organoids were further digested with 0.05% trypsine for 5 minutes and 5 mg/ml dispase (Stem Cell Technology) plus 100 ug/ml DNase (Roche) for 5 minutes, then filtered through 40 μm cell strainers to obtain single cells. For separating various MEC subpopulations, single MECs were stained with antibodies against CD49f (PerCP-Cy5.5, BioLegend), CD29 (PE/Cy7, BioLegend), CD61 (PE), EpCAM (APC) and lineage markers (biotinylated anti-CD45, -CD31 and -Ter-119 primary antibodies plus eFluor™ 450-conjugated streptavidin (BD Bioscience)). All antibodies were from eBioscience except indicated otherwise. The stained cells were sorted on a FACSAria II sorter.

Cell Culture

Primary MECs were cultured in advanced DMEM/F12 (Invitrogen) supplemented with 2% calf serum, 10 ng/ml EGF, 10 ng/ml bFGF (Millipore), 4 μg/ml heparin (Sigma-Aldrich), 5 μM Y-27632 ROCK inhibitor (Tocris) and 0.5 μM Bio GSK-3 inhibitor (Tocris) MDA-MB-231 and MCF7ras cells were culture in DME plus 10% heat-inactivated fetal bovine serum. Cells were treated with 1-2 μg/ml Doxycycline Hyclate (Sigma-Aldrich) to induce tetracycline-inducible gene expression.

Lentiviral Vectors and Infection

Mouse Slug, Snail and Twist1 cDNAs prepared from IMAGE clones (Open Biosystems) or pBP-Twist1 (Yang et al., 2004) were subcloned into the pTK380 tetracycline-inducible lentiviral vector (Haack et al., 2004). Mouse Sox9 and human Sox10 cDNAs obtained from Open Biosystems or Harvard DNA Resource Core were subcloned into the FUW-LPT2 tetracycline-inducible lentiviral vector (modified from FUW-tetO by Kong-Jie Kah). Mouse cDNAs (Sox2, Sox9, Myc, Klf4, FoxD3 and Hes1) and human cDNAs (Sox4 and β-cateninΔN90, a constitutively active β-catenin mutant) were subcloned into the pWPXL lentiviral vector (Addgene). For lentiviral infection, MECs were seeded at 5×10⁴-1×10⁵ cells per 6 cm dish and transduced 24 hours later with concentrated virus in the presence of 5 μg/ml polybrene. The infection efficiency was routinely greater than 80%.

The shRNAs were all cloned in the pLKO.1-puro lentiviral vector. Their sources or targeting sequences are listed as the following:

Mouse shSlug-3—Open Biosystems, RMM3981-99015334

Mouse shSlug-4—Open Biosystems, RMM3981-99015342

Mouse shSox9-2—Open Biosystems, RMM3981-97074461

Mouse shSox9-5—Open Biosystems, RMM3981-97074464

Human shSox9—Open Biosystems, RHS3979-9587792

human shSlug—clone #2 from Gupta et al., 2005

Mouse shSox10-2—GGAGGTTGCTGAACGAAAGTG (SEQ ID NO. 62)

Mouse shTwist2-3—Open Biosystems, TRCN0000086085

Mouse shTwist2-4—Open Biosystems, TRCN0000086086

shLuciferase—CCTAAGGTTAAGTCGCCCTCG (SEQ ID NO. 63)

Cleared Mammary Fat Pad Reconstitution Analysis

Transplanted mammary fat pads were examined for gland reconstitution by whole-mount analyses 6-12 weeks post-injection. Only the presence of branched ductal trees with lobules and/or terminal end buds was scored as a positive reconstitution. For quantifying competitive reconstitution, fat pads containing reconstituted ductal trees were first dissociated into single cells. The ratios of GFP-versus dsRed-positive cells were then measured by flow cytometry. For limiting dilution analyses, the frequency of MaSCs in the cell population being transplanted was calculated using the Extreme Limiting Dilution Analysis Program (http://bioinf.wehi.edu.au/software/elda/index.html) (Hu and Smyth, 2009). For secondary mammary gland reconstitution, primary mammary ductal outgrowths were cut into fragments of 1 mm³ and re-implanted into cleared mammary fat pads at one fragment per implantation.

Tumor Implantation, Metastasis Analysis and In Vivo Doxycycline Treatment

For subcutaneous injections, MDA-MB-231 cells resuspended in 500 PBS containing 25% Matrigel were injected into the flanks of NOD-SCID mice. The tumor incidence and weight were measured three months post-injection. For experimental metastasis experiments, 1×10⁶ GFP-labeled MDA-MB-231 cells resuspended in 100ul PBS were injected into each NOD-SCID mouse through the tail vein. The lungs were examined for metastases three months after injection. For orthotopic tumor transplantations, MCF7ras cells labeled with the tdTomato fluorescent protein were resuspended in 15 μl Matrigel and injected into mammary fat pads of NOD-SCID mice. For metastasis quantification, lungs were examined under a Leica fluorescent dissecting microscope. Metastases detectable at 12× magnification were scored as macro-metastases, and at 33× were scored as micro-metastases. The in vivo doxycycline treatment was administered through drinking water containing 2 mg/ml doxycycline and 10 mg/ml sucrose.

Immunofluorescence and Western Blot

Formalin-fixed paraffin-embedded or fresh-frozen OCT-embedded tissue sections or methanol-fixed cells were stained with antibodies against Slug (Cell Signaling Technology, #9585), Sox9 (R&D AF3075 or Millipore AB5535), cytokeratin 8 (Developmental Studies Hybridoma Bank, clone Tromal), cytokeratin 14 (Covance, PRB-155P), milk proteins (Nordic Immunology, RAM/TM), α-SMA (Sigma, A5691), E-cadherin (BD Transduction, 610181), ZO-1 (Invitrogen, 40-2200), and vimentin (BD Transduction 550513). Immunoblotting was performed with antibodies against E-cadherin, N-cadherin, Vimentin (all from BD Transduction), Slug (Cell Signaling, #9585), Sox9 (Millipore, AB5535), α-tubulin (Abeam), and β-actin (Abeam).

Quantitative RT-PCR

Total RNA was isolated either directly from cultured cells or from cells treated with RNA later (Ambion) using the RNA Easy Miniprep Kit (Qiagen) and reverse transcribed using the High Capacity RNA-to-cDNA Kit (Applied Biosystems). Real-time PCR was performed using SYBR Green I master mix (Roche) on a LightCycler 480 instrument (Roche). Real-time PCR primer sequences were listed in Table S

TABLE S1
Real-time PCR primer sequences.

| Mouse Genes | Forward Primers | Reverse Primers |
|---|---|---|
| Snail | AAGATGCACATCCGAAGCCA | CTCTTGGTGCTTGTGGAGCA |
| | (SEQ ID NO. 1) | (SEQ ID NO. 2) |
| Slug (CDS) | CTCACCTCGGGAGCATACAG | GACTTACACGCCCCAAGGATG |
| | (SEQ ID NO. 3) | (SEQ ID NO. 4) |
| Slug (5′UTR) | GAGCCGGGTGACTTCAGAG | GGCGTTGAAATGTTTCTTGA |
| | (SEQ ID NO. 5) | (SEQ ID NO. 6) |
| Twist1 | CTGCCCTCGGACAAGCTGAG | CTAGTGGGACGCGGACATGG |
| | (SEQ ID NO. 7) | (SEQ ID NO. 8) |
| Twist2 | CGCTACAGCAAGAAATCGAGC | GCTGAGCTTGTCAGAGGGG |
| | (SEQ ID NO. 9) | (SEQ ID NO. 10) |
| Zeb1 | GTTCTGCCAACAGTTGGTTT | GCTCAAGACTGTAGTTGATG |
| | (SEQ ID NO. 11) | (SEQ ID NO. 12) |
| Zeb2 | TCTGAAGATGAAGAAGGCTG | AGTGAATGAGCCTCAGGTAA |
| | (SEQ ID NO. 13) | (SEQ ID NO. 14) |
| FoxC2 | TCCTGGTATCTGAACCACGG | TCAGTATTTGGTGCAGTCGT |
| | (SEQ ID NO. 15) | (SEQ ID NO. 16) |
| Goosecoid | TCTCAACCAGCTGCACTGTC | GGTCTGGTTTAAGAACCGCC |
| | (SEQ ID NO. 17) | (SEQ ID NO. 18) |
| TCF3 | CTCGATCTACTCCCCGGATC | CCAGTGACATGGGGCCGGTG |
| | (SEQ ID NO. 19) | (SEQ ID NO. 20) |
| Klf8 | TCAGAAAGTGGTTCGATGCAG | AACAGAGCTGGGTTCTCCATT |
| | (SEQ ID NO. 21) | (SEQ ID NO. 22) |
| p63ΔN | CCTGGAAAACAATGCCCAGAC | GAGGAGCCGTTCTGAATCTGC |
| | (SEQ ID NO. 23) | (SEQ ID NO. 24) |
| ID4 | CAGTGCGATATGAACGACTGC | GACTTTCTTGTTGGGCGGGAT |
| | (SEQ ID NO. 25) | (SEQ ID NO. 26) |
| Egr2 | GCCAAGGCCGTAGACAAAATC | CCACTCCGTTCATCTGGTCA |
| | (SEQ ID NO. 27) | (SEQ ID NO. 28) |
| Mef2C | TGCTGGTCTCACCTGGTAAC | ATCCTTTGATTCACTGATGGCAT |
| | (SEQ ID NO. 29) | (SEQ ID NO. 30) |
| Tbx2 | CCGATGACTGCCGCTATAAGT | CCATCCACTGTTCCCCTGT |
| | (SEQ ID NO. 31) | (SEQ ID NO. 32) |
| c-Kit | GCCACGTCTCAGCCATCTG | GTCGCCAGCTTCAACTATTAACT |
| | (SEQ ID NO. 33) | (SEQ ID NO. 34) |
| Elf5 | ATGTTGGACTCCGTAACCCAT | GCAGGGTAGTAGTCTTCATTGCT |
| | (SEQ ID NO. 35) | (SEQ ID NO. 36) |
| Cxcr4 | GAAGTGGGGTCTGGAGACTAT | TTGCCGACTATGCCAGTCAAG |
| | (SEQ ID NO. 37) | (SEQ ID NO. 38) |
| LBP | CCTGAGACTCGCCATCTCTGA | AGGAGGAGGTCCACTGAAATG |
| | (SEQ ID NO. 39) | (SEQ ID NO. 40) |
| Sox9-CDS | GAGCCGGATCTGAAGAGGGA | GCTTGACGTGTGGCTTGTTC |
| | (SEQ ID NO. 41) | (SEQ ID NO. 42) |
| Sox9-5′UTR | GGGAGCGACAACTTTACCAG | AGGAGGGAGGGAAAACAGAG |
| | (SEQ ID NO. 43) | (SEQ ID NO. 44) |
| Sox10 | CCCACACTACACCGACCAG | GGCCATAATAGGGTCCTGAGG |
| | (SEQ ID NO. 45) | (SEQ ID NO. 46) |
| Cldn1 | GGGGACAACATCGTGACCG | AGGAGTCGAAGACTTTGCACT |
| | (SEQ ID NO. 47) | (SEQ ID NO. 48) |
| Cldn3 | ACCAACTGCGTACAAGACGAG | CAGAGCCGCCAACAGGAAA |
| | (SEQ ID NO. 49) | (SEQ ID NO. 50) |
| Cldn4 | GTCCTGGGAATCTCCTTGGC | TCTGTGCCGTGACGATGTTG |
| | (SEQ ID NO. 51) | (SEQ ID NO. 52) |
| N-cadherin | ATGTGCCGGATAGCGGGAGC | TACACCGTGCCGTCCTCGTC |
| | (SEQ ID NO. 53) | (SEQ ID NO. 54) |
| E-cadherin | CACCTGGAGAGAGGCCATGT | TGGGAAACATGAGCAGCTCT |
| | (SEQ ID NO. 55) | (SEQ ID NO. 56) |
| Vimentin | CTTGAACGGAAAGTGGAATCCT | GTCAGGCTTGGAAACGTCC |
| | (SEQ ID NO. 57) | (SEQ ID NO. 58) |
| GAPDH | CGTATTGGGCGCCTGGTCAC | ATGATGACCCTTTTGGCTCC |
| | (SEQ ID NO. 59) | (SEQ ID NO. 60) |

Single-Molecule FISH

Single-molecule FISH was performed as published (Raj et al., 2008). We used probe libraries consisting of 48 and 40 20-bp oligonucleotide probes complementary to the coding sequences of Sox9 and Slug, respectively. Sox9 probes were labeled with Alexa594 fluorophores, and Slug probes were labeled with cy5 fluorophores. Co-hybridizations were performed overnight on 6 μm cryo-sections. An additional FITC conjugated antibody against E-cadherin (BD Biosciences) was added to the hybridization mix, and the DAPI dye for nuclear staining was added during the washes. The E-cadherin and nucleus fluorescence was used to assist in segmenting individual cells. Images were taken with a Nikon TE2000 inverted fluorescence microscope equipped with a 100× oil-immersion objective and a Princeton Instruments camera using MetaMorph software (Molecular Devices, Downington, Pa.). The image-plane pixel dimension was 0.13 microns. Quantification was done on 5-12 stacks with Z-spacing of 0.3 microns, in which no more than a single cell was observed. Transcript concentrations were determined by dividing the number of transcripts per cell by the cell volume.

Correlation Analysis of Slug/Sox9 Expression and Patient Outcome

Formalin-fixed paraffin-embedded tumor tissues of 306 breast cancer patients with primary breast cancer were assembled on a tissue microarray (TMA). The tissue collection consisted of 132 cases of pT1 (43.1%), 134 pT2 (43.8%), 21 pT3 (6.9%), 19 pT4 (6.2%); 92 pN0 (34.1%), 136 pN1 (50.4%), 22 pN2 (8.1%), 20 pN3 (7.4%); 41 G1 (114%), 144 G2 (47.1%), 121 G3 (39.5%). For 36 patients, pN category was not available. Age range was from 22-91 years (mean age 58 years). Mean duration of follow-up was 40 months (range 4-324 months) for overall survival. The project was approved by the ethical committee of the Kanton of Zurich (reference number StV-12-2005).

Immunohistochemistry (IHC) of TMA sections was performed using the following primary antibodies: anti-Slug mAb (Cell Signaling Technology, C19G7, 1:100) and anti-Sox9 (Millipore, AB5535, 1:400). IHC stains for Slug and Sox9 were homogenous across entire tumor areas, as tested by whole tumor sections. IHC stains were appraised as positive (≧5% positive cells, scored 1 for weak expression, 2 for moderate expression and 3 for strong expression) or negative (scored 0, <5% positive cells). Samples with scores above the median were classified as “high”, and samples scored below median were classified as “low/negative”. For overall survival analysis, samples scored high for both Slug and Sox9 expression were compared with all the rest samples. Correlations with overall survival were determined by the Kaplan-Meier method using log rank tests. Statistical analyzes were performed using PASW, version 18.0. P-values <0.05 were considered significant.

### REFERENCES

Asselin-Labat, M. L., Sutherland, K. D., Barker, H., Thomas, R., Shackleton, M., Forrest, N. C., Hartley, L., Robb, L., Grosveld, F. G., van der Wees, J., et al. (2007). Gata-3 is an essential regulator of mammary-gland morphogenesis and luminal-cell differentiation. Nat Cell Biol 9, 201-209.
Boyer, L. A., Lee, T. I., Cole, M. F., Johnstone, S. F., Levine, S. S., Zucker, J. P., Guenther, M. G., Kumar, R. M., Murray, H. L., Jenner, R. G., et al. (2005). Core transcriptional regulatory circuitry in human embryonic stem cells. Cell 122, 947-956.
Chaffer, C. L., Brueckmann, I., Scheel, C., Kaestli, A. J., Wiggins, P. A., Rodrigues, L. O., Brooks, M., Reinhardt, F., Su, Y., Polyak, K., et al. (2011). Normal and neoplastic nonstem cells can spontaneously convert to a stem-like state. Proc Natl Acad Sci USA 108, 7950-7955.
Chen, X., Xu, H., Yuan, P., Fang, F., Huss, M., Vega, V. B., Wong, E., Orlov, Y. L., Zhang, W., Jiang, j., et al. (2008). Integration of external signaling pathways with the core transcriptional network in embryonic stem cells. Cell 133, 1106-1117.
Cheung, M., Chaboissier, M. C., Mynett, A., Hirst, E., Schedl, A., and Briscoe, J. (2005). The transcriptional control of trunk neural crest induction, survival, and delamination. Dev Cell 8, 179-192.
DeOme, K. B., Faulkin, L. J., Jr., Bern, H. A., and Blair, P. B. (1959). Development of mammary tumors from hyperplastic alveolar nodules transplanted into gland-free mammary fat pads of female C3H mice. Cancer Res 19, 515-520.
Dick, J. E. (2008). Stem cell concepts renew cancer research. Blood 112, 4793-4807.
Furuyama, K., Kawaguchi, Y., Akiyama, H., Horiguchi, M., Kodama, S., Kuhara, T., Hosokawa, S., Elbahrawy, A., Soeda, T., Koizumi, M., et al. (2011). Continuous cell supply from a Sox9-expressing progenitor zone in adult liver, exocrine pancreas and intestine, Nat Genet 43, 34-41.
Gupta, P. B., Fillmore, C. M., Jiang, G., Shapira, S. D., Tao, K., Kuperwasser, C., and Lander, E. S. (2011). Stochastic state transitions give rise to phenotypic equilibrium in populations of cancer cells. Cell 146, 633-644.
Halder, G., Callaerts, P., and Gehring, W. J. (1995). Induction of ectopic eyes by targeted expression of the eyeless gene in Drosophila. Science 267, 1788-1792.
Huber, M. A., Kraut, N., and Beug, H. (2005). Molecular requirements for epithelial-mesenchymal transition during tumor progression. Curr Opin Cell Biol 17, 548-558.
Kim, J., Chu, J., Shen, X., Wang, J., and Orkin, S. H. (2008). An extended transcriptional network for pluripotency of embryonic stem cells. Cell 132, 1049-1061.
Kopp, J. L., Dubois, C. L., Schaffer, A. E., Hao, E., Shih, H. P., Seymour, P. A., Ma, J., and Sander, M. (2011). Sox9+ ductal cells are multipotent progenitors throughout development but do not produce new endocrine cells in the normal or injured adult pancreas. Development 138, 653-665.
Kordon, E. C., and Smith, G. H. (1998). An entire functional mammary gland may comprise the progeny from a single cell. Development 125, 1921-1930.
Lessard, J., and Sauvageau, G. (2003). Bmi-1 determines the proliferative capacity of normal and leukaemic stem cells. Nature 423, 255-260.
Lim, E., Vaillant, F., Wu, D., Forrest, N. C., Pal, B., Hart, A. H., Asselin-Labat, M. L., Gyorki, D. E., Ward, T., Partanen, A., et al. (2009). Aberrant luminal progenitors as the candidate target population for basal tumor development in BRCA1 mutation carriers. Nat Med 15, 907-913.
Lim, E., Wu, D., Pal, B., Bouras, T., Asselin-Labat, M. L., Valliant, F., Yagita, H., Lindeman, G. J., Smyth, G. K., and Visvader, J. E. (2010). Transcriptome analyses of mouse and human mammary cell subpopulations reveal multiple conserved genes and pathways. Breast Cancer Res 12, R21.
Lobo, N. A., Shimono, Y., Qian, D., and Clarke, M. F. (2007). The biology of cancer stem cells. Annu Rev Cell Dev Biol 23, 675-699.
Mani, S. A., Guo, W., Liao, M. J., Eaton, E. N., Ayyanan, A., Zhou, A. Y., Brooks, M., Reinhard, F., Zhang, C. C., Shipitsin, M., et al. (2008). The epithelial-mesenchymal transition generates cells with properties of stem cells. Cell 133, 704-715.
Morel, A. P., Lievre, M., Thomas, C., Hinkal, G., Ansieau, S., and Puisieux, A. (2008). Generation of breast cancer stem cells through epithelial-mesenchymal transition. PLoS ONE 3, e2888.
Nguyen, D. X., Bos, P. D., and Massague, J. (2009). Metastasis: from dissemination to organ-specific colonization. Nat Rev Cancer 9, 274-284.
Nowak, J. A., Polak, L., Pasolli, H. A., and Fuchs, E. (2008). Hair follicle stem cells are specified and function in early skin morphogenesis. Cell Stem Cell 3, 33-43.
Pece, S., Tosoni, D., Confalonieri, S., Mazzarol, G., Vecchi, M., Ronzoni, S., Bernard, L., Viale, G., Pelicci, P. G., and Di Fiore, P. P. (2010). Biological and molecular heterogeneity of breast cancers correlates with their cancer stem cell content. Cell 140, 62-73.
Proia, T. A., Keller, P. J., Gupta, P. B., Klebba, I., Jones, A. D., Sedic, M., Gilmore, H., Tung, N., Naber, S. P., Schnitt, S., et al. (2011). Genetic predisposition directs breast cancer phenotype by dictating progenitor cell fate. Cell Stem Cell 8, 149-163.
Reya, T., Morrison, S. J., Clarke, M. F., and Weissman, I. L. (2001). Stem cells, cancer, and cancer stem cells. Nature 414, 105-111.
Sato, T., Vries, R. G., Snippert, H. J., van de Wetering, M., Barker, N., Stange, D. E., van Es, J. H., Abo, A., Kujala, P., Peters, P. J., et al. (2009). Single Lgr5 stem cells build crypt-villus structures in vitro without a mesenchymal niche. Nature 459, 262-265.
Scheel, C., Eaton, E. N., Li, S. H., Chaffer, C. L., Reinhardt, F., Kah, K. J., Bell, G., Guo, W., Rubin, J., Richardson, A. L., et al. (2011). Paracrine and autocrine signals induce and maintain mesenchymal and stem cell states in the breast. Cell 145, 926-940.
Shackleton, M., Vaillant, F., Simpson, K. J., Stingl, J., Smyth, G. K., Asselin-Labat, M. L., Wu, L., Lindeman, G. J., and Visvader, J. E. (2006). Generation of a functional mammary gland from a single stem cell. Nature 439, 84-88.
Shimono, Y., Zabala, M., Cho, R. W., Lobo, N., Dalerba, P., Qian, D., Diehn, M., Liu, H., Panula, S. P., Chiao, E., et al. (2009). Downregulation of miRNA-200c links breast cancer stem cells with normal stem cells. Cell 138, 592-603.
Stingl, J., Eirew, P., Ricketson, I., Shackleton, M., Vaillant, F., Choi, D., Li, H. I., and Eaves, C. J. (2006a). Purification and unique properties of mammary epithelial stem cells. Nature.
Stingl, J., Raouf, A., Eirew, P., and Eaves, C. J. (2006b). Deciphering the mammary epithelial cell hierarchy. Cell Cycle 5, 1519-1522.
Takahashi, K., and Yamanaka, S. (2006). Induction of pluripotent stem cells from mouse embryonic and adult fibroblast cultures by defined factors. Cell 126, 663-676.
Tapscott, S. J., Davis, R. L., Thayer, M. J., Cheng, P. F., Weintraub, H., and Lassar, A. B. (1988). MyoD1: a nuclear phosphoprotein requiring a Myc homology region to convert fibroblasts to myoblasts. Science 242, 405-411.
Thiery, J. P., Acloque, H., Huang, R. Y., and Nieto, M. A. (2009). Epithelial-mesenchymal transitions in development and disease. Cell 139, 871-890.
Valastyan, S., and Weinberg, R. A. (2011). Tumor metastasis: molecular insights and evolving paradigms. Cell 147, 275-292.
van der Flier, L. G., van Gijn, M. E., Hatzis, P., Kujala, P., Haegebarth, A., Stange, D. E., Begthel, H., van den Born, M., Guryev, V., Oving, I., et al. (2009). Transcription factor achaete scute-like 2 controls intestinal stem cell fate. Cell 136, 903-912.
Vidal, V. P., Chaboissier, M. C., Lutzkendorf, S., Cotsarelis, G., Mill, P., Hui, C. C., Ortonne, N., Ortonne, J. P., and Schedl, A. (2005). Sox9 is essential for outer root sheath differentiation and the formation of the hair stem cell compartment. Curr Biol 15, 1340-1351.
Visvader, J. E. (2009). Keeping abreast of the mammary epithelial hierarchy and breast tumorigenesis. Genes Dev 23, 2563-2577.
Watanabe, K., Ueno, M., Kamiya, D., Nishiyama, A., Matsumura, M., Wataya, T., Takahashi, J. B., Nishikawa, S., Muguruma, K., and Sasai, Y. (2007). A ROCK inhibitor permits survival of dissociated human embryonic stem cells. Nat Biotechnol 25, 681-686.
Zhao, C., Blum, J., Chen, A., Kwon, H. Y., Jung, S. H., Cook, J. M., Lagoo, A., and Reya, T. (2007). Loss of beta-catenin impairs the renewal of normal and CML stem cells in vivo. Cancer Cell 12, 528-541.

Those skilled in the art will recognize, or be able to ascertain using no more than routine experimentation, many equivalents to the specific embodiments described herein. The scope of the present invention is not intended to be limited to the Description or the details set forth therein.

Section headings used herein are not to be construed as limiting in any way. It is contemplated that subject matter presented under any section heading may be applicable to, or combined with, any aspect or embodiment described herein.

Embodiments or aspects herein may be directed to any agent, composition, article, kit, and/or method described herein. It is contemplated that any one or more embodiments or aspects can be freely combined with any one or more other embodiments or aspects whenever appropriate. For example, any combination of two or more agents, compositions, articles, kits, and/or methods that are not mutually inconsistent, is provided. Where the phrase “in some embodiments” is used herein, it should be understood that embodiments pertaining to any relevant aspect described herein are provided.

Articles such as “a”, “an” and “the” may mean one or more than one unless indicated to the contrary or otherwise evident from the context. Claims or descriptions that include “or” between one or more members of a group are considered satisfied if one, more than one, or all of the group members are present in, employed in, or otherwise relevant to a given product or process unless indicated to the contrary or otherwise evident from the context. The embodiments in which exactly one member of the group is present in, employed in, or otherwise relevant to a given product or process, are provided. Embodiments in which more than one, or all of the group members are present in, employed in, or otherwise relevant to a given product or process, are provided. It is contemplated that all embodiments described herein are applicable to all different aspects described herein, wherever appropriate. It is also contemplated that any of the embodiments can be freely combined with one or more other such embodiments whenever appropriate. Furthermore, it is to be understood that all variations, combinations, and permutations in which one or more limitations, elements, clauses, descriptive terms, etc., from one or more of the claims (whether original or subsequently added claims) is introduced into another claim (whether original or subsequently added), are provided. For example, any claim that is dependent on another claim can be modified to include one or more elements or limitations found in any other claim that is dependent on the same base claim, and any claim that refers to an element present in a different claim can be modified to include one or more elements or limitations found in any other claim that is dependent on the same base claim as such claim. References to a “claim” should be considered to apply to such claim as existing when filed and/or following any amendment thereto. Furthermore, where claims or other description herein recite a composition, methods of making the composition, e.g., according to methods disclosed herein, and methods of using the composition, e.g., for purposes disclosed herein, are provided. Where the claims or other description herein recite a method, compositions suitable for performing the method, and methods of making the composition, are provided. Where the claims or other description herein recite a method of making a composition, compositions made according to the methods and methods of using the composition, are provided, unless otherwise indicated or unless one of ordinary skill in the art would recognize that a contradiction or inconsistency would arise.

Where elements are presented as lists, e.g., in Markush group format, each subgroup of the elements is also disclosed, and any element(s) can be removed from the group. For purposes of conciseness only some of these embodiments may have been specifically recited herein, but all such embodiments are encompassed. It should also be understood that, in general, where aspects or embodiments, is/are referred to as comprising particular elements, features, etc., certain aspects or embodiments consist, or consist essentially of, such elements, features, etc.

Where numerical ranges are mentioned herein, embodiments in which the endpoints are included, embodiments in which both endpoints are excluded, and embodiments in which one endpoint is included and the other is excluded, are provided. It should be assumed that both endpoints are included unless indicated otherwise. Furthermore, unless otherwise indicated or otherwise evident from the context and understanding of one of ordinary skill in the art, values that are expressed as ranges can assume any specific value or subrange within the stated ranges in various embodiments, to the tenth of the unit of the lower limit of the range, unless the context clearly dictates otherwise. Where phrases such as “less than X”, “greater than X”, or “at least X” is used (where X is a number or percentage), it should be understood that any reasonable value can be selected as the lower or upper limit of the range. It is also understood that where a list of numerical values is stated herein (whether or not prefaced by “at least”), embodiments that relate to any intervening value or range defined by any two values in the list are provided, and that the lowest value may be taken as a minimum and the greatest value may be taken as a maximum. Furthermore, where a list of numbers, e.g., percentages, is prefaced by “at least”, the term applies to each number in the list. For any embodiment in which a numerical value is prefaced by “about” or “approximately”, embodiments in which the exact value is recited are provided. For any embodiment in which a numerical value is not prefaced by “about” or “approximately”, the embodiments in which the value is prefaced by “about” or “approximately” are provided. “Approximately” or “about” generally includes numbers that fall within a range of 1% or in some embodiments 5% or in some embodiments 10% of a number in either direction (greater than or less than the number) unless otherwise stated or otherwise evident from the context (e.g., where such number would impermissibly exceed 100% of a possible value).

Any particular embodiment(s), aspect(s), element(s), feature(s), etc., e.g., any agent, cell type, condition, disease, etc., or any combination of any of the foregoing, may be explicitly recited in, encompassed by, or excluded from any one or more claims. Any scope of variants and/or specific sequences or level of identity can be recited in, encompassed by, or excluded from any one or more claims.

We claim:

1. A method of generating stem cells from epithelial cells comprising steps of: (a) providing a population of epithelial cells; and (b) inducing epithelial-mesenchymal transition (EMT) and increasing the amount or activity of at least one EMT-cooperating protein in the population of epithelial cells, thereby generating stem cells in the population.

2. The method of claim 1, wherein the EMT-cooperating protein is a transcription factor (TF).

3. The method of claim 1, wherein the EMT-cooperating protein is a Sox protein.

4. The method of claim 1, wherein the EMT-cooperating protein is Sox 9 or Sox10.

5. The method of claim 1, wherein said inducing comprises exposing the population of epithelial cells to an EMT-inducing agent.

6. The method of claim 1, wherein said inducing comprises exposing the population of epithelial cells to an agent that comprises, encodes, or increases expression or activity of a polypeptide comprising an EMT-TF.

7. The method of claim 1, wherein said inducing comprises exposing the population of epithelial cells to an agent that comprises, encodes, or increases expression or activity of a polypeptide comprising an EMT-TF selected from Slug, Snail, Twist1, Twist2, Zeb1, Zeb2, Goosecoid, FoxC2, Tcf3, Klf8, FoxC1, FoxQ1, Six1, Lbx1, Yap1, HIF1, or a functional variant of any of these or exposing the population of epithelial cells to an agent that comprises, encodes, or increases expression or activity of a polypeptide comprising Taz or a functional variant thereof.

8. The method of claim 1, wherein the method comprises exposing the population of epithelial cells to an agent that stimulates TGF-beta, Wnt, Notch, Sonic Hedgehog or EGF pathway signaling.

9. The method of claim 1, wherein said inducing comprises exposing the population of epithelial cells to an agent that comprises a TGF-beta receptor agonist, Wnt receptor agonist, or EGF receptor agonist.

10. The method of claim 1, wherein said increasing comprises exposing the population of epithelial cells to agent that comprises, encodes, or induces expression of a polypeptide comprising an EMT-cooperating protein.

11. The method of claim 1, wherein the method comprises exposing the population of epithelial cells to an EMT-inducing agent and an agent that comprises, encodes, or induces expression of a polypeptide comprising an EMT-cooperating protein.

12. The method of claim 1, wherein said inducing comprises exposing the population of epithelial cells to an EMT-inducing agent and an agent that comprises, encodes, or induces expression of an EMT-cooperating TF.

13. The method of any of claims 5-12, wherein said exposing is transient.

14. The method of claim 1, wherein said inducing comprises introducing a nucleic acid that encodes a polypeptide comprising an EMT-TF into the epithelial cells or inducing expression of a previously introduced nucleic acid that encodes a polypeptide comprising an EMT-TF.

15. The method of claim 1, wherein said increasing comprises introducing a non-integrating nucleic acid that encodes a polypeptide comprising an EMT-cooperating protein into the epithelial cells.

16. The method of claim 1, wherein said inducing and said increasing do not comprise altering the genome of the epithelial cells.

17. The method of claim 1, wherein the population of epithelial cells comprises differentiated epithelial cells and the method comprises generating stem cells from said differentiated epithelial cells.

18. The method of claim 1, wherein the population of epithelial cells comprises luminal epithelial cells and the method comprises generating stem cells from said luminal epithelial cells.

19. The method of claim 1, wherein the population of epithelial cells comprises differentiated luminal epithelial cells and the method comprises generating stem cells from said differentiated luminal epithelial cells.

20. The method of claim 1, wherein the epithelial cells comprise mammary epithelial cells.

21. The method of claim 1, wherein the epithelial cells comprise primary epithelial cells.

22. The method of claim 1, wherein the stem cells comprise cells capable of giving rise to organoids.

23. The method of claim 1, further comprising (c) assessing formation of stem cells in the population.

24. The method of claim 1, further comprising (c) isolating stem cells from the population.

25. The method of claim 1, further comprising (c) administering at least some of the stem cells to a subject.

26. The method of claim 1, further comprising (c) inducing at least some of the stem cells to enter into a more differentiated state.

27. The method of claim 1, further comprising (c) inducing at least some of the stem cells to enter into a more differentiated state; and (d) administering at least some of the resulting cells to a subject.

28. A method of preparing isolated stem cells from epithelial cells, the method comprising the steps of: (a) generating stem cells from epithelial cells according the method of claim 1; and (b) isolating stem cells from the population.

29. A method of converting a cell to a less differentiated state, the method comprising: (a) providing a cell; and (b) increasing the amount or activity of at least one EMT-cooperating TF in the differentiated cell, thereby converting the cell to a less differentiated state.

30. The method of claim 29, wherein the cell is a differentiated epithelial cell.

31. The method of claim 29, further comprising inducing EMT in the cell.

32. A method of generating stem cells, the method comprising: (a) providing a population of cells that express an EMT-TF; and (b) contacting the cells with an agent that increases the amount or activity of at least one EMT-cooperating TF.

33. The method of claim 32, wherein the EMT-cooperating TF comprises a Sox protein or a functional variant thereof.

34. The method of claim 32, wherein the EMT-TF comprises a Slug or Snail protein or a functional variant of either.

35. The method of claim 32, wherein the cells of step (a) endogenously express the EMT-TF.

36. The method of claim 32, wherein the cells of step (a) ectopically express the EMT-TF.

37. The method of claim 32, further comprising inducing at least some of the stem cells to differentiate.

38. A method of generating stem cells, the method comprising: (a) providing a population of cells that express an EMT-cooperating TF; and (b) contacting the cells with an agent that increases the amount or activity of at least one EMT-TF.

39. The method of claim 38, wherein the EMT-TF comprises Slug, Snail or a functional variant of either.

40. The method of claim 38, wherein the EMT-cooperating TF comprises a Sox protein or a functional variant thereof.

41. The method of claim 38, wherein the cells of step (a) ectopically express the EMT-TF.

42. The method of claim 38, wherein the cells of step (a) endogenously express the EMT-TF.

43. The method of claim 38, further comprising inducing at least some of the stem cells to differentiate.

44. An isolated composition or kit comprising: (a) an EMT-inducing agent; and (b) an EMT-cooperating agent.

45. The isolated composition or kit of claim 44, wherein the EMT-cooperating agent comprises, encodes, or induces expression of a polypeptide comprising an EMT-cooperating TF or enhances activity of a polypeptide comprising an EMT-cooperating TF.

46. The isolated composition or kit of claim 44, wherein the EMT-cooperating TF is a Sox protein or a functional variant thereof.

47. The isolated composition or kit of claim 44, wherein the EMT-cooperating TF is Sox 9 or Sox 10.

48. The isolated composition or kit of claim 44, wherein the EMT-inducing agent comprises, encodes, or induces expression of a polypeptide comprising an EMT-TF.

49. The isolated composition or kit of claim 44, wherein the agent that induces EMT comprises one or more: (a) agents that stimulate TGF-beta pathway signaling; (b) agents that inhibit cell adhesion; (c) agents that stimulate Wnt pathway signaling.

50. The isolated composition of claim 44, further comprising epithelial cells.

51. The isolated composition of claim 44, further comprising mammary epithelial cells.

52. A method of generating stem cells from epithelial cells, comprising steps of (a) providing a population of epithelial cells; and (b) contacting the cells with the isolated composition of any of claims 44-49.

53. An isolated epithelial cell comprising an exogenously introduced EMT-inducing agent and an exogenously introduced EMT-cooperating agent.

54. The isolated epithelial cell of claim 53, wherein the exogenously introduced EMT-inducing agent comprises, encodes, or induces expression of a polypeptide comprising an EMT-TF.

55. The isolated epithelial cell of claim 53, wherein the exogenously introduced EMT-cooperating agent comprises, encodes, or induces expression of a polypeptide comprising an EMT-cooperating TF.

56. The isolated epithelial cell of claim 53, wherein the exogenously introduced EMT-cooperating agent comprises, encodes, or induces expression of a polypeptide comprising an EMT-cooperating TF comprising a Sox protein or a functional variant thereof.

57. The isolated epithelial cell of claim 53, wherein the exogenously introduced EMT-cooperating agent comprises, encodes, or induces expression of a polypeptide comprising an EMT-cooperating TF comprising Sox9 or Sox10 or a functional variant of either.

58. The isolated epithelial cell of claim 53, wherein the exogenously introduced EMT-inducing agent comprises, encodes, or induces expression of a polypeptide comprising an EMT-TF comprising Slug, Snail, or a functional variant of either.

59. An isolated epithelial cell comprising a first exogenous nucleic acid that encodes a first polypeptide comprising an EMT-TF and a second exogenous nucleic acid that encodes a second polypeptide comprising an EMT-cooperating TF.

60. The isolated epithelial cell of claim 53, wherein the EMT-TF is Slug, Snail, or a functional variant of either.

61. The isolated epithelial cell of claim 53, wherein the EMT-cooperating TF is a Sox protein or functional variant thereof.

62. The isolated epithelial cell of claim 53, wherein the EMT-TF is Slug or a functional variant thereof and the EMT-cooperating TF is Sox9 or Sox10 or a functional variant of either.

63. The isolated epithelial cell of claim 53, wherein the first and second nucleic acids are not integrated into the genome of the cell.

64. The isolated epithelial cell of claim 53, wherein the cell ectopically expresses the first and second polypeptides.

65. The isolated epithelial cell of claim 53, wherein the cell expresses endogenous counterparts of the EMT-TF and the EMT-cooperating TF.

66. The isolated epithelial cell of claim 53, wherein the cell does not express endogenous counterparts of the EMT-TF and the EMT-cooperating TF.

67. The isolated epithelial cell of claim 53, wherein the cell is a non-tumor cell.

68. The isolated epithelial cell of claim 53, wherein the cell is a tumor cell.

69. The isolated epithelial cell of claim 53, wherein the epithelial cell is a mammary epithelial cell.

70. A method of generating a stem cell, the method comprising: culturing a population of isolated epithelial cells of claim 53 under conditions in which the cells express the first and second polypeptides.

71. The method of claim 70, comprising maintaining the population of cells for a sufficient period of time to induce expression of the endogenous counterparts of the EMT-TF and the EMT-cooperating TF in the population of cells.

72. The method of claim 70, comprising maintaining the population of cells for a sufficient period of time of induce expression of the endogenous counterparts of the EMT-TF and the EMT-cooperating TF in the population of cells; and isolating a stem cell from the population.

73. A method of inhibiting tumor-initiating or metastatic ability of a tumor cell the method comprising: contacting the tumor cell with (a) an EMT inhibitor; and (b) an inhibitor of an EMT-cooperating TF.

74. The method of claim 73, wherein the EMT inhibitor inhibits an EMT-TF.

75. The method of claim 73, wherein the EMT-cooperating TF is a Sox protein.

76. The method of claim 73, wherein the tumor cell expresses high levels of an EMT-inducing TF and the EMT-cooperating protein.

77. A method of treating a subject in need of treatment of a tumor, the method comprising: administering to the subject (a) an inhibitor of an EMT-TF; and (b) an inhibitor of an EMT-cooperating TF.

78. The method of claim 77, wherein the subject in need of treatment of a tumor that expresses high levels of the EMT-TF and the EMT-cooperating TF.

79. The method of claim 77, wherein the EMT-cooperating protein is Sox9 or Sox10.

80. The method of claim 77, wherein the tumor is a breast cancer, and the EMT-cooperating protein is Sox9 or Sox10.

81. The method of claim 77, wherein the tumor is a breast cancer, the EMT-TF is Slug or Snail, and the EMT-cooperating protein is Sox9 or Sox10.

82. A method of classifying a cell, sample, or tumor, the method comprising: (a) assessing expression of at least two genes in the cell, sample, or tumor, wherein the first gene encodes or is regulated by an EMT-TF and the second gene encodes or is regulated by an EMT-cooperating TF, wherein increased expression of the first and second genes is correlated with a phenotypic characteristic, thereby classifying the cell, sample, or tumor with respect to the phenotypic characteristic.

83. The method of claim 82, wherein the EMT-TF is Slug or Snail.

84. The method of claim 82, wherein the EMT-cooperating TF is a Sox protein.

85. The method of claim 82, wherein the EMT-cooperating TF is Sox 9 or Sox 10.

86. The method of claim 82, wherein the cell is not a tumor cell, and wherein an increased level of expression of the first and second genes indicates that the cell is a stem cell.

87. The method of claim 82, wherein the cell is a tumor cell, and wherein an increased level of expression of the first and second genes indicates that the tumor cell has increased tumor-initiating or metastatic ability.

88. The method of claim 82, wherein the tumor cell is a breast tumor cell, the EMT-cooperating TF is Slug or Snail, and the EMT-cooperating protein is Sox 9 or Sox10.

89. The method of claim 82, wherein the method comprises classifying a tumor, and wherein increased expression of both the first and the second genes in one or more samples obtained from the tumor indicates an increased likelihood of poor outcome.

90. A method of identifying a stem cell comprising steps of: (a) providing a sample comprising at least one cell; (b) assessing expression of a first gene that encodes an EMT-TF and a second gene that encodes or is regulated by an EMT-cooperating TF in at least one cell of the sample; and (c) identifying a cell that has increased expression of the first and second genes, thereby identifying a stem cell.

91. The method of claim 90, wherein the sample comprises normal cells, and the method comprises identifying a normal cell that has increased expression of the first and second genes, thereby identifying a normal stem cell.

92. The method of claim 90, wherein the sample comprises tumor cells, and the method comprises identifying a tumor cell that has increased expression of the first and second genes, thereby identifying a cancer stem cell.

93. The method of claim 90, wherein the sample comprises multiple cells, and the method further comprises separating at least one cell that has increased expression of the first and second genes from at least one cell that does not have increased expression of both of the genes.

94. A method of identifying an EMT-cooperating agent, the method comprising: (a) contacting a plurality of differentiated epithelial cells with an EMT-inducing agent and a test agent; (b) maintaining the cells for a suitable time period; (c) assessing the cells for one or more SC properties; and (d) identifying the test agent as an EMT-cooperating agent if the cells exhibit an increase in one or more SC properties as compared with control cells.

95. The method of claim 94, wherein contacting the differentiated epithelial cells with an EMT-inducing agent comprises causing the cells to express or overexpress an EMT-TF.

96. The method of claim 94, wherein contacting the differentiated epithelial cells with an EMT-inducing agent comprises causing the differentiated epithelial cells to express or contain an EMT-TF that is naturally absent or weakly expressed by said cells.

97. The method of claim 94, wherein the test agent comprises a protein and contacting the differentiated epithelial cells with a test agent comprises causing the cells to express the protein.

98. The method of claim 94, wherein the test agent comprises a TF and contacting the differentiated epithelial cells with a test agent comprises causing the cells to express the TF.

99. A method of culturing an epithelial cell, the method comprising: (a) providing an epithelial cell; (b) culturing the epithelial cell in culture medium comprising a ROCK inhibitor.

100. The method of claim 99, further comprising generating a stem cell from the epithelial cell.

101. The method of claim 99, further comprising inducing EMT in the epithelial cell.

102. The method of claim 99, further comprising expressing an EMT-cooperating TF in the cell.

103. The method of claim 99, further comprising inducing EMT and expressing an EMT-cooperating TF in the epithelial cell

104. A method of culturing a stem cell, the method comprising: (a) providing a stem cell; and (b) culturing the stem cell in culture medium comprising a ROCK inhibitor.

105. The method of claim 104, wherein the culture medium comprises about 5% Matrigel or an equivalent thereof.

106. The method of claim 104, wherein the stem cell is an epithelial stem cell.

107. The method of claim 104, wherein the stem cell is a mammary epithelial stem cell.

108. The method of claim 104, wherein the stem cell is cultured for a sufficient period of time to generate an organoid.

109. A composition or kit comprising a ROCK inhibitor and an isolated EMT-inducing agent.

110. The composition or kit of claim 109, further comprising an isolated EMT-cooperating agent.

111. The composition or kit of claim 109, wherein the isolated EMT-inducing agent comprises a nucleic acid that encodes a polypeptide comprising an EMT-TF.

112. The composition or kit of claim 109, further comprising an isolated EMT-cooperating agent comprising a nucleic acid that encodes a polypeptide comprising an EMT-cooperating TF.

113. A composition comprising a ROCK inhibitor and about 5% Matrigel or an equivalent thereof.

114. The composition of claim 113, further comprising a population of epithelial cells.

115. A method of obtaining an organoid from a stem cell, the method comprising culturing a stem cell in a composition comprising about 5% Matrigel or an equivalent thereof.

116. The method of claim 115, further comprising isolating the organoid from the composition and, optionally, analyzing the organoid.

117. The method of claim 115, further comprising isolating the organoid from the composition and analyzing the organoid, wherein analyzing the organoid comprises implanting the organoid into a subject and assessing the development of the organoid in the subject.

118. The method of claim 115, further comprising isolating the organoid from the composition and implanting the organoid into a subject.

119. The method of claim 115, wherein the stem cell is an epithelial stem cell

120. The method of claim 115, wherein the stem cell is a mammary epithelial stem cell.

121. A composition comprising at least one organoid in a composition comprising about 5% Matrigel or an equivalent thereof.

122. The composition of claim 121, wherein the organoid is a mammary organoid.

123. The composition of claim 121, wherein the composition comprises a ROCK inhibitor.

124. A method of generating a cancer stem cell (CSC) comprising: (a) providing a tumor cell; (b) generating astern cell from the tumor cell according to the method of any of claims 1-24, 32-36, or 38-42, thereby generating a cancer stem cell.

125. A method of identifying an agent that inhibits survival or proliferation of cancer stem cells (CSCs), the method comprising: (a) providing a population of CSCs generated according to the method of claim 124; (b) contacting the cells with a candidate agent; and (c) assessing survival or proliferation of the cells, wherein a decrease in survival or proliferation of the cells as compared with a control indicates that the agent inhibits survival or proliferation of CSCs.

126. The method of any of claims 1-41, 52, 70-76, 82-108, or 115-120, or 125, wherein the cell(s) are human cells.

127. A method of treating a subject comprising: (a) obtaining stem cells or differentiated epithelial cells according to the method of any of claims, and (b) introducing at least some of the cells into a subject in need thereof.

128. The composition or cell of any of claims 50, 51, 53-69, or 121-123, wherein the cell(s) are human.

129. The method of any of the foregoing claims that pertain at least in part to a subject, wherein the subject is human.

130. The method of any of the foregoing claims that pertain at least in part to an EMT-TF or EMT-cooperating protein, wherein the EMT-TF or EMT-cooperating protein is human.
